(19) United States
(12) Patent Application Publication
(10) Pub. No.: US 2026/0327300 A1
(43) Pub. Date: Oct. 22, 2026

(54) INTERFERON LAMBDA (IFNL) INHIBITION IN INTESTINAL EPITHELIAL CELLS FOR TREATING INFLAMMATION

(71) Applicant: The children's Medical Center Corporation, Boston, MA (US)

(72) Inventors: Ivan Zanoni, Brookline, MA (US); Scott Snapper, Wellesley, MA (US); Achille Broggi, Marseille (FR)

(73) Assignee: The children's Medical Center Corporation, Boston, MA (US)

(21) Appl. No.: 19/114,028

(22) PCT Filed: Sep. 22, 2023
(86) PCT No.: PCT/US2023/074874

§ 371 Date: Mar. 21, 2025

Related U.S. Application Data
(60) Provisional application No. 63/409,676, filed on Sep. 23, 2022.

(51) Int. Cl. C07K 16/24; A61K 31/4178; A61K 38/17; A61K 38/46; A61P 1/00; C07K 16/28; C07K 16/40

(52) U.S. Cl. CPC C07K 16/244; A61K 31/4178; A61K 38/1761; A61K 38/465; A61P 1/00; C07K 16/2866; C07K 16/40; C07K 2317/76

(57) ABSTRACT

Provided herein are methods for treating inflammatory diseases, such as inflammatory bowel disease (IBD), by administering to a subject an agent to inhibit interferon lambda (IFNL) signaling in intestinal epithelial cells. Such methods may be used to reduce inflammation, reduce cell death, increase epithelial cell proliferation, and/or increase tissue repair in intestinal epithelia of patients having an inflammatory intestinal disease.

### RELATED APPLICATION

This application claims the benefit under 35 U.S.C. § 119(e) of U.S. Provisional Application No. 63/409,676, filed Sep. 23, 2022, entitled “INTERFERON LAMBDA (IFNL) INHIBITION IN INTESTINAL EPITHELIAL CELLS FOR TREATING INFLAMMATION, “the entire contents of which are incorporated herein by reference.

### GOVERNMENT SUPPORT

This invention was made with government support under grant number DK115217 awarded by the National Institutes of Health (NIH). The government has certain rights in the invention.

### REFERENCE TO AN ELECTRONIC SEQUENCE LISTING

The contents of the electronic sequence listing (C123370260WO00-SEQ-VLJ.xml; Size: 4,650 bytes; and Date of Creation: Aug. 30, 2023) is herein incorporated by reference in its entirety.

### BACKGROUND

Interferons (IFNs) are a class of cytokines that are released by certain types of immune cells during an immune response, particularly in response to infection by a pathogenic bacterium or virus. Secreted interferons act by binding to specific interferon receptors on the surface of cells, which in turn triggers downstream signaling cascades that mediate immune responses by modulating the expression level of various immunoactive proteins inside cells. Interferons encoded by humans includes type I interferons (e.g., interferon alpha (IFNA), interferon beta (IFNB), interferon kappa (IFNK), interferon omega (IFNW)), type II interferons (e.g., interferon gamma (IFNG)), and type III interferons (e.g., interferon lambda (IFNL)). Each type of interferon has been shown to stimulate inflammation in certain contexts, therefore inhibition of interferon signaling could potentially be used to treat inflammation caused by overactive immunity. To this end, several inhibitors, primarily monoclonal antibodies, have been identified that specifically bind and inhibit IFNA or IFNA receptor (IFNAR). However, to date no therapeutics have been developed that modulate type III interferon activity, specifically that which is mediated by IFNL and IFNL receptor (IFNLR).

### SUMMARY

The present disclosure is based on the discovery that interference with interferon lambda (IFNL) signaling in intestinal epithelia reduces cell death and enhances tissue repair occurring in said epithelia in the context of certain inflammatory diseases. Accordingly, some aspects of the present disclosure relate to a method for treating inflammatory bowel disease (IBD) in a subject, the method comprising administering to the subject an effective amount of an agent that is sufficient to inhibit interferon lambda (IFNL) signaling in intestinal epithelial cells of the subject.

In some embodiments, the agent comprises an antibody, a small molecule, a nucleic acid, or a gene editing agent.

In some embodiments, the antibody binds to IFNL or interferon lambda receptor (IFNLR). In some embodiments, the antibody specifically binds to IFNL or IFNLR. In some embodiments, the antibody binds to IFNL or IFNLR on the cell surface of intestinal epithelial cells of the subject. In some embodiments, the antibody inhibits the activity of IFNL or IFNLR in intestinal epithelial cells of the subject. In some embodiments, the antibody inhibits the activity of IFNL or IFNLR in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the small molecule binds to interferon lambda receptor (IFNLR). In some embodiments, the small molecule binds to IFNLR on the cell surface of intestinal epithelial cells of the subject. In some embodiments, the small molecule inhibits the activity of IFNLR in intestinal epithelial cells of the subject. In some embodiments, the small molecule inhibits the activity of IFNLR in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the small molecule binds to a Janus kinase (JAK) protein that is bound to interferon lambda receptor (IFNLR) on the cell surface of intestinal epithelial cells of the subject. In some embodiments, the JAK protein is Janus kinase 2 (JAK2). In some embodiments, the small molecule does not bind to a JAK protein that is bound to interferon alpha receptor (IFNAR) on the cell surface of intestinal epithelial cells of the subject. In some embodiments, the small molecule inhibits the activity of the JAK protein in intestinal epithelial cells of the subject. In some embodiments, the small molecule inhibits the activity of the JAK protein in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the small molecule binds to a Viperin (RSAD2), protein. In some embodiments, the small molecule inhibits the activity of the Viperin (RSAD2) protein in intestinal epithelial cells of the subject. In some embodiments, the small molecule inhibits the activity of the Viperin (RSAD2) protein in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the small molecule binds to a factor of PANoptosis signaling in intestinal epithelial cells of the subject. In some embodiments, the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein. In some embodiments, the caspase protein is caspase-8, caspase-3, caspase-7, or caspase-1. In some embodiments, the gasdermin protein is Gasdermin C or Gasdermin D. In some embodiments, the factor of PANoptosis signaling is RIPK1 and the small molecule is necrostatin. In some embodiments, the small molecule inhibits the activity of the factor of PANoptosis signaling in intestinal epithelial cells of the subject. In some embodiments, the small molecule is selected from Z-VAD-FMK or Z-IETD-FMK. In some embodiments, the small molecule blocks the z-nucleic acid binding site of Z-DNA-binding protein 1 (ZBP1). In some embodiments, the small molecule inhibits the activity of the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the nucleic acid is a small interfering RNA (siRNA) or a short hairpin RNA (shRNA). In some embodiments, the nucleic acid inhibits the expression of interferon lambda receptor (IFNLR), a Janus kinase (JAK) protein, Viperin (RSAD2), or a factor of PANoptosis signaling in intestinal epithelial cells of the subject. In some embodiments, the JAK protein is Janus kinase 2 (JAK2). In some embodiments, the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein. In some embodiments, the caspase protein is caspase-8, caspase-3, caspase-7, or caspase 1. In some embodiments, the gasdermin protein is Gasdermin C or Gasdermin D. In some embodiments, the nucleic acid blocks the z-nucleic acid binding site of Z-DNA-binding protein 1 (ZBP1). In some embodiments, the nucleic acid inhibits the expression of IFNLR, the JAK protein, or the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the gene editing agent comprises a zinc finger nuclease (ZFN), a transcription activator-like effector nuclease (TALEN), or a clustered regularly interspaced short palindromic repeat (CRISPR)-Cas-associated nuclease (CRISPR/CAS). In some embodiments, the gene editing agent comprises CRISPR/CAS and further comprises a short guide RNA (sgRNA) that is complementary to a gene encoding interferon lambda receptor (IFNLR), a Janus kinase (JAK) protein, Viperin (RSAD2), or a factor of PANoptosis signaling in intestinal epithelial cells of the subject. In some embodiments, the gene editing agent binds to and modifies a gene encoding interferon lambda receptor (IFNLR), a Janus kinase (JAK) protein, or a factor of PANoptosis signaling in intestinal epithelial cells of the subject. In some embodiments, the JAK protein is Janus kinase 2 (JAK2). In some embodiments, the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein. In some embodiments, the caspase protein is caspase-8, caspase-3, caspase-7, or caspase-1. In some embodiments, the gasdermin protein is Gasdermin C or Gasdermin D. In some embodiments, the gene editing agent reduces the expression of IFNLR, the JAK protein, or the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the agent further comprises a delivery agent. In some embodiments, the delivery agent is a peptide, an antibody, a liposome, or a viral particle. In some embodiments, the delivery agent binds specifically to the cell surface of intestinal epithelial cells of the subject. In some embodiments, the agent and the delivery agent are covalently linked. In some embodiments, the agent and the delivery agent are linked by a cleavable linker. In some embodiments, the cleavable linker is a protease-sensitive linker, a pH-sensitive linker, or a glutathione-sensitive linker. In some embodiments, the agent and the delivery agent are linked by a non-cleavable linker. In some embodiments, the delivery agent enhances delivery of the agent to intestinal epithelial cells of the subject, as compared to delivery of the agent to intestinal epithelial cells of the subject in the absence of the delivery agent. In some embodiments, the delivery agent enhances internalization of the agent by intestinal epithelial cells of the subject, as compared to internalization of the agent by intestinal epithelial cells of the subject in the absence of the delivery agent.

In some embodiments, the method results in a reduction of inflammation in intestinal epithelial cells of the subject. In some embodiments, the method results in a reduction of cell death in intestinal epithelial cells of the subject. In some embodiments, the cell death is cell death as a result of apoptosis, pyroptosis, and/or necroptosis. In some embodiments, the method results in increased proliferation of intestinal epithelial cells of the subject. In some embodiments, the method results in an enhancement of tissue repair in intestinal epithelial cells of the subject.

In some embodiments, the IBD is Crohn's disease (CD). In some embodiments, the IBD is ulcerative colitis (UC).

In some embodiments, the subject is a mammal. In some embodiments, the subject is a human patient.

In some embodiments, the administration is oral or via injection. In some embodiments, the administration is systemic. In some embodiments, the administration is local. In some embodiments, the administration is rectal. In some embodiments, the agent is administered to intestinal epithelial cells of the subject. In some embodiments, the agent is administered to the subject more than once.

### BRIEF DESCRIPTION OF DRAWINGS

The accompanying drawings are not intended to be drawn to scale. In the drawings, each identical or nearly identical component that is illustrated in various FIGS. is represented by a like numeral. For purposes of clarity, not every component may be labeled in every drawing.

In the drawings:

FIGS. 1A-1D show reconstitution of in vivo interferon lambda (IFNL) signaling in an in vitro organoid model. FIG. 1A shows a schematic for an in vitro assay utilizing murine or human small intestinal organoids maintained under an air-liquid interface (ALI). Submerging the cultivated organoid monolayer mimics the effects of tissue damage due to inflammation. Recombinant IFNL may be added prior to or following the submersion step to activate IFNL signaling in the monolayer. FIG. 1B shows immunohistochemical staining of control intestinal organoid monolayers and monolayers treated with recombinant IFNL. Monolayers were treated with IFNL, as appropriate, prior to submersion. DAPI staining (middle panel, left) indicates the presence of nuclei, ZombieRed staining (middle panel, right) indicates the presence of dead cells, NucView staining (lower panel, left) indicates the caspase-3 activation, and Ki67 staining (lower panel, right) indicates proliferative cells. FIG. 1C shows quantification of indicated immunohistochemical readouts shown in FIG. 1B, at 24 hours and 72 hours following treatment with recombinant IFNL to activate IFNL signaling. FIG. 1D shows quantification of cell survival over time in monolayers treated with the caspase-3 inhibitor Z-IETD-FMK (ZIETD). Z-IETD-FMK was added to organoid cultures 30 minutes prior to treatment with recombinant IFNL. Cell survival was assessed by propidium iodide staining. Right panel shows area under the curve of quantified cell survival over time. The number of surviving cells in the presence of Z-IETD-FMK did not significantly differ between untreated cultures and cultures treated with recombinant IFNL. *: p<0.05; n.s.: not significant.

FIGS. 2A-2I show how IFN-λ inhibits tissue recovery after DSS-colitis. FIGS. 2A-2C show how WT mice were treated with 2.5% DSS for 7 days (the dotted line indicates the end of DSS administration). Upon DSS withdrawal on Day 7, mice were injected intraperitoneally (i.p.) with 50 μg kg⁻¹ day⁻¹ of rIFN-λ for five consecutive days. Weight (FIG. 2A), colon length (FIG. 2B), histological score and representative histology images (FIG. 2C) are depicted. FIGS. 2D-2E show how WT mice were treated with DSS for 7 days as in (FIGS. 2A-2C). Upon DSS withdrawal mice were injected i.p. with either 50 μg kg⁻¹ day⁻¹ of rIFN-λ or 12.5 mg kg⁻¹ day-1 of anti-IFN-λ2,3 antibody. Weight (FIG. 2D), and colon length (FIG. 2E) are depicted. FIGS. 2F-2G show how mice were treated with DSS as in (FIGS. 2A-2C). Upon DSS withdrawal mice were injected i.p. with 50 mg kg⁻¹ day⁻¹ of rIFN-α or 12.5 mg kg⁻¹ day⁻¹ of anti-IFNAR1 antibody. Weight (FIG. 2F), and colon length (FIG. 2G) are depicted. FIGS. 2H-2I show how WT (FIG. 2H) or Vil^{CRE}Ifnlr1^{fl/fl} mice (FIG. 2H) were treated with 2.5% DSS for seven days (dotted line). Upon DSS withdrawal mice were injected i.p. with 50 μg kg⁻¹ day⁻¹ of rIFN-λ. Weight (FIG. 2H), and colon length (FIG. 2I) are depicted. FIGS. 2A, 2D, 2F and 2H show the mean and SEM of 5 mice per group. Two-way ANOVA with Tukey correction for multiple comparisons was utilized. FIGS. 2B, 2C, 2E, 2G depict box plots. In FIG. 2I WT or Vil^{CRE}Ifnlr1^{fl/fl} mice were treated as in (2B, 2E) and colon lengths were assessed on day 10 after start of DSS treatment. Each dot represents a mouse. Median, range and interquartile range are depicted. FIGS. 2B-2C show the statistics of an unpaired t test. FIGS. 2E and 2G show a one-way ANOVA with Dunnett correction for multiple comparisons. FIG. 2I shows a two-way ANOVA with Šidak correction for multiple comparisons. Data representative of 3 independent experiments. ns=not significant (p>0.05); *p<0.05; **p<0.01; ***p<0.001; ****p<0.0001.

FIGS. 3A-3E show how IFN-λ impairs epithelial regeneration after radiation damage. FIG. 3A depicts WT mice and Ifnlr1^{-/-} received 11 Gy ionizing radiation, with lead shielding of the upper body. WT mice were either administered 50 mg kg⁻¹ day⁻¹ of rIFN-λ (WT+rIFN-λ), or the same volume of saline vehicle (WT+Veh). Tissue repair in the small intestine was evaluated 96 hours after irradiation by counting the number of intact crypts per histological section (Crypts/section). Representative histological images and their corresponding quantification are shown. FIG. 3B shows WT, Ifnlr1^{-/-}, or Ifnar1^{-/-} mice were irradiated as in FIG. 3A. Quantification of intact crypts per histological section is depicted. FIG. 3C Vil^{CRE} Ifnlr1^{fl/fl} mice or WT mice were irradiated as in FIG. 3A and treated with either 50 mg kg⁻¹ day⁻¹ of rIFN-λ (rIFN-λ), or saline vehicle (Veh). Quantification of intact crypts per histological section is depicted. FIGS. 3D and 3E depict Vil^{CRE} Ifnlr1^{fl/fl} mice or WT mice were irradiated with 14 Gy of ionizing radiation and treated with either 50 mg kg⁻¹ day⁻¹ of rIFN-λ (rIFN-λ), or saline vehicle (Veh) and followed over time. Weight (FIG. 3D) and survival (FIG. 3E) are depicted. Statistical comparison between “WT+Veh” and “Vil^{CRE}Ifnlr1^{fl/fl}+Veh” are depicted as (*), comparison between “WT+Veh” and “WT+rIFN-λ” is depicted as (§). FIGS. 3A-3C depict box plots. Each dot represents a mouse. Median, range and interquartile range are depicted. FIG. 3D depicts the Mean and SEM. FIGS. 3A-3B depict the statistics of a One-way ANOVA with Dunnett correction for multiple comparisons. FIG. 3C shows the two-way ANOVA with Šidak correction for multiple comparison. Vil^{CRE} Ifnlr1^{fl/fl} mice or WT mice were irradiated and treated with either 50 μg kg⁻¹ day⁻¹ of rIFN-λ (rIFN-λ), or saline vehicle (Veh). Left panel: Quantification of intact crypts per histological section; right panel: Representative histological images. FIG. 3D shows the two-way ANOVA with Tukey correction for multiple comparisons. ns=not significant (p>0.05); *p<0.05; **p<0.01; ***p<0.001; ****p<0.0001.

FIGS. 4A-4F show how IFN-λ signaling induces an antiproliferative program in small intestine epithelia. FIGS. 4A-4D, and FIG. 4F show that Vil^{CRE} Ifnlr1^{fl/fl} mice or WT mice received 11 Gy ionizing radiation, with lead shielding of the upper extremities. Targeted transcriptomics was performed on small intestinal crypts isolated 96 hours after irradiation. FIG. 4A shows dot plots of Gene Ontology (GO) enrichment analysis. GO terms enriched in crypts from WT mice or Vil^{CRE} Ifnlr1^{fl/fl} mice are shown. Gene ratio (x axis), adjusted p-value and gene count (dot size) are depicted. FIGS. 4B-4D show GSEA enrichment plots of the HALLMARK_IFN_ALPHA_RESPONSE (FIG. 4B), REGENERATIVE SIGNATURE (as previously described (Yui, Azzolin et al. 2018)) (FIG. 4C), and the GO biological process Cell Population proliferation (“GOBP_CELL_POP_PROLIF) (FIG. 4D) are depicted. padj: adjusted p-value, NES: Normalized enrichment score, SIZE: size. FIG. 4E shows that Vil^{CRE} Ifnlr1^{fl/fl} mice or WT mice were irradiated as in FIGS. 4A-4D and treated with either 50 mg kg⁻¹ day⁻¹ of rIFN-λ (rIFN-λ), or saline vehicle (Veh). After 96 hours mice were pulsed with EdU for 2 hours. Number of EdU⁺ cells per crypt quantified by immunohistochemistry (IHC), and representative IHC sections are depicted. FIG. 4F shows the targeted transcriptomics data from WT or Vil^{CRE} Ifnlr1^{fl/fl} small intestinal crypts were deconvoluted based on publicly available single-cell RNA-seq (scRNA-seq) datasets (Haber, Biton et al. 2017) using CIBERSORTx (Newman, Steen et al. 2019) to extrapolate the relative cellular composition of samples. Paneth: Paneth cells; Stem: Intestinal stem cells; Enterocytes: small intestine enterocytes; TA: Transit amplifying cells; Goblet: Goblet cells; Tuft: tuft cells; EEC: Enteroendocrine cells; EP: Enterocyte progenitors. FIG. 4E depicts box plots. Each dot represents a mouse. Median, range and interquartile range are depicted. FIG. 4F shows the Mean and SEM of 4 samples (WT) and 3 samples (Vil^{CRE} Ifnlr1^{fl/fl}) are depicted. FIG. 4E used a two-way ANOVA with Turkey correction for multiple comparisons. FIG. 4F show the statistics of the two-way ANOVA with Šidak correction for multiple comparisons. ns=not significant (p>0.05); *p<0.05; **p<0.01; ***p<0.001; ****p<0.0001.

FIGS. 5A-5D show how IFN-λ controls the expression of ZBP1 and the activation of gasdermin C. FIG. 5A shows that Vil^{CRE} Ifnlr1^{fl/fl} mice or WT mice were irradiated as in FIG. 3A. Targeted transcriptomics was performed on freshly isolated small intestinal crypts. Volcano plot depicting differentially expressed genes (DEGs) between Vil^{CRE} Ifnlr1^{fl/fl} and WT small intestinal crypts. DEGs (PC 0.005) with a fold change >2 (or <−2) are indicated; DEGs with a fold change <2 (or >−2) are indicated. Nonsignificant DEGs (P>0.005) and genes not differentially expressed are indicated in green and gray, respectively. Positive values represent genes overexpressed in Vil^{CRE}Ifnlr1^{fl/fl}, negative values represent genes overexpressed in WT. FIG. 5B shows that RNA sequencing was performed on colon biopsies from control patients, IBD patients with inactive disease, and IBD patients with active disease, (see Materials and Methods). Square symbols represent controls, round symbols represent ulcerative colitis (UC) patients, triangles represent Crohn's disease patients (CD). Box plots with median, range and interquartile range are depicted. Each symbol represents one patient. Expression of GSDMC, ZBP1, CASP8 expressed as normalized log 2 count is depicted from left to right top to bottom. Mean expression of genes belonging to the GSEA HALLMARK_IFN_ALPHA_RESPONSE gene set (IFN Response score), is depicted. FIG. 5C shows a dot plot depicting the correlation between GSDMC expression and the IFN Response score performed on the same samples as FIG. 5B. Each point represents a patient, solid lines represent linear regression, shaded area depicts the confidence interval. Spearman correlation coefficient (rho) and the relative p-value (pval) are indicated for each graph. FIG. 5D shows small intestinal crypts were isolated from Vil^{CRE} Ifnlr1^{fl/fl} mice or WT mice irradiated as in FIG. 3A (Irrad+) or not (Irrad−). Immunoblot analysis of the indicated proteins was performed. GSDMC-2/-3 FL p50: full length 50 kDa GSDMC-2/3; GSDMC-2/-3 CL p30: N-terminal 30 kDa cleaved protein; GSDMD FL p50: full length GSDMD 50 kDa; GSDMD CL p30: N-terminal 30 kDa cleaved protein; CASP-8 FL: full length CASP-8; CASP-8 p18: 18 kDa CASP-8 cleavage fragment; CASP-3 FL: full length CASP-3; CASP-3 p17: 17 kDa CASP-3 cleavage fragment; CASP-3 p12: 12 kDa CASP-3 cleavage fragment. Each lane represents one mouse. Representative data of 3 independent experiments is depicted. FIG. 5B shows the statistics of the Kruskal Wallis test with Dunn correction for multiple comparisons was performed. ns=not significant (p>0.05); *p<0.05; **p<0.01; ***p<0.001; ****p<0.0001.

FIGS. 6A-6I show how IFN-λ inhibits epithelial proliferation and survival in intestinal organoids in vitro. FIG. 6A shows that mouse small intestinal organoids were seeded from freshly isolated crypts and allowed to grow for 48 hours. Organoids were then treated with 200 ng/ml of rIFN-λ in the presence of 1 ug/ml propidium iodide (PI) and imaged every 12 hours over 72 hours. Percentage of live organoids was calculated as percentage of PI⁻ organoids over the total number of live organoids in each well. Representative image of 3 independent experiments is depicted. FIG. 6B shows small intestinal organoids derived from WT or Stat1^{-/-} mice were seeded from freshly isolated crypts and allowed to grow for 48 hours. Organoids were treated with 200 ng/ml of rIFN-λ in the presence of 1 μg/ml propidium iodide (PI) for 72 hours. Organoids were treated as in FIG. 6A. Percentage of live organoids was calculated as percentage of PI⁻ organoids over the total number of live organoids in each. FIG. 6C shows that small intestinal organoids were seeded and treated as in FIG. 6A for 72 hours. Organoids were pulsed with EdU for 6 hours. Organoids were stained for EdU incorporation, and DAPI. Mean fluorescence of EdU staining (left), relative organoid growth (middle) and representative images (right), are depicted. The relative growth of organoids is measured as the % of their area over untreated control organoids. FIG. 6D shows the small intestinal organoids derived from WT or Zbp1^{-/-} mice were seeded and treated with 200 ng/ml of rIFN-λ and imaged at 48 hours. The relative growth of organoids measured as in FIG. 6C is depicted. FIG. 6E shows that the small intestinal organoids were treated as in FIG. 6A for 24, 48 and 72 hours. Immunoblot analysis of the indicated proteins was performed. GSDMC-2/-3 FL p50: full length 50 kDa GSDMC-2/-3; GSDMC-2/-3 CL p30: N-terminal 30 kDa cleaved protein; c-CASP-8 p43: 43 kDa CASP-8 cleavage fragment; c-CASP-8 p18: 18 kDa CASP-8 cleavage fragment. Representative blot of three independent experiments. FIG. 6F shows that the small intestinal organoids were seeded as in FIG. 6A and either treated with 200 ng/ml of rIFN-λ alone (rIFN-λ) or with rIFN-λ in the presence of the pan caspase inhibitor Z-VAD-FMK (40 uM). Organoids were then followed for 72 hours and % of live organoids was evaluated as in FIG. 6A. Statistical comparison between “rIFN-λ” and “Veh” are depicted (*), comparison between “rIFN-λ+Z-VAD-FMK” and “rIFN-λ” is depicted (§). FIG. 6G shows that the small intestinal organoids were either left untreated (WT) or infected with a lentivirus expressing GFP and either a Gasdermin-2/Gasdermin-3 targeting (Gsdmc2, 3^{KD}) small harpin (sh)RNA, or a scrambled control shRNA (Scramble). Organoids were grown for 5 days and then treated with 200 ng/ml of rIFN-λ or vehicle control for 48 h. Survival of WT controls or lentiviral-infected GFP⁺ cells was assessed by cytofluorimetry by staining with Zombie dye and calcein. % of dead cells represent cells positive for Zombie dye staining and negative for calcein. FIG. 6H shows the experimental scheme for the establishment of 2D Air Liquid Interface (ALI) organoid cultures and modeling of damage and repair responses. Organoids were seeded in transwells and grown to confluence. The apical side was then exposed to air up to 14 days, which favored differentiation of a homeostatic monolayer. Organoids were then submerged for 7 days to induce damage responses. After 7 days they were re-exposed to air to stimulate repair responses. Concomitantly with re-exposure to air, organoids were treated with 200 ng/ml of rIFN-λ for 3 days. FIG. 6I shows the organoids treated as in FIG. 6H were pulsed with EdU for 2 hours to mark proliferating cells. Quantification of the percentage of EdU⁺ cells per field of view (left) and representative images (right) are depicted. Edu, and DNA stain DAPI are depicted. Representative image of 3 independent experiments. FIG. 6A and FIG. 6F show the Mean and SEM of 3 (FIG. 6A) and 5 (FIG. 6F) biological replicates per group are depicted. FIGS. 6B-6C depict box plots. Each dot represents a biological replicate. Median, range and interquartile range are depicted. FIGS. 6D, 6G, and 6I depict scatter plots. Each dot represents a biological replicate. FIGS. 6A and 6F depict the statistics of the two-way ANOVA with Tukey correction for multiple comparisons. FIGS. 6B, 6C, 6D, 6G, and 6I depict the two-way ANOVA with Šidak correction for multiple comparison. FIG. 6C shows the unpaired t test. ns=not significant (p>0.05); *or § p<0.05; **or §§ p<0.01; ***or § § § p<0.001; ****or § § § § p<0.0001.

FIGS. 7A-7C show how IFN-λ inhibits tissue recovery after DSS-colitis. FIG. 7A shows that WT mice were treated with 2.5% DSS for 7 days. Upon DSS withdrawal mice were injected i.p. with either 50 μg kg-1 day-1 of rIFN-λ or 12.5 mg kg⁻¹ day⁻¹ of anti-IFN-λ2,3 antibody for five days. Rsad2 relative mRNA expression in colonocytes on Day 14 is depicted. FIG. 7B shows that mice were treated with DSS as in FIG. 7A. Upon DSS withdrawal mice were injected i.p. with either 50 mg kg⁻¹ day⁻¹ of rIFN-λ or 12.5 mg kg⁻¹ day⁻¹ of anti-IFNAR1 antibody. Rsad2 relative mRNA expression in colonocytes on Day 14 is depicted. FIG. 7C shows WT (left panel) or Mrp8^{CRE}Ifnlr1^{fl/fl} mice (right panel) were treated with 2.5% DSS for seven days. Upon DSS withdrawal mice were injected i.p. with 50 μg kg⁻¹ day⁻¹ of rIFN-λ. Weight is depicted. FIGS. 7A-7B depict box plots. Each dot represents a mouse. Median, range and interquartile range are shown. FIG. 7C depicts the Mean and SEM of 5 mice per group are depicted. FIGS. 7A-7B show the statistics of a one-way ANOVA with Dunnett correction for multiple comparisons. FIG. 7C shows the two-way ANOVA with Tukey correction for multiple comparisons. ns=not significant (p>0.05); *p<0.05; **p<0.01; ***p<0.001; ****p<0.0001.

FIGS. 8A-8D show how IFN-λ impairs epithelial regeneration after radiation damage. FIG. 8A shows WT, Ifnlr1^{-/-}, or Ifnar1^{-/-} mice received 11 Gy ionizing radiation, with lead shielding of the upper body. Rsad2 relative mRNA expression in small intestinal crypt cells was evaluated 96 hours after irradiation. FIG. 8B shows Vil^{CRE} Ifnlr1^{fl/fl} mice or WT mice were irradiated and treated with either 50 mg kg⁻¹ day⁻¹ of rIFN-λ (rIFN-λ), or saline vehicle (Veh). Tissue repair in the small intestine was evaluated 96 hours after irradiation. Representative histological images of the small intestine are depicted. FIG. 8C shows WT mice and Mrp8^{CRE}Ifnlr1^{fl/fl} were irradiated as in FIG. 8A. Tissue repair in the small intestine was evaluated 96 hours after irradiation by counting the number of intact crypts per histological section (Crypts/section). FIG. 8D shows the number of small intestinal intact crypts per histological section (Crypts/section) was evaluated in WT and Ifnlr1^{-/-} (left panel) and WT and Vil^{CRE}Ifnlr1^{fl/fl} (right panel) mice at homeostasis. FIG. 8A depicts box plots. Each dot represents a mouse. Median, range and interquartile range are depicted. FIGS. 8C-8D depict scatter plots. Each dot represents a mouse. Mean with SEM are depicted. FIG. 8A shows the statistics of the one-way ANOVA with Dunnett correction for multiple comparisons. FIG. 8B shows the unpaired t test. ns=not significant (p>0.05); *p<0.05; **p<0.01; ***p<0.001; ****p<0.0001.

FIGS. 9A-9E show how IFN-λ signaling induces an antiproliferative program in small intestine epithelia. FIGS. 9A-9D show that Vil^{CRE} Ifnlr1^{fl/fl} mice or WT mice received 11 Gy ionizing radiation, with lead shielding of the upper body. FIGS. 9A-9B show targeted transcriptomics was performed on small intestinal crypts isolated 96 hours after irradiation. FIG. 9A shows a heatmap depicting expression of genes in the leading edge of the enriched HALLMARK_IFN_ALPHA_RESPONSE gene set. The color is proportional to the Z Score. FIG. 9B shows a heatmap depicting expression of genes in the leading edge of the REGENERATION SIGNATURE gene set. The grayscale is proportional to the Z Score. FIG. 9C shows a heatmap depicting expression of genes in the leading edge of the enriched GOBP_CELL_POPULATION_PROLIFERATION gene set. The grayscale is proportional to the Z Score. FIG. 9D shows Lgr5, Lyz1, Muc2, Chga relative mRNA expression in small intestinal crypt cells was evaluated 96 hours after irradiation. FIG. 9E shows Lgr5, Lyz1, Muc2, Chga relative mRNA expression in small intestinal crypt cells isolated from Vil^{CRE} Ifnlr1^{fl/fl} mice or WT mice at irradiation (FIG. 9D) or at homeostasis (FIG. 9E) was evaluated. FIGS. 9D-9E depict box plots. Each dot represents a mouse. Median, range and interquartile range are depicted. FIGS. 9D-9E show the unpaired t test. ns=not significant (p>0.05); *p<0.05; **p<0.01.

FIGS. 10A-10B show how IFN-λ controls the expression of ZBP1 and the activation of Gasdermin C. FIG. 10A shows that Vil^{CRE} Ifnlr1^{fl/fl} mice or WT mice were treated with either 50 mg kg⁻¹ day⁻¹ of rIFN-λ (rIFN-λ), or saline vehicle (Veh). Immunoblot analysis of the indicated proteins was performed. Each lane represents one mouse. GSDMC-2/-3 FL p50: full length 50 kDa GSDMC-2/-3; GSDMC-2/-3 CL p30: N-terminal 30 kDa cleaved protein; GSDMD FL p50: full length 50 kDa GSDMD; GSDMD CL p30: N-terminal 30 kDa cleaved protein; CASP-8 FL: full length CASP-8; CASP-8 p18: 18 kDa cleavage fragment. FIG. 10B shows the expression (defined by normalized transcript counts; Transcripts Per Kilobase Million [TPM] for PROTECT cohort and Reads Per Kilobase Million [RPKM] for RISK cohort) of the indicated genes was assessed in bulk RNA-seq data from the PROTECT (pediatric UC) and RISK (pediatric ileal CD) cohorts and comparisons made between control patients, uninflamed IBD and inflamed IBD patients. P-values are based on non-parametric t-testing between assessed groups (Wilcoxon test).

FIGS. 11A-11D shows how IFN-λ inhibits epithelial proliferation and survival in intestinal organoids in vitro. FIG. 11A shows that human duodenoids were seeded and treated (rIFN-λ), or not (Veh) with 200 ng/ml of human IFN-λ2 for 72 h. Cell viability was measured with CellTiter-Blue. Percentage of live organoids in rIFN-λ treated wells compared to Veh is depicted. FIG. 11B shows that mouse small intestinal organoids were grown for 6 days and then treated with mouse recombinant IFN-λ2 (rIFN-λ) at the indicated concentrations. Lgr5 relative mRNA expression is depicted. FIG. 11C shows that human duodenoids were seeded and treated with 200 ng/ml of IFN-λ2 for 72 hours. Organoids were pulsed with EdU for 6 hours. Organoids were stained for EdU incorporation. Mean fluorescence of EdU staining (left), relative organoid growth (right), are depicted. The relative growth of organoids is measured as the % of their area over untreated control organoids. FIG. 11D shows that colon organoids derived from WT or Zbp1^{-/-} mice were seeded and treated with 200 ng/ml of rIFN-λ and imaged at 24 hours, 48 hours and 72 hours. The number of formed organoids is depicted. FIG. 11C depicts box plots. Each dot represents a biological replicate. Median, range and interquartile range are depicted. FIG. 11D depicts a scatter box with bars. Each dot represents a biological replicate. Mean and SEM are depicted. FIG. 11C shows the statistics of the unpaired t-test. FIG. 11D shows the two-way ANOVA with Šidak correction for multiple comparisons. ns=not significant (p>0.05); *p<0.05; **p<0.01; ***p<0.001; ****p<0.0001.

FIGS. 12A-12C show the experimental design (FIG. 12A) immunofluorescent staining (FIG. 12B) and quantification of immunofluorescence (FIG. 12C) of small intestine organoids isolated from WT, Ripk1D138N mutant (kinase dead), Ripk3 KO, Ripk3/Caspase 8 dKO.

FIGS. 13A-13C show the experimental design (FIG. 13A) immunofluorescent staining (FIG. 13B) and quantification of immunofluorescence (FIG. 13C) of colon organoids isolated from WT or Zbp1^{ΔZα/ΔZα2}.

FIGS. 14A-14C show IFN-λ delays tissue repair during colitis. FIGS. 14A-14 show mice of indicated genotypes were administered 2.5% DSS in the drinking water for seven days. DSS was withdrawn on day 7 and mice were followed over time up to 12 days. FIG. 14A shows the weight change relative to baseline (left) and colon length (right) of Ifnlr1^{fl/fl} or Vil^{CRE}Ifnlr1^{fl/fl} mice 10 days after start of DSS treatment are depicted. FIGS. 14B-14C show colon lengths (FIG. 14B) and histology (FIG. 14C) were assessed on days 8, 10, and 12 after start of DSS treatment in Ifnlr1^{fl/fl} or Vil^{CRE}Ifnlr1^{fl/fl} mice.

FIG. 15 shows IFN-λ acts on IECs and delays tissue repair upon irradiation. WT or Ifnlr1^{-/-} mice were irradiated or left untreated (0 hours) and the number of intact crypts was quantified by histology after 24 hours or 72 hours.

FIGS. 16A-16G show IFN-λ controls the expression of Z-DNA Binding Protein 1 (ZBP1) and the activation of Gasdermin C (GSDMC) upon irradiation damage or during colitis.

FIGS. 16A-16B show plasmid DNA encoding full length human GSDMC (hGSDMC FL) or the N-terminal domain of human GSDMC (hGSDMC NT) was transfected in HEK293T cells at the indicated concentrations. FIG. 16A depicts the cell viability (left) and LDH release (right) were quantified 24 hours after transfection. FIG. 16B shows the plasma membrane integrity was assessed by monitoring Sytox Orange incorporation over time. FIGS. 16C-16D show small intestinal crypts were isolated from Vil^{CRE} Ifnlr1^{fl/fl} mice or WT mice irradiated (Irrad+) or not (Irrad−). FIG. 16C shows immunoblot analysis of the indicated proteins was performed. GSDMC-2/-3 FL p50: full length protein 50 kDa; GSDMC-2/-3 CL p30: N-terminal p30 cleaved protein; GSDMD FL p50: full length protein 50 kDa; GSDMD CL p30: N-terminal 30 kDa cleaved protein; CASP-8 cleaved protein p43/41; CL-CASP-8 p18: 18 kDa cleaved protein. FIG. 16D show graphs that indicate the densitometry quantification of the band intensity from the image depicted in (FIG. 16C) and each dot represents one lane. FIGS. 16E and 16G show colonocytes were isolated on day 10 (FIG. 16E) or days 8, 10, and 12 (FIG. 12G) from Vil^{CRE} Ifnlr1^{fl/fl} mice or WT mice treated with DSS as in FIG. 14A (DSS+) or not (DSS−). Immunoblot analysis of the indicated proteins was performed. GSDMC-2/-3 FL p50: full length protein 50 kDa; GSDMC-2/-3 CL p30: N-terminal p30 cleaved protein; CL-CASP-8 p43/41: CASP-8 cleaved protein p43/41; CL-CASP-8 p18: 18 kDa cleaved protein; CASP-8: Full length CASP-8. FIG. 16F are e graphs indicating the densitometry quantification of the band intensity from the image depicted in (FIG. 16E) and each dot represents one mouse. FIG. 16A-16B shoe the mean and SEM of 3 independent experiments. In FIGS. 16C-16F each lane represents one mouse. Representative data of 3 independent experiments is depicted. Statistics: FIG. 16A used a one way ANOVA with Dunnett correction for multiple comparisons (FIG. 16B) area under curve (AUC) was calculated for each treatment and One way ANOVA with Dunnett correction for multiple comparisons was performed on AUC values and variance. FIGS. 16D and 16F used two-way ANOVA with Tukey correction for multiple comparisons. ns=not significant (p>0.05); *p<0.05; **p<0.01; ***p<0.001; ****p<0.0001.

FIGS. 17A-17B show the ZBP-1/Casp-8/GSDMC pathway is active in IBD patients. FIG. 17A shows western blot analyses of the indicated proteins were performed on cell lysates of human colon biopsies derived from ascending or transverse colons of healthy controls (HC), or of Crohn's disease (CD) or ulcerative colitis (UC) patients. 3 representative samples per group are shown. FIG. 17B shows the box plots indicate the densitometry quantification of the band intensity from western blot analyses images for 7 HC and 8 CD or UC patients. Each dot represents one sample. Median, range and interquartile range are depicted. Statistics: FIG. 17B used one-way ANOVA, with Tukey correction for multiple comparisons. ns=not significant (p>0.05); *p<0.05; **p<0.01; ***p<0.001.

FIG. 18A-18F show IFN-λ drives pyroptosis of IECs. FIG. 18A shows small intestinal organoids were treated as in (FIG. 6A) for 24, 48 and 72 hours. Immunoblot analysis of the indicated proteins was performed. GSDMC-2/-3 FL p50: full length protein 50 kDa; GSDMC-2/-3 CL p30: N-terminal p30 cleaved protein; CL-CASP-8 p43/41: 43 and 41 kDa cleaved protein; CL-CASP-8 p18: 18 kDa cleaved protein. Representative blot of three independent experiments. FIG. 18B depicts the experimental scheme for the establishment of 2D Air Liquid Interface (ALI) organoid cultures and modeling of damage and repair responses. Organoids were seeded in transwells and grown to confluence. The apical side was then exposed to air up to 14 days, which favored differentiation of a homeostatic monolayer (Homeostasis). Organoids were then submerged for 7 days to induce damage responses (Re-sub/Damage). After 7 days they were re-exposed to air to stimulate repair responses (Repair). Organoids were then treated at different steps of the culture with 200 ng/ml of rIFN-λ for 3 days. FIGS. 18C and 18F show organoids treated during Re-sub/Damage (FIG. 18C, FIG. 18D) or Repair (FIG. 18E, 18F) as described in (FIG. 18B) were pulsed with Zombie dye for 30 minutes to mark dead cells and stained for Ki67 to mark proliferating cells), CL-Casp-8 and DAPI. Representative image of 3 independent experiments. FIGS. 18D and 18F show the quantification of the staining depicted in (FIGS. 18C and 18E) respectively. FIGS. 18D and 18F show (the mean and SEM of 3 independent experiments are depicted. FIGS. 18D and 18F are scatter plots. Each square represents an independent experiment. Statistics: FIGS. 18D and 18F used a two-way ANOVA with Šidak correction for multiple comparison. ns=not significant (p>0.05); *p<0.05; **p<0.01; ***p<0.001; ****p<0.0001. Scale bars: (E, G) 20 μm.

FIGS. 19A-19J show the ZBP-1/Casp-8/GSDMC axis mediates IEC pyroptosis in response to damage and IFN-λ encounter. FIG. 19A-19B show organoids transduced with non-targeting gRNA (NT) or a gRNA targeting Zbp1 (Zbp1^{KD}) were seeded in transwells and grown to confluence. The apical side was then exposed to air for 7 days then re-submerged in presence of 200 ng/ml of rIFN-λ for 48 hours. Organoids were pulsed with Zombie dye for 30 minutes then stained with CL-Casp-8 and DAPI). FIG. 19A shows the quantification of Zombie-positive cells (left) and CL-Casp-8-positive cells (right) are depicted. FIG. 19B shows representative images of 3 independent experiments are depicted. FIG. 19C shows small intestine NT or Zbp1^{KD} organoids that were seeded and allowed to grow for 48 hours. Organoids were then treated with 200 ng/ml of rIFN-λ in the presence of 1 g/ml propidium iodide (PI) and imaged every 12 hours over 48 hours. Percentage of live organoids was calculated as percentage of PI⁻ organoids over the total number of organoids in each well. FIG. 19D-19G show organoids were treated with 200 ng/ml of rIFN-λ or left untreated (Veh) either during Re-sub (FIGS. 19D and 19E) or during Repair (FIGS. 19F and 19G) with rIFN-λ as described in (FIG. 18B). Additionally, organoids were treated with 40 M of Z-VAD-FMK (Z-VAD) or left untreated (NT) as indicated. Organoids were pulsed with Zombie dye for 30 minutes, and stained with Ki67, CL-Casp-8, and DAPI. FIGS. 19D and 19F show the quantification of Zombie-positive cells (left), CL-Casp-8-positive cells (middle 1, only FIG. 19D), and Ki67-positive cells (right) are depicted. FIGS. 19E and 19G are representative images of 3 independent experiments are depicted. FIGS. 19H and 19I showCas9 expressing organoids transduced with non-targeting gRNA (NT) or gRNAs targeting Gsdmc 2 and 3 (Gsdmc2,3^{KD}) were treated during Re-sub as described in (FIG. 19A), pulsed with Zombie dye for 30 minutes then stained with CL-Casp-8 and DAPI. FIG. 19H shows the quantification of Zombie-positive cells (left) and CL-Casp-8-positive cells (right). FIG. 19I shows representative images of 3 independent experiments are depicted. FIG. 19J shows NT or Gsdmc2,3^{KD} organoids were treated as in (FIG. 19C) and percentage of live organoids over time is depicted. Statistics: FIGS. 19A, D, F, and H used two-way ANOVA with Šidak correction for multiple comparisons. Each dot or square represents an independent experiment. ns=not significant (p>0.05); *p<0.05; **p<0.01; ***p<0.001; ****p<0.0001. The scale bars in FIGS. 19B, 19E, 19G, and 19I are 20 m.

FIGS. 20A-20E show IFN-λ delays tissue repair during colitis. FIGS. 20A-20E show mice of the indicated genotypes were administered 2.5% DSS in the drinking water for seven days. DSS was withdrawn on day 7 and mice were followed over time up to 12 days. Relative mRNA levels of Ifnl2 and Ifnl3 (FIG. 20A), and Ifit1 (FIG. 20B) were measured in whole colon homogenates at the indicated time points. FIG. 20C shows the weight change relative to baseline (left) and colon length (right) of Ifnlr1^{fl/fl} or MRP8^{CRE}Ifnlr1^{fl/fl} mice 10 days after start of DSS treatment are depicted. FIG. 20D shows the weight change from baseline was assessed over time in Vil^{CRE}Ifnlr1^{fl/fl} or Ifnlr1^{fl/fl} littermates. FIG. 20D are the colon histology scores at days 8, 10, and 12 after start of DSS treatment are depicted. FIGS. 20A, 20B, and 20C are box plots. Each dot represents one mouse. Median, range and interquartile range are depicted. FIGS. 20D and 20E show the mean with SEM. Statistics: FIGS. 20A, 20B, 20D, and 20E used a one-way ANOVA with Šidak correction for multiple comparisons. FIG. 20C used a Mann-Whitney test. ns=not significant (p>0.05); *p<0.05; **p<0.01; ***p<0.001; ****p<0.0001.

FIGS. 21A-21C show IFN-λ acts on IECs and delays tissue repair upon irradiation. FIG. 21A show WT mice received 11 Gy ionizing radiation, with lead shielding of the upper body, relative mRNA levels of Ifnl2 and Ifnl3 were measured in total colon homogenates. FIG. 21A-21C show WT or Ifnlr1^{-/-} mice received 11 Gy ionizing radiation with lead shielding of the upper body and were treated with either 50 μg kg⁻¹ day⁻¹ of rIFN-λ (rIFN-λ), or saline vehicle (Veh). Tissue repair in the small intestine was evaluated by histology 48 hours and 96 hours after irradiation. FIG. 21B shows the number of intact crypts per histological section (Crypts/section). FIG. 21C shoes the representative histology images. FIG. 21A is a scatter plot. FIG. 21B shoes the mean with SEM. FIGS. 21A and 21D used a one-way ANOVA with Dunnett correction for multiple comparisons. FIG. 21B used a two-way ANOVA with Šidak correction for multiple comparisons.

FIGS. 22A-22E show IFN-λ controls the expression of Z-DNA Binding Protein 1 (ZBP1) and the activation of Gasdermin C (GSDMC) upon irradiation damage or during colitis. FIG. 22A is the immunoblot analysis of the indicated organs from two WT mice for GSDMC expression was performed. FIG. 22B shows Gsmdc1-4 mRNA expression was analyzed by qPCR in organs isolated from WT mice. FIG. 22C shows the immunoblot analysis of GSDMC expression in colonic cell populations (intestinal epithelial cell (IEC) and lamina propria (LP) cells) isolated from WT mice at homeostasis. Each lane represents one mouse. FIG. 22D shows HEK293T cells were transfected with the pRetroX TetOne3G-eGFP plasmid harboring Flag-tagged N-terminal fragment of human GSDMC (hGSDMC NT) and treated or not with the indicated concentrations of doxycycline (DOX). Immunoblot analysis of Flag tag expression was performed and cell viability as well as LDH release were determined 24 hours post DOX treatment. pRetroX TetOne3G-eGFP plasmid harboring mouse GSDMD N-terminal fragment (MsGSDMD NT) was used as positive control. FIG. 22E shows WT mice were treated with DSS for 7 days at the indicated concentrations. DSS was withdrawn on day 7. Colonocytes were isolated 8 days after start of DSS treatment. Immunoblot analysis of the indicated proteins was performed (upper panel). GSDMC-2/-3 FL p50: full length 50 kDa; GSDMC-2/-3 CL p30: p30 cleaved GSDMC-2/-3. Densitometry quantification of the band intensity from the image. Each lane represents one mouse. FIG. 22B shows the mean and SD from one of two independent experiments are depicted. FIG. 22D is the mean and SD of two independent experiments performed in triplicate are depicted. FIG. 22E show scatter plots are depicted. Each dot represents one mouse. Statistics: FIGS. 22D and 22E used One-way ANOVA with Dunnett correction for multiple comparisons. ns=not significant (p>0.05); *p<0.05; **p<0.01; ***p<0.001; ****p<0.0001.

FIGS. 23A-23I show the ZBP-1/Casp-8/GSDMC pathway is active in IBD patients. FIG. 23 shows the expression (defined by normalized transcript counts; Reads Per Kilobase Million [RPKM]) of GSDMC (FIG. 23A), ZBP1 (FIG. 23B), and CASP8 (FIG. 23C) from published RNAseq data for the PROTECT (pediatric UC) cohort. FIGS. 23D-23F show expression (defined by Transcripts Per Kilobase Million [TPM]) of GSDMC (FIG. 23D), ZBP1 (FIG. 23E), and CASP8 (FIG. 23F) from published RNAseq data RISK (pediatric ileal CD) cohort. FIG. 23G-23I show RNA sequencing on colon biopsies from control patients, IBD patients with inactive disease, and IBD patients with active disease (see Materials and Methods). Square symbols represent controls, round symbols represent ulcerative colitis (UC) patients, and triangles represent Crohn's disease patients (CD). Expression of GSDMC (FIG. 23G), ZBP1 (FIG. 23H), CASP8 (FIG. 23I) expressed as normalized log 2 count is depicted. FIGS. 23A-23F are violin plots, FIGS. 23G-23I are box plots with median, range, and interquartile range are depicted. In FIGS. 23A-23I each symbol represents one patient. Comparisons were made between control and uninflamed IBD patients and inflamed IBD patients. P-values are based on the Kruskall Wallis test with Dunn correction for multiple comparisons.

FIG. 24A-24D show IFN-λ drives pyroptosis of IECs. FIG. 24A-24B show mouse small intestinal organoids were seeded from freshly isolated crypts and allowed to grow for 48 h. Organoids were then treated with 20 ng/ml or 200 ng/ml of rIFN-λ. FIG. 24A depicts an immunoblot analysis of the indicated proteins was performed. Representative blot of three independent experiments (FIG. 24B) Organoids were cultured in the presence of 1 μg/ml propidium iodide (PI) and imaged every 12 hours over 72 hours. Percentage of live organoids was calculated as the percentage of PI⁻ organoids over the total number of live organoids in each well. FIG. 24C shows mouse organoids seeded in transwells and grown to confluence as in FIG. 18B. The apical side was then exposed to air up to 14 days and treated with 200 ng/ml of rIFN-λ for 3 days. Organoids were pulsed with Zombie dye for 30 minutes, and stained with Ki67, CL-Casp-8 and DAPI. Representative image of 3 independent experiments and quantification of the staining is depicted. FIG. 24I shows mouse organoids seeded in transwells and grown to confluence were treated during re-submersion with 200 ng/ml of rIFN-λ or rIFNλ for 3 days. Organoids were pulsed with Zombie dye for 30 minutes, and stained with Ki67, CL-Casp-8 and DAPI. Representative image of 3 independent experiments and quantification of the different stainings are depicted. FIGS. 24C-24D are scatter plots with bars. Each dot or square represents an independent experiment. Mean and SEM are depicted. Statistics: FIGS. 24C-24D used two-way ANOVA with Šidak correction for multiple comparisons. ns=not significant (p>0.05); *p<0.05; **p<0.01; ***p<0.001; ****p<0.0001. The scale bars in FIGS. 24C and 24D are 20 μm.

FIGS. 25A-25H show the ZBP-1/Casp-8/GSDMC axis mediates IEC pyroptosis in response to damage and IFN-λ encounter. FIG. 25A shows Cas9 expressing organoids transduced with non-targeting gRNA (NT) or a gRNA targeting Zbp1 (Zbp1^{KD}) were grown for 6 days and then treated with 200 ng/ml of rIFN-λ for 48 hours. Immunoblot analysis of the indicated proteins was performed. FIGS. 25B-25C shows small intestine organoids were seeded in transwells and grown to confluence. Organoids were treated concomitantly to re-exposure to air either with 200 ng/ml of rIFN-λ alone (rIFN-λ) or with rIFN-λ together with Z-IETD. Organoids were pulsed with Zombie dye for 30 minutes, and stained with Ki67, CL-Casp-8 and DAPI. Representative images of 3 independent experiments (FIG. 25B) and quantification of the different stainings are depicted (FIG. 25C). FIGS. 25D-25G show small intestine organoids were seeded from freshly isolated crypts from WT (FIGS. 25D, 25E, 25G) or WT and Mlkl^{-/-} (FIG. 25F) mice and allowed to grow for 48 hours. Organoids were then treated with 200 ng/ml of rIFN-λ. FIGS. 25D, 25E, and 25G show treatment with rIFN-λ was performed together with the pan-caspase inhibitor Z-VAD-FMK (Z-VAD; 40 uM) (FIG. 25D), the caspase-8 specific inhibitor Z-IETD-FMK (Z-IETD; 40 uM) (FIG. 25E), or the inhibitor of gasdermin D pore formation Disulfiram (10 μM or 50 μM) (FIG. 25G). FIGS. 25D-25G show organoids were grown in the presence of 1 μg/ml propidium iodide (PI) and imaged every 12 hours over 72 hours. Percentage of live organoids was calculated as percentage of PI⁻ organoids over the total number of organoids in each well. Statistical comparison between “rIFN-λ” and “Veh” are depicted as red (*), comparison between “rIFN-λ+Z-VAD” (FIG. 25D) or “rIFN-λ+Z-IETD” (FIG. E) and “rIFN-λ” are depicted as grey ($). The area under the curve (AUC) was calculated for each treatment (right panel). FIG. 25H shows the immunoblot analysis of the indicated proteins was performed on extracts from Cas9 expressing organoids transduced with non-targeting gRNA (NT) or 2 gRNAs targeting both Gsdmc2,3 (Gsdmc2,3^{KD}), or non-transduced (−) organoids. Statistics: FIGS. 25D-25F used two-way ANOVA with Tukey correction for multiple comparisons. FIGS. 25C, 25D, 25E, and 25G used Two-way ANOVA with Šidak correction for multiple comparisons. ns=not significant (p>0.05); * p<0.05; ** or $$ p<0.01; *** p<0.001; **** p<0.0001. The scale bars in FIG. 25B are 20 μm.

### DETAILED DESCRIPTION OF CERTAIN EMBODIMENTS

Aspects of the present disclosure are based on the discovery that interferon signaling, specifically that of interferon lambda (IFNL, IFN-λ), also referred to as type III interferon, not only mediates inflammation in intestinal epithelia during inflammatory diseases, but also impairs tissue repair by upregulating a variety of key factors that promote cell death by pyroptosis and necroptosis. Although impaired tissue repair has long been recognized as a core therapeutic challenge for the treatment of intestinal inflammatory diseases, such as inflammatory bowel diseases (IBDs) in particular (e.g., Crohn's disease (CD), ulcerative colitis (UC)), the IFNL signaling axis had not previously been demonstrated to play such a central role in these pathologies.

### Interferon Lambda (IFNL) Signaling in Intestinal Epithelia

As used herein, the term “IFNL” refers to any protein expressed from a gene encoding IFNL, e.g., IFNL-1 (interleukin 29 (IL-29)), IFNL-2 (interleukin 28A (IL-28A)), IFNL-3 (interleukin 28B (IL-28B); interleukin 28C (IL-28C)), or IFNL-4 (IFNAN), each of which are expressed from a different genetic locus within the Homo sapiens genome. In some embodiments, an IFNL protein contemplated herein is IFNL-1 (NCBI Reference Sequence: NP_742152.1; Gene ID: 282618). In some embodiments, an IFNL protein contemplated herein is IFNL-2 (NCBI Reference Sequence: NP_742150.1; Gene ID: 282616). In some embodiments, an IFNL protein contemplated herein is IFNL-3 (NCBI Reference Sequence: NP_742151.2; NCBI Reference Sequence: NP_001333866.1; Gene ID: 282617). In some embodiments, an IFNL protein contemplated herein is IFNL-4 (NCBI Reference Sequence: NP_001263183.2; Gene ID: 101180976). In some embodiments, an IFNL protein contemplated herein is an IFNL protein (e.g., IFNL-1, IFNL-2, IFNL-3, or IFNL-4) that is endogenously expressed by a subject, e.g., a subject having an inflammatory disease, such as an IBD (e.g., CD, UC). In some embodiments, an IFNL protein contemplated herein is an IFNL protein (e.g., IFNL-1, IFNL-2, IFNL-3, or IFNL-4) that is endogenously expressed by a human subject. In some embodiments, an IFNL protein contemplated herein is an IFNL protein endogenously expressed by a non-human subject, such as a non-human mammal, which is homologous to human IFNL (e.g., IFNL-1, IFNL-2, IFNL-3, IFNL-4).

As used herein, the term “IFNLR” refers to any protein expressed from a gene encoding an IFNL receptor protein, e.g., interferon lambda receptor 1 (IFNLR1), also known as CRF2/12, interferon lambda receptor (IFNLR), interleukin 28 receptor 1 (IL-28R1), interleukin 28 receptor A (IL28RA), and LICR2. In some embodiments, an IFNLR protein contemplated herein is IFNLR1 (NCBI Reference Sequence: NP_734464.1; NCBI Reference Sequence: NP_775087.1; NCBI Reference Sequence: NP_775088.1; Gene ID: 163702). In some embodiments, an IFNLR protein contemplated herein is an IFNLR protein (e.g., IFNLR1) that is endogenously expressed by a subject, e.g., a subject having an inflammatory disease, such as an IBD (e.g., CD, UC). In some embodiments, an IFNLR protein contemplated herein is an IFNLR protein (e.g., IFNLR1) that is endogenously expressed by a human subject. In some embodiments, an IFNLR protein contemplated herein is an IFNLR protein endogenously expressed by a non-human subject, such as a non-human mammal, which is homologous to human IFNLR (e.g., IFNLR1).

Without wishing to be bound by theory, recent studies have proposed various and at times conflicting activities for IFNL signaling in regard to intestinal epithelial damage. Initially, IFNL was demonstrated to limit inflammation during colitis by reducing the tissue damaging activity of neutrophils (see, e.g., Broggi A, et al., “IFN-λ suppresses intestinal inflammation by non-translational regulation of neutrophil function.” Nat Immunol. 2017; 18(10):1084-1093). This phenomenon is primarily explained by the expression pattern for IFNL receptor (IFNLR), which is restricted to the cell surface of neutrophils and epithelial cells and is required to propagate intracellular IFNL signaling cascades, unlike type I interferons such as interferon alpha (IFNA, IFN-λ) which act systemically and drive inflammatory activities. The role of IFNL for the reconstitution of intestinal epithelia following tissue damage has been less clear, however. IFNL has been proposed to facilitate the proliferation of intestinal epithelial cells via signal transducer and activator of transcription 1 (STAT1) signaling (see, e.g., Chiriac M T, et al. “Activation of Epithelial Signal Transducer and Activator of Transcription 1 by Interleukin 28 Controls Mucosal Healing in Mice With Colitis and Is Increased in Mucosa of Patients With Inflammatory Bowel Disease.” Gastroenterology. 2017; 153(1):123-138) and to enhance the integrity of the intestinal mucosal layer during graft versus host disease (Henden A S, et al., “IFN-λ therapy prevents severe gastrointestinal graft-versus-host disease.” Blood. 2021 26; 138(8):722-737). On the other hand, however, IFNL and/or IFNLR were also found to be upregulated in IBD patients (see, e.g., Chiriac M T, et al. “Activation of Epithelial Signal Transducer and Activator of Transcription 1 by Interleukin 28 Controls Mucosal Healing in Mice With Colitis and Is Increased in Mucosa of Patients With Inflammatory Bowel Disease.” Gastroenterology. 2017; 153(1):123-138; Gunther C, et al. “Interferon Lambda Promotes Paneth Cell Death Via STAT1 Signaling in Mice and Is Increased in Inflamed Ieal Tissues of Patients With Crohn's Disease.” Gastroenterology. 2019; 157(5):1310-1322). Systemic and prolonged overexpression of IFNL in mice also was observed to increase the death of Paneth cells, a group of cells that can facilitate epithelial cell regeneration by acquiring stem-like features (Schmitt M, et al., “Paneth Cells Respond to Inflammation and Contribute to Tissue Regeneration by Acquiring Stem-like Features through SCF/c-Kit Signaling.” Cell Rep. 2018; 24(9):2312-2328), and by regulating the balance of epithelial growth factors in the stem cell niche (Sato T, et al. “Paneth cells constitute the niche for Lgr5 stem cells in intestinal crypts.” Nature. 2011; 469(7330):415-8). Separately, during viral infection, expression of IFNL in the lower respiratory tract of COVID-19 patients was shown to be associated with increased apoptosis and decreased proliferation and is characteristic of sever-to-critical SARS-CoV-2-infection outcomes (Broggi A, et al., “Type III interferons disrupt the lung epithelial barrier upon viral recognition.” Science. 2020; 369(6504):706-712), yet it remained unclear whether IFNL has a similar role in intestinal epithelial cells during inflammatory disease states, especially in the context of non-viral inflammatory diseases.

Subsequent studies now demonstrate that IFNL acts through a previously undiscovered signaling axis, wherein activation of IFNLR by IFNL on the surface of inflamed epithelial cells culminates in activation of Z-DNA binding protein 1 (ZBP1) and factors involved in activity of the PANoptosome, a multi-subunit complex that promotes apoptosis, pyroptosis, and/or necroptosis pathways (“PANoptosis”) in intestinal epithelial cells. For example, activation of IFNLR was specifically demonstrated to stimulate caspase-8-dependent cleavage of gasdermin C (GSDMC), thereby inducing pyroptosis of intestinal epithelial cells and delaying restitution of intestinal epithelia following tissue damage. Interferon stimulated genes include Z-DNA-binding protein 1 (ZBP1), Viperin (RSAD2), and gasdermin C family (e.g., Gsdmc2 and Gsdmc3). ZBP1 comprises a z-nucleic acid binding site. While this previously unrecognized IFNL signaling axis is unexpected, considering the prevailing notion in the field that IFNL protects intestinal epithelia during inflammation by directly interfering with the tissue-damaging activities of neutrophils, its discovery could enable the development of new techniques for treating intestinal inflammatory diseases (e.g., IBD). Thus, various agents are contemplated herein which are of use for reducing the activity of the IFNL signaling axis in intestinal epithelial cells. Said agents may interfere with IFNL signaling by inhibiting the function of IFNL and/or IFNLR directly or may interfere with IFNL signaling by inhibiting downstream components of IFNL signaling, such as, but not limited to, factors involved with the activation of PANoptosis. Methods of administering these agents to subjects having intestinal inflammatory diseases (e.g., IBD) may be utilized to treat intestinal inflammatory diseases by reducing inflammation and cell death caused by IFNL signaling, while promoting the repair of damaged tissues.

### Administration of Agents for Inhibiting IFNL Signaling

The present disclosure provides methods for the administration of an agent that is sufficient to inhibit interferon lambda (IFNL) signaling in intestinal epithelial cells, or a composition thereof (e.g., a pharmaceutical composition), to a subject. In some embodiments, a method is provided for treating or preventing a disease in a subject by administering an agent that is sufficient to inhibit interferon lambda (IFNL) signaling in intestinal epithelial cells, or a composition thereof (e.g., a pharmaceutical composition), to a subject. In some embodiments, a therapeutically effective amount of the agent is administered to the subject such that the method results in the treatment or prevention of a disease in the subject. In some embodiments, the disease is an inflammatory disease present in intestinal epithelia of the subject, such as, but not limited to, an inflammatory bowel disease (IBD), such as Crohn's disease (CD) or ulcerative colitis (UC). In some embodiments, the disease is another inflammatory disease affecting the intestinal epithelia that is generally known in the art, such as an autoimmune disease or another disease that has been associated with pathogenic inflammation, such as irritable bowel syndrome (IBS).

As used herein, the terms “administer,” “administering,” or “administration” refer to implanting, absorbing, ingesting, injecting, inhaling, or otherwise introducing an agent described herein, or a composition thereof (e.g., a pharmaceutical composition), in or on a subject. As used herein, the term “treatment,” “treat,” and “treating” refers to the application or administration of an agent described herein, or a composition thereof (e.g., a pharmaceutical composition), to a subject in need thereof for the purpose of reducing the severity of a disease (e.g., IBD) in the subject. A “subject in need thereof” refers to an individual that has a disease, a symptom of the disease, or a predisposition toward the disease. A method for treating a disease may encompass administering to a subject an agent described herein, or a composition thereof (e.g., a pharmaceutical composition) with the intention to cure, heal, alleviate, relieve, alter, remedy, ameliorate, improve, or affect the disease, a symptom of the disease, or predisposition toward the disease in the subject. A method for treating a disease may encompass prophylaxis, wherein an agent is administered to the subject for the purpose of preventing development of the disease, for example, in a subject that is not known to have the disease, but may develop or be at risk of developing the disease in the future.

As used herein, a “therapeutically effective amount” or “effective amount” refers to the amount of an agent (e.g., an agent described herein) that is sufficient to elicit the desired biological response in the subject, for example, alleviating one or more symptoms of the disease (e.g., IBD). A therapeutically effective amount may be an amount that is either administered to the subject alone or in combination with one or more other agents. Effective amounts vary, as recognized by those skilled in the art, depending on such factors as the desired biological endpoint, the pharmacokinetics of the administered agent, the particular condition or disease being treated, the severity of the condition or disease, the individual parameters of the subject, including age, physical condition, size, gender and weight, the duration of the treatment, the nature of any other concurrent therapy, the specific route of administration, and like factors that are within the knowledge and expertise of the health practitioner to determine. These factors are well known to those of ordinary skill in the art and can be addressed with no more than routine experimentation. It is generally preferred that a maximum dose of the individual agents described herein (e.g., an agent described herein) or any combinations thereof to be used is at most the highest dose that can be safely administered to the subject according to sound medical judgment. Preferably, an effective dose is lower than the highest dose that can be safely administered to the subject. It will be understood by those of ordinary skill in the art, however, that a subject or health practitioner may select a lower dose (e.g., the minimum effective dose) in order to mitigate any potential risks of treatment, such as side effects of the treatment.

In some embodiments, for an adult subject of normal weight, doses ranging from about 0.01 to 1000 mg/kg of an agent (e.g., an agent described herein) may be administered. In some embodiments, the dose is between 1 to 200 mg. The particular dosage regimen, i.e., the dose, timing, and repetition, will depend on the particular subject and that subject's medical history, as well as the properties of the agent (such as the pharmacokinetics of the agent) and other consideration well known in the art.

Treating a disease (e.g., IBD) may include delaying the development or progression of the disease or reducing disease severity. Treating the disease does not necessarily require curative results. As used herein, “delaying” the development of a disease means to defer, hinder, slow, retard, stabilize, and/or postpone progression of the disease in a subject. Delaying the progression of a disease may include delaying or preventing the spread of a disease occurring in a subject, such as, for example, delaying or preventing the spread of an IBD occurring in a subject to intestinal epithelial tissues not yet affected by the IBD. This delay can be of varying lengths of time, depending on the history of the disease and/or individuals being treated. A method that delays the development of a disease, or delays the onset of the disease, is a method that reduces probability of developing one or more symptoms of the disease in a given time frame and/or reduces extent of the symptoms in a given time frame, as compared to the absence of such a method. Comparisons are typically based on clinical studies, using a number of subjects sufficient to give a statistically significant result.

The “development” or “progression” of a disease (e.g., IBD) refers to initial manifestations and/or ensuing progression of the disease in a subject. Development of a disease can be detectable and assessed using standard clinical techniques as well known in the art. However, development also refers to progression that may be undetectable. For purpose of this disclosure, development or progression may refer to the development or progression of symptoms of a disease. The term “development” includes the occurrence, recurrence, and onset of a disease. As used herein “onset” or “occurrence” of a disease includes the initial onset of a disease, as well as recurrence of the disease (i.e., in a subject who has had the disease previously).

In some embodiments, the agent with which a subject is treated comprises an antibody, a small molecule, a nucleic acid, or a gene editing agent.

In some embodiments, the agent is an antibody (e.g., a monoclonal antibody) that binds to IFNL or IFNLR. In some embodiments, the antibody preferentially binds to IFNL or IFNLR (i.e., the antibody may also bind to other species, such as another interferon or interferon receptor, but with lower affinity as compared to IFNL or IFNLR). In some embodiments, the antibody specifically binds to IFNL or IFNLR. In some embodiments, the antibody binds to IFNL or IFNLR on the cell surface of intestinal epithelial cells of the subject. In some embodiments, binding of the antibody to IFNL or IFNLR (e.g., on the surface of intestinal epithelial cells of the subject) results in inhibition of IFNL or IFNLR (e.g., in intestinal epithelial cells of the subject). In some embodiments, binding between the antibody and IFNL or IFNLR results in inhibition of the activity of IFNL or IFNLR (e.g., IFNL signaling activity) in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the agent is a small molecule (e.g., a small molecule inhibitor) that binds to interferon lambda receptor (IFNLR). In some embodiments, the small molecule preferentially binds to IFNLR (i.e., the small molecule may also bind to other species, such as another interferon receptor, but with lower affinity as compared to IFNLR). In some embodiments, the small molecule specifically binds to IFNLR. In some embodiments, the small molecule binds to IFNLR on the cell surface of intestinal epithelial cells of the subject. In some embodiments, binding between the small molecule and IFNLR results in inhibition of the activity of IFNLR (e.g., IFNL signaling activity) in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the agent is a small molecule that binds to a Janus kinase (JAK) protein that is bound to interferon lambda receptor (IFNLR) on the cell surface of intestinal epithelial cells of the subject. Without wishing to be bound by theory, JAK proteins, such as Janus kinase 2 (JAK2), propagate IFNL signaling that results from binding between IFNL and IFNLR on the cell surface, resulting in inflammation and cell death. In some embodiments, the JAK protein is JAK2. In some embodiments, the JAK protein does not bind to other interferon receptors. For example, in some embodiments, the small molecule does not bind to a JAK protein that is bound to interferon alpha receptor (IFNAR) on the cell surface of intestinal epithelial cells of the subject. In some embodiments, the small molecule inhibits the activity of the JAK protein (e.g., JAK2) in intestinal epithelial cells of the subject. In some embodiments, binding between the small molecule and the JAK protein (e.g., JAK2) bound to IFNLR on the cell surface of intestinal epithelia of the subject results in inhibition of the activity of the JAK protein (e.g., IFNL signaling activity) in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the agent is a small molecule inhibits caspase activity in the subject. In some embodiments, the caspase inhibitor is a pan-caspase inhibitor. In some embodiments, the pan-caspase inhibitor is Z-VAD-FMK. In some embodiments, the caspase inhibitor is a caspase 8 (Casp-8) inhibitor. In some embodiments, the Casp-8 inhibitor is Z-IETD-FMK. In some embodiments, administering the caspase inhibitor to the subject results in inhibition of caspase activity (e.g., IFNL-dependent caspase-8 activation that drives cell death) in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the small molecule binds to a factor (e.g., a protein) of PANoptosis signaling in intestinal epithelial cells of the subject. Without wishing to be bound by theory, activation of IFNL signaling in the context of an inflammatory intestinal epithelial disease can result in subsequent activation of factors comprising the PANoptosome, a multi-subunit complex that promotes apoptosis, pyroptosis, and/or necroptosis pathways in intestinal epithelial cells. Inhibition of one or more factors required for PANoptosome function will therefore prevent the activation of apoptosis, pyroptosis, and/or necroptosis pathways as a result of IFNL signaling in intestinal epithelia. A factor of PANoptosis signaling may be any factor that is known to be involved in signaling for apoptosis, pyroptosis, and/or necroptosis. A factor of PANoptosis signaling may be involved in a specific cell death signaling pathway. For example, a factor of PANoptosis signaling specifically involved in necroptosis may be mixed lineage kinase domain-like pseudokinase (MLKL), dynamin-related protein 1 (Drp1), or PGAM family member 5 (PGAM-5), while a factor of PANoptosis signaling specifically involved pyroptosis may be caspase-1 or Gasdermin D. In some embodiments, the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein. In some embodiments, the caspase protein is caspase-8, caspase-3, caspase-7, or caspase-1. In some embodiments, the gasdermin protein is Gasdermin C or Gasdermin D. In some embodiments, a small molecule inhibiting a factor of PANoptosis signaling is generally known in the relevant art. For example, in some embodiments, the factor of PANoptosis signaling is RIPK1 and the small molecule is necrostatin. In some embodiments, binding between the small molecule and the factor of PANoptosis signaling results in inhibition of the activity of the factor of PANoptosis signaling (e.g., IFNL signaling activity) in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the nucleic acid is a small interfering RNA (siRNA) or a short hairpin RNA (shRNA). In some embodiments, the nucleic acid inhibits the expression (e.g., protein expression) of IFNLR, a JAK protein, or a factor of PANoptosis signaling in intestinal epithelial cells of the subject. In some embodiments, the JAK protein is JAK2. In some embodiments, the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein. In some embodiments, the caspase protein is caspase-8, caspase-3, caspase-7, or caspase 1. In some embodiments, the gasdermin protein is Gasdermin C or Gasdermin D. In some embodiments, the nucleic acid inhibits the expression of IFNLR, the JAK protein, or the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the gene editing agent comprises a zinc finger nuclease (ZFN), a transcription activator-like effector nuclease (TALEN), or a clustered regularly interspaced short palindromic repeat (CRISPR)-Cas-associated nuclease (CRISPR/CAS), or another gene editing agent that is generally known in the relevant art. In embodiments where the gene editing agent comprises CRISPR/CAS, the gene editing agent further comprises a short guide RNA (sgRNA) that is complementary to a gene encoding interferon lambda receptor (IFNLR), a Janus kinase (JAK) protein, or a factor of PANoptosis signaling in intestinal epithelial cells of the subject. In some embodiments, the gene editing agent binds to and modifies (e.g., via endonuclease activity) a gene encoding interferon lambda receptor (IFNLR), a Janus kinase (JAK) protein, or a factor of PANoptosis signaling in intestinal epithelial cells of the subject. In some embodiments, the JAK protein is JAK2. In some embodiments, the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein. In some embodiments, the caspase protein is caspase-8, caspase-3, caspase-7, or caspase 1. In some embodiments, the gasdermin protein is Gasdermin C or Gasdermin D. In some embodiments, the gene editing agent reduces the expression of IFNLR, the JAK protein, or the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

In some embodiments, the agent (e.g., an antibody, a small molecule, a nucleic acid, or a gene editing agent) administered to a subject for the purpose of treating an inflammatory disease affecting intestinal epithelia of the subject (e.g., IBD) further comprises a delivery agent. As defined herein, a “delivery agent” refers to any moiety that enhances the delivery of an agent (e.g., an antibody, a small molecule, a nucleic acid, or a gene editing agent) to a desired organ or tissue (e.g., intestinal epithelia), as compared delivery of the agent when administered alone. In some embodiments, the delivery agent is a peptide, an antibody, a liposome, or a viral particle, or another delivery agent known in the art that is generally suitable for intestinal delivery. In some embodiments, the delivery agent binds specifically to the cell surface of intestinal epithelial cells of the subject. In some embodiments, the agent and the delivery agent are covalently linked. In some embodiments, the agent and the delivery agent are linked by a cleavable linker, such as a protease-sensitive linker, a pH-sensitive linker, or a glutathione-sensitive linker, examples of which are well known in the relevant art. Additional examples of cleavable linkers are provided in Donaghy, mAbs. 2016; 8(4):659-71, which is incorporated herein by reference. In some embodiments, the agent and the delivery agent are linked by a non-cleavable linker. In some embodiments, the delivery agent enhances delivery of the agent to intestinal epithelial cells of the subject, as compared to delivery of the agent to intestinal epithelial cells of the subject in the absence of the delivery agent. In some embodiments, the delivery agent enhances internalization (absorption) of the agent by intestinal epithelial cells of the subject, as compared to internalization (absorption) of the agent by intestinal epithelial cells of the subject in the absence of the delivery agent.

In some embodiments, a method described herein for treating a subject having an inflammatory disease present in intestinal epithelia (e.g., IBD) results in a reduction of inflammation in intestinal epithelial cells of the subject. In some embodiments, a method described herein results in a reduction of inflammation in intestinal epithelial cells of the subject by at least 10%, at least 20%, at least 30%, at least 40%, at least 50%, at least 60%, at least 70%, at least 80%, at least 90%, at least 95%, at least 99%, or 100%. A person of ordinary skill in the art is sufficiently capable of assessing inflammation in intestinal epithelial cells and may, for example, assess changes in the levels of inflammatory cytokines and/or chemokines present in intestinal epithelia of the subject.

In some embodiments, a method described herein for treating a subject having an inflammatory disease present in intestinal epithelia (e.g., IBD) results in a reduction of cell death in intestinal epithelial cells of the subject. In some embodiments, the cell death is cell death as a result of apoptosis, pyroptosis, and/or necroptosis. In some embodiments, a method described herein results in a reduction of cell death in intestinal epithelial cells of the subject by at least 10%, at least 20%, at least 30%, at least 40%, at least 50%, at least 60%, at least 70%, at least 80%, at least 90%, at least 95%, at least 99%, or 100%. A person of ordinary skill in the art is sufficiently capable of assessing cell death occurring in intestinal epithelial cells and may, for example, assess changes in the cellular levels of factors associated with apoptosis, pyroptosis, and/or necroptosis signaling pathways.

In some embodiments, a method described herein for treating a subject having an inflammatory disease present in intestinal epithelia (e.g., IBD) results in increased proliferation of intestinal epithelial cells of the subject. In some embodiments, a method described herein results in an increase in proliferation of intestinal epithelial cells of the subject by at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, or at least 10-fold or more. A person of ordinary skill in the art is sufficiently capable of assessing the level of proliferation occurring in intestinal epithelial cells and may, for example, assess changes in the cellular levels of factors associated with proliferation signaling pathways (e.g., mitosis), or by assessing changes in the overall level of DNA synthesis.

In some embodiments, a method described herein for treating a subject having an inflammatory disease present in intestinal epithelia (e.g., IBD) results in enhanced tissue repair in intestinal epithelial cells of the subject. In some embodiments, a method described herein results in an enhancement in tissue repair of intestinal epithelial cells of the subject by at least 10%, at least 20%, at least 30%, at least 40%, at least 50%, at least 60%, at least 70%, at least 80%, at least 90%, at least 2-fold, at least 3-fold, at least 4-fold, at least 5-fold, at least 6-fold, at least 7-fold, at least 8-fold, at least 9-fold, or at least 10-fold or more. A person of ordinary skill in the art is sufficiently capable of assessing the level of tissue repair occurring in intestinal epithelial cells and may, for example, assess changes in the cellular levels of factors associated with tissue repair.

A “subject” to which administration is contemplated herein may refer to a human (i.e., male or female of any age group, e.g., pediatric subject (e.g., infant, child, or adolescent) or adult subject (e.g., young adult, middle-aged adult, or senior adult)) or a non-human animal. In some embodiments, the subject is a human patient (e.g., a human patient known to have an IBD, e.g., Crohn's disease (CD) or ulcerative colitis (UC)). In some embodiments, the non-human animal is a mammal (e.g., rodent, e.g., mouse or rat), a primate (e.g., cynomolgus monkey or rhesus monkey), a commercially relevant mammal (e.g., cattle, pig, horse, sheep, goat, cat, or dog), or a bird (e.g., commercially relevant bird, such as chicken, duck, goose, or turkey). The non-human animal may be a male or female at any stage of development and may be a juvenile animal or an adult animal. The non-human animal may be a transgenic animal or genetically engineered animal.

In some embodiments, the subject is a companion animal (e.g., a pet or service animal). “A companion animal,” as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a research animal. Non-limiting examples of research animals include rodents (e.g., rats, mice, guinea pigs, and hamsters), rabbits, or non-human primates.

Conventional methods, known to those of ordinary skill in the art of medicine, can be used to administer an agent described herein or a composition thereof (e.g., a pharmaceutical composition) to the subject, depending upon the type of disease (e.g., IBD) to be treated or the site of the disease (e.g., intestinal epithelia). An agent or composition thereof (e.g., a pharmaceutical composition) can be administered systemically (i.e., throughout the body) or locally (i.e., to one or more specific organs, tissues, or locations in the body). The agent or composition thereof (e.g., a pharmaceutical composition) can be administered via any conventional route, e.g., administered orally (enterally), rectally, or parenterally, or via an implanted reservoir. The term “parenteral” as used herein includes intravenous, intramuscular, intraarticular, and intraarterial injection or infusion techniques. In some embodiments, the agent or composition thereof (e.g., a pharmaceutical composition) is administered orally. In some embodiments, the agent or composition thereof (e.g., a pharmaceutical composition) is administered rectally. In some embodiments, the agent or composition thereof (e.g., a pharmaceutical composition) is administered via intravenous injection or infusion. In addition, the agent or composition thereof (e.g., a pharmaceutical composition) can be administered to the subject via injectable depot routes of administration such as using 1-, 3-, or 6-month depot injectable or biodegradable materials and methods. In some embodiments, the agent or composition thereof (e.g., a pharmaceutical composition) is administered is administered to intestinal epithelial cells of the subject, such as, for example, intestinal epithelial cells that are affected by an IBD.

In some embodiments, the administration occurs more than once. In some embodiments, the administration occurs once per day, once per 2 days, once per 3 days, once per 4 days, once per 5 days, once per 6 days, once per week, once per 2 weeks, once per 3 weeks, once per month, once per 2 months, once per 3 months, once per 4 months, once per 6 months, once per 7 months, once per 8 months, once per 9 months, once per 10 months, once per 11 months, or once per year.

### EXAMPLES

### Example 1: Evaluation of Agents for Inhibiting Ifnl Signaling in Intestinal Epithelia

Previous studies have demonstrated how interferon lambda (IFNL) delays reconstitution of intestinal epithelia damaged by inflammation by driving cell death through the ZBP1/caspase-8/gasdermin C pathway. Activation of this pathway results in pyroptosis of intestinal epithelial cells, thereby delaying the repair of tissue damaged by inflammation. Thus, it is hypothesized that inhibitors of IFNL signaling, such as, for example monoclonal antibodies or small molecules that inhibit the activity of IFNL or IFNL receptor (IFNLR), or small molecules that inhibit downstream signaling by IFNL, such as, for example ZBP1, caspase-8, gasdermin C, and other factors that are functionally related to PANoptosis and activated as a result of IFNLR activation. Similarly, inhibition could be achieved by RNA interference (e.g., shRNA or siRNA) or gene editing (e.g., via CRISPR/Cas) to reduce the expression of one or more of these factors in intestinal epithelia.

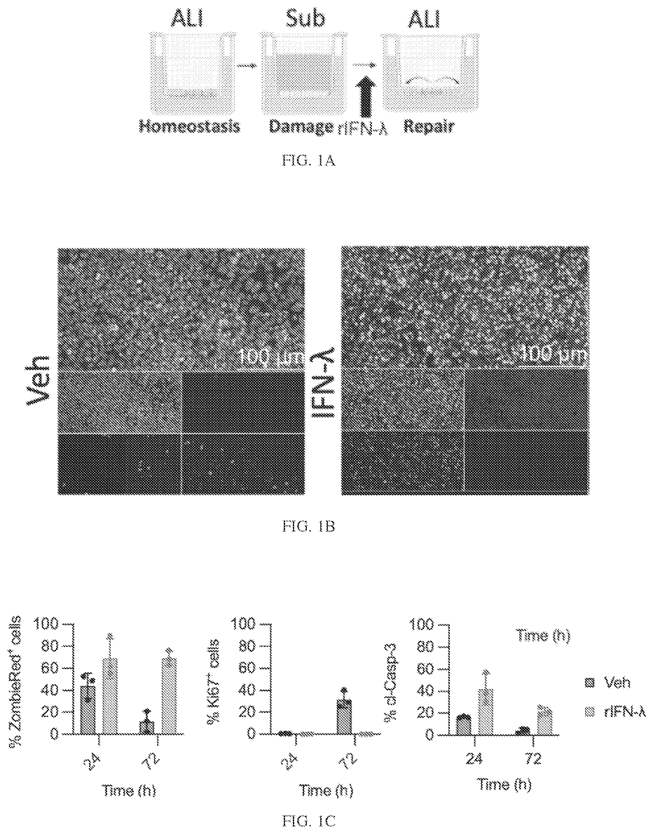

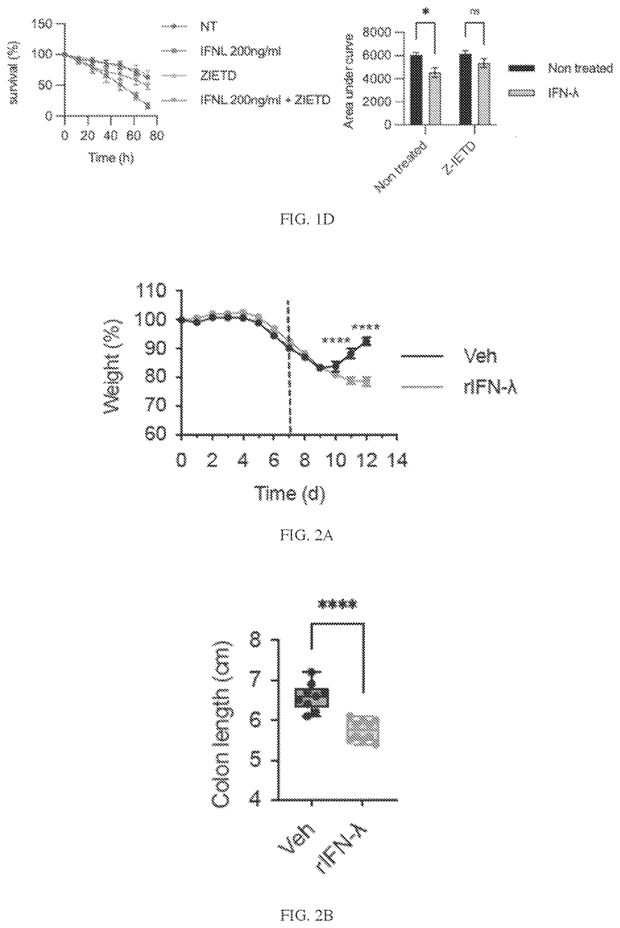

To test this hypothesis, inhibitory agents may be validated using an in vitro intestinal organoid model using previously established methods with murine intestinal epithelial organoids and an air-liquid interface (ALI) (FIG. 1A). Briefly, mouse small intestine organoids may be collected from freshly isolated intestinal crypts, seeded in transwells, and allowed to grow for 48 hours. Subsequent submersion of the air-exposed organoid monolayer mimics tissue damage. Organoids may be treated with recombinant IFNL prior to or following submersion in order to stimulate IFNL signaling in the monolayer. The ALI may then be reestablished, and the organoids optionally incubated with candidate agents for inhibiting IFNL signaling. After incubation with candidate agents, treated monolayers may then be assessed through a variety of techniques known in the art (FIGS. 1B and 1C). For example, cell survival may be assessed via fixable live/dead stain (i.e., Zombie dye), cell proliferation may be assessed by Ki67 staining, and IFNL signaling inhibition may be assessed by measuring the expression level or activity of factors involved in IFNL signaling (e.g., PANoptosome components). Alternately, this assay may also be performed with human organoids from a human subject (see Usui T, et al. “Preparation of Human Primary Colon Tissue-Derived Organoid Using Air Liquid Interface Culture.” Curr Protoc Toxicol. 2018; 75:22), such as a patient having an intestinal inflammatory disease, such as inflammatory bowel disease (IBD).

As a proof-of-principle, this assay was utilized to evaluate a candidate inhibitor of IFNL signaling, the small molecule Z-IETD-FMK which blocks the activity of caspase-8. Treatment with Z-IETD-FMK should therefore prevent activation of the ZBP1/caspase-8/gasdermin C pathway, even in the presence of recombinant IFNL. As expected, addition of 40 μm Z-IETD-FMK reduced the rate of cell death occurring due to pyroptosis in the organoid monolayer by approximately half (FIG. 1D). These data indicate that this in vitro model successfully recapitulates in vivo IFNL signaling and therefore provides a platform for comprehensive evaluation of candidate agents for IFNL signaling inhibition.

### Example 2: Type III Interferons Induce Pyroptosis in Gut Epithelial Cells and Delay Tissue Restitution Upon Acute Intestinal Injury

### Abstract

Tissue damage and repair are hallmarks of the inflammatory process. Despite a wealth of information focused on the mechanisms that govern tissue damage, mechanistic insight on how inflammatory immune mediators affect the restitution phase is lacking. Here, it was investigated how interferons influence tissue restitution after damage of the intestinal mucosa driven by inflammatory or physical injury. It is possible that type III, but not type I, interferons serve a central role in the restitution process. Type III interferons induce the upregulation of ZBP1, caspase activation, and cleavage of gasdermin C, and drive epithelial cell death by pyroptosis, thus delaying tissue restitution. It is also possible that this pathway is transcriptionally regulated in IBD patients. The results highlight a new molecular signaling cascade initiated by the immune system that affects the outcome of the immune response by delaying tissue repair and that may have important implications for human inflammatory disorders.

### INTRODUCTION

The immune system has evolved to protect the host from external or internal threats, as well as to maintain homeostasis of the organs and tissues. The strong interrelationship between these two functions of the immune system is best exemplified during the restitution phase that follows mucosal damage, occurring as a consequence of an immune response. The skin, the lungs, the gut, and other mucosae are constantly exposed to microbial and, or physical perturbations and harbor multiple immune and non-immune cells that sense the presence of hostile environmental or endogenous factors and mount a defensive response. The causative agent of this response, the response itself, or both, may lead to tissue damage. Tissue damage sensing by tissue-resident as well as newly recruited cells initiates a complex cascade of cellular and molecular processes to restore tissue functionality and homeostasis, or to adapt to persistent perturbations (Meizlish, Franklin et al. 2021).

The gastrointestinal tract represents an ideal tissue to explore the mechanisms underlying the exquisite balance between tissue damage and repair orchestrated by the immune system. In the intestine, immune cells, epithelial cells, and commensal microbes are in a dynamic equilibrium. A monolayer of highly specialized epithelial cells separates the gut lumen from the underlying lamina propria. The interplay between microbiota-derived inflammatory cues and the host cells in the intestine profoundly impacts the biology of the gut, both during homeostasis, inflammation, and damage responses. The lamina propria hosts a large variety of immune and non-immune cells that detect alterations in the functioning as well as in the integrity of the epithelial barrier and mount an immune response. The fine equilibrium between the microbiota, the epithelial barrier, and the immune system is lost during inflammatory bowel diseases (IBDs). IBDs are a group of heterogeneous diseases, whose pathogenesis is associated with genetic and environmental factors, that are characterized by a dysregulated immune response (Danese and Fiocchi 2011, Roda, Chien Ng et al. 2020). Along with a heightened inflammatory response, IBDs are characterized by the breach of the intestinal barrier and a defective repair response that compromises mucosal homeostasis. Therefore, the ability of immune mediators to influence epithelial repair has an important impact on the pathogenesis of IBDs. Indeed, the promotion of mucosal healing has been recognized as a major therapeutic challenge for the management of IBDs (Pineton de Chambrun, Peyrin-Biroulet et al. 2010).

It is possible for a group of interferons (IFNs), known as type III IFNs or IFN-λ (Kotenko, Gallagher et al. 2003, Sheppard, Kindsvogel et al. 2003, Prokunina-Olsson, Muchmore et al. 2013), to limit inflammation in a mouse model of colitis by dampening the tissue-damaging functions of neutrophils (Broggi, Tan et al. 2017). IFN-λ, as type I IFNs, plays potent anti-microbial roles, but, in contrast to type I IFNs, also preserves gut functionality by limiting excessive damage (Broggi, Granucci et al. 2020). The limited damage is largely explained by the fact that the expression of the IFN-λ receptor (IFNLR) is mainly restricted to epithelial cells and neutrophils. In contrast, type I IFNs act systemically and play potent inflammatory activities on immune and non-immune cells thanks to the broad expression of the type I IFN receptor (IFNAR). The local activity of IFN-λ at mucosal tissues, thus, limits the extent of activation of immune cells, preventing excessive tissue damage, while preserving the anti-microbial functions of IFN-λ (Broggi, Granucci et al. 2020).

Although it is possible for IFN-λ to limit intestinal tissue damage, the involvement of this group of IFNs during tissue restitution of the gut is more controversial. Indeed, IFN-λ and type I IFNs may function in a balanced and compartmentalized way to favor re-epithelization by acting, respectively, on epithelial cells or immune cells resident in the lamina propria (McElrath, Espinosa et al. 2021). IFN-λ has been proposed to facilitate the proliferation of intestinal epithelial cells via STAT1 signaling (Chiriac, Buchen et al. 2017) and to partially enhance gut mucosal integrity during graft versus host disease (Henden, Koyama et al. 2021). On the other hand, IFN-λ and, or the IFNLR were found to be upregulated in IBD patients (Chiriac, Buchen et al. 2017, Gunther, Ruder et al. 2019). Further, systemic and prolonged overexpression of IFN-λ in mice favored the death of Paneth cells, a group of cells that can facilitate epithelial cell regeneration by acquiring stem-like features (Schmitt, Schewe et al. 2018), and by regulating the balance of epithelial growth factors in the stem cell niche (Sato, van Es et al. 2011).

In keeping with a possible detrimental role of IFN-λ during an inflammatory response at mucosal surfaces, it is possible that IFN-λ delays the proliferation of lung epithelial cells in murine models of persistent viral infections (Broggi, Ghosh et al. 2020, Major, Crotta et al. 2020). In addition, IFN-λ production in the lower respiratory tract of COVID-19 patients is associated with increased apoptotic and decreased proliferative transcriptional programs, and characterizes SARS-CoV-2-infected individuals with severe-to-critical outcomes (Sposito, Broggi et al. 2021). Whether IFN-λ plays similar roles in the intestine, and the molecular mechanisms initiated by this group of IFNs to exert their functions during gut restitution, remain unknown.

By exploiting conditional knock-out mice that do not respond to IFN-λ only in intestinal epithelial cells or in neutrophils, ex vivo transcriptomics, and biochemical assays, as well as intestinal organoids in vitro, the role of IFN-λ during tissue repair secondary to either an inflammatory insult or to radiation damage was evaluated further. The data reveal a new molecular cascade initiated by IFN-λ that culminates in the activation of ZBP1 and of gasdermin C (GSDMC), in the induction of pyroptosis and results in delayed gut restitution.

### Results.

### IFN-λ Delayed Tissue Repair of the Inflamed Gut.

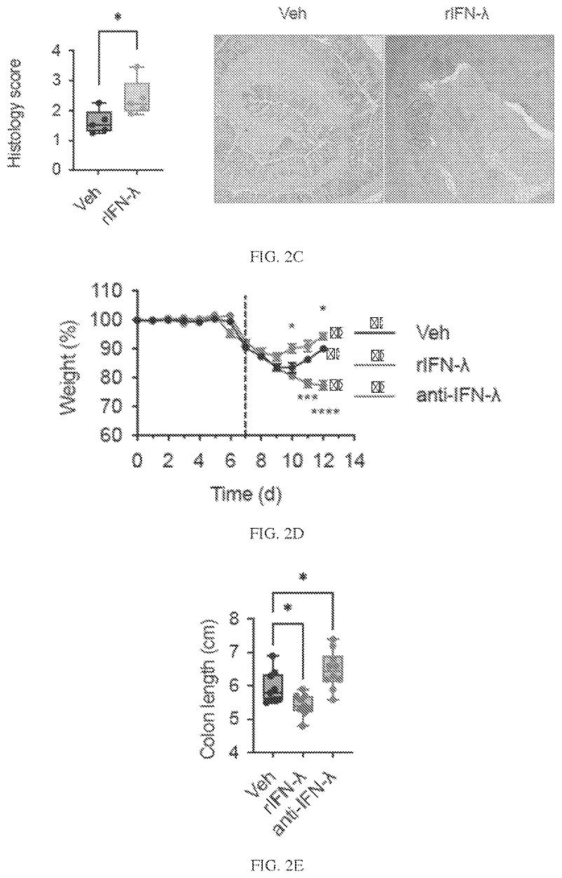

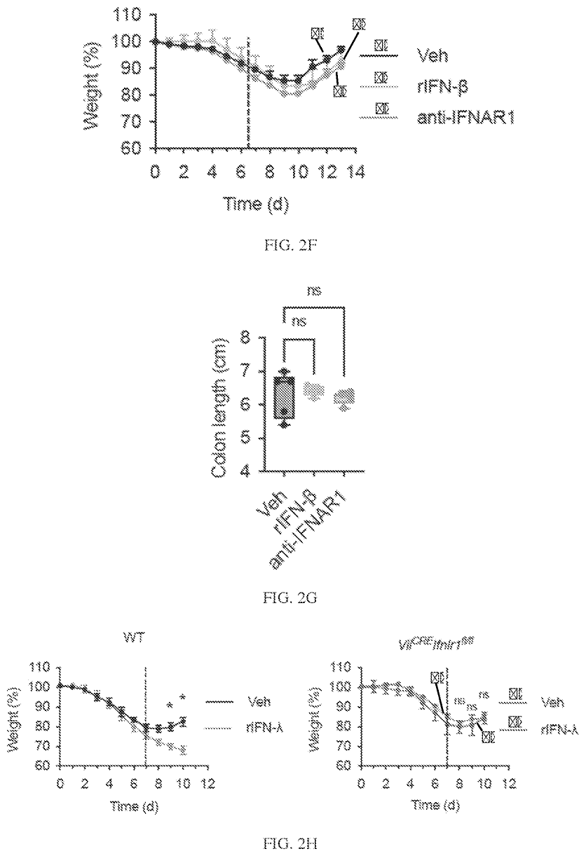

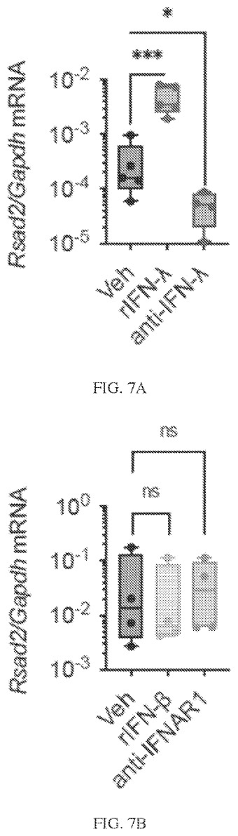

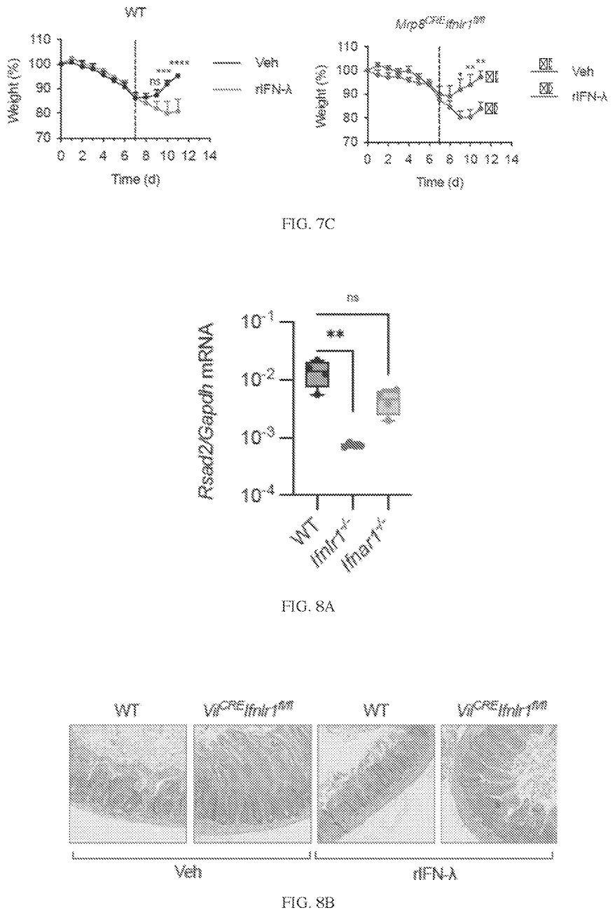

It is possible that in the acute inflammatory phase of the dextran sulfate sodium (DSS) model of colitis, IFN-λ signaling in neutrophils dampens reactive oxygen species production and neutrophil degranulation, and thus restrains intestinal damage (Broggi, Tan et al. 2017). To assess the involvement of IFN-λ during the restitution phase of the DSS colitis model, recombinant (r)IFN-λ was, and in some cases was not, injected in mice after DSS-induced inflammation peaked. Therefore, it is possible that rIFN-λ administration upregulated interferon-stimulated genes (ISGs) in the colon of DSS-treated mice (FIG. 7A). Mice administered rIFN-λ, but not vehicle controls, showed persistent weight loss, reduced colon length, and prolonged tissue damage as measured by histology (FIGS. 2A-2C). These data suggest that IFN-λ delays tissue restitution in mice encountering colitis.

Whether the endogenous IFN-λ, which is produced during colitis development (Broggi, Tan et al. 2017), also affects the restitution phase was tested. After the peak of the inflammatory process induced by DSS administration, mice were treated with a blocking antibody directed against IFN-λ and compared to mice treated with DSS, in the presence or absence of rIFN-λ. The data demonstrated that inhibition of endogenous IFN-λ facilitates tissue restitution as measured by increased weight gain and colon lengthening (FIGS. 2D-2E). The data indicated that ISG levels in epithelial cells were significantly decreased in mice that were treated with the anti-IFN-λ antibody (FIG. 7A), suggesting that IFN-λ, rather than type I IFNs, plays a major role in driving gene transcription during the repair phase of colitis. To directly test the involvement of type I IFNs in the restitution phase of DSS-induced colitis, either type I IFN signaling was blocked by using an anti-IFNAR antibody, or rIFNβ was added, 7 days after DSS administration. In keeping with a possible role of IFN-λ in regulating mucosal epithelial responses, none of the treatments aimed at targeting type I IFNs affected tissue repair (FIGS. 2F-2G). Accordingly, ISG levels in colonocytes were not altered under these experimental conditions when compared to control mice (FIG. 7B).

While intestinal epithelial cells are the major effector cell type during mucosal restitution, other cells, including immune cells, can participate in modulating tissue repair. Since intestinal epithelial cells and neutrophils are the two cell types that respond to IFN-λ in the gut of mice (Broggi, Tan et al. 2017), conditional knock out mice that do not express the IFNLR either in intestinal epithelial cells (Vil^{CRE}Ifnlr1^{fl/fl} mice) or neutrophils (Mrp8^{CRE}Ifnlr1^{fl/fl} mice) were used. Ifnlr1^{fl/fl} (WT) littermates were used as controls. In contrast to WT littermates, administration of rIFN-λ to Vil^{CRE}Ifnlr1^{fl/fl} mice did not delay tissue restitution as measured by weight change (FIG. 2H). Vil^{CRE}Ifnlr1^{fl/fl} mice in which intestinal epithelial cells do not respond to IFN-λ showed a faster recovery as measured by a significant increase in colon length, regardless of the presence or absence of rIFN-λ (FIG. 2I). In contrast to Vil^{CRE}Ifnlr1^{fl/fl} mice, Mrp8^{CRE}Ifnlr1^{fl/fl} behaved similarly to their WT counterpart, in the presence or absence of rIFN-λ (FIG. 7C). These data demonstrate that, in contrast to the acute inflammatory phase of colitis, epithelial cells, not neutrophils, are the major responders to endogenous, as well as exogenous, IFN-λ and that IFN-λ signaling in epithelial cells delays tissue restitution.

IFN-λ Delayed the Tissue Restitution Phase that Follows Radiation Damage.

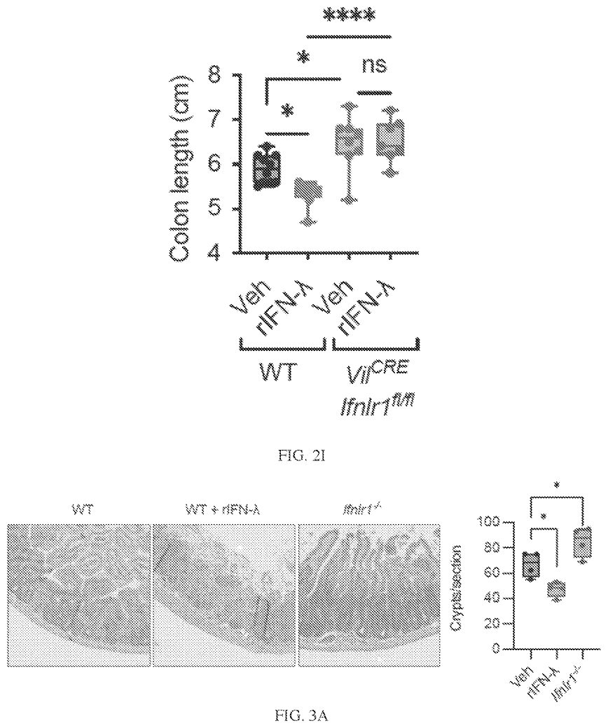

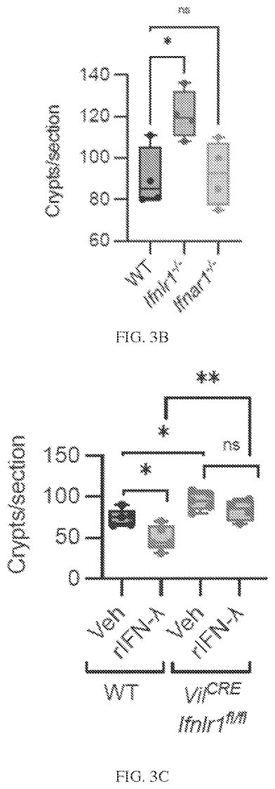

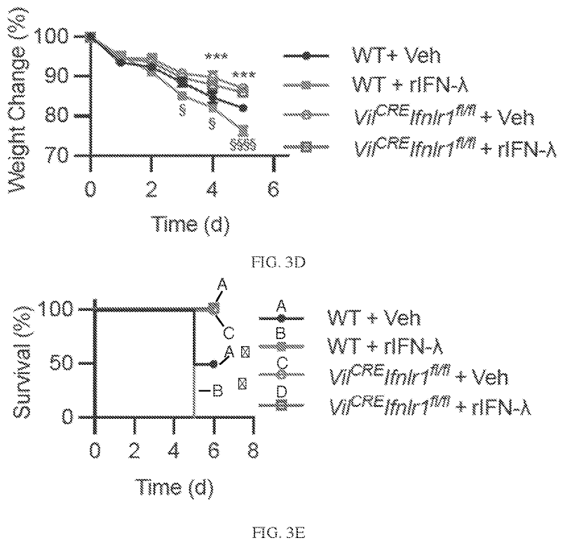

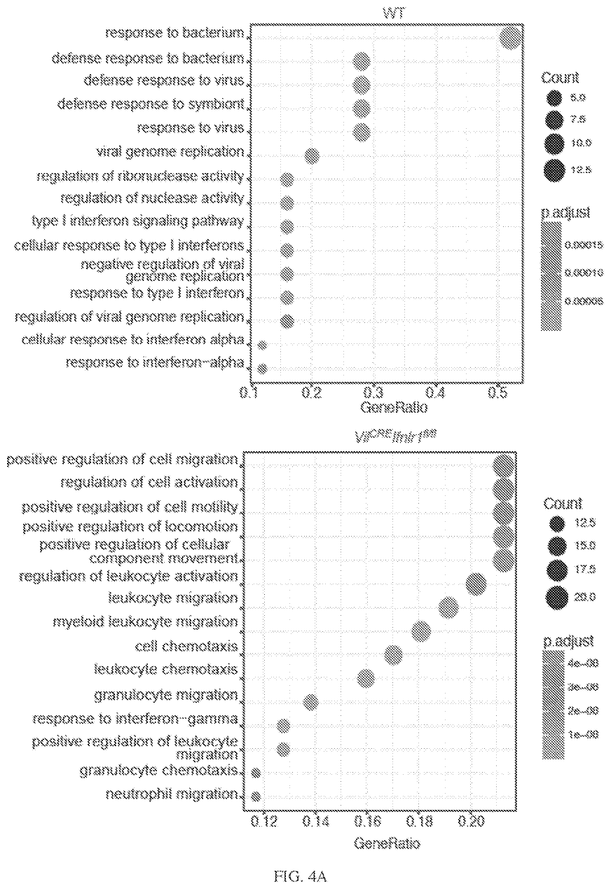

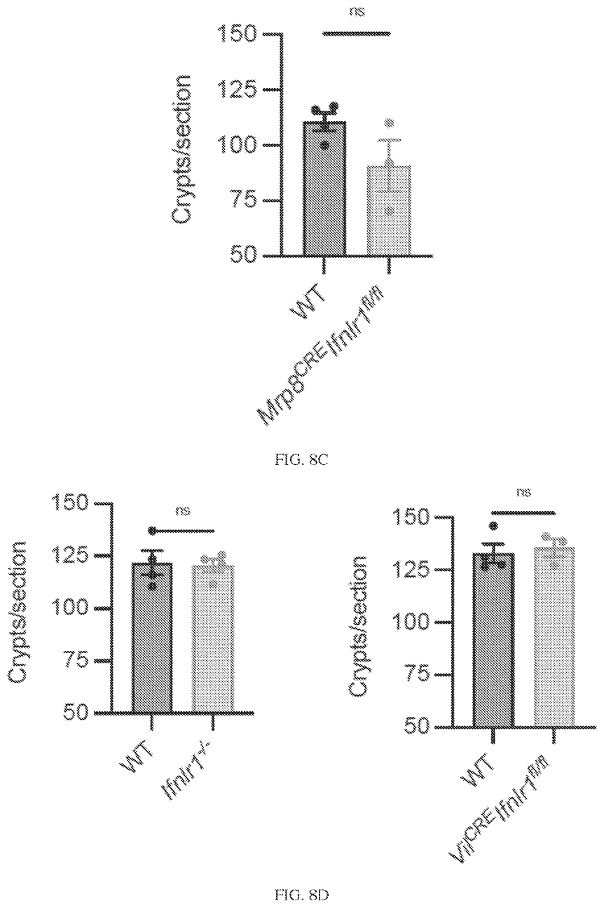

Repair of the gut epithelial monolayer is a complex process, and the regenerative capacity of intestinal stem cells (ISCs) plays a critical role (Blanpain and Fuchs 2014). To target ISCs and assess the direct involvement of IFN-λ during gut restitution, a well-characterized model of epithelial damage resulting from exposure to ionizing radiations was employed (Kim, Yang et al. 2017). In this model, radiation induces widespread epithelial cell death in the small intestine, with a particularly dramatic effect on cycling ISCs that reside at the bottom of the small intestinal crypt. Cell death is followed by repair of the damaged epithelial crypts and return to homeostasis. Three to four days after radiation injury, during the peak of the repair response, crypt regeneration was assessed in WT mice, WT mice administered exogenous rIFN-λ, or Ifnlr1^{-/-} mice. It was determined that mice that received rIFN-λ showed reduced regeneration of the crypts, while Ifnlr1^{-/-} mice had an increased number of crypts per histological section (crypts/section) (FIG. 3A). Notably, in the small intestine of irradiated mice, similarly to what was observed in the colon of mice exposed to DSS, endogenous IFN-λ, but not type I IFN, signaling caused the delay in tissue restitution (FIG. 3B). Similarly, ISG induction in epithelial cells was dependent on IFN-λ, rather than type I IFNs (FIG. 8A).

Next, the nature of the cell types that respond to IFN-λ in the irradiated small intestine was assessed. When Vil^{CRE}Ifnlr1^{fl/fl} mice and WT littermates were used, it was determined that the number of crypts three days post-radiation was significantly increased in Vil^{CRE}Ifnlr1^{fl/fl} mice compared to WT mice (FIGS. 3C and 8B). It was also demonstrated that exogenous rIFN-λ does not affect the number of crypts/section in Vil^{CRE}Ifnlr1^{fl/fl} mice, while delaying tissue restitution in WT littermates (FIGS. 3C and 8B). In keeping with a possible role for epithelial cells, but not neutrophils, in responding to IFN-λ during tissue restitution, it was determined that Mrp8^{CRE}Ifnlr1^{fl/fl} mice didn't show significant differences compared to their WT littermates (FIG. 8C). No differences were measured in the number of crypts/section of non-irradiated mice regardless of their capacity to respond, or not, to IFN-λ (FIG. 8D). Irradiated Vil^{CRE}Ifnlr1^{fl/fl} mice or WT littermates, treated or not with rIFN-λ, were followed over time. It was found that WT mice irradiated and treated with rIFN-λ lost significantly more weight than irradiated WT mice, and all died (FIGS. 3D-3E). Notably, WT littermates lost significantly more weight compared to Vil^{CRE}Ifnlr1^{fl/fl} mice, treated or not with rIFN-λ (FIG. 3D). In contrast, Vil^{CRE}Ifnlr1^{fl/fl} mice treated or not with rIFN-λ showed a very similar behavior (FIGS. 3D-3E). Overall, these data demonstrate that epithelial cell regeneration and tissue restitution in the small intestine of irradiated mice is inhibited in the presence of IFN-λ. Also, that IFN-λ delays repair by acting on intestinal epithelial cells.

### IFN-λ Dampened Regenerative and Proliferative Transcriptional Programs in Intestinal Epithelial Cells.

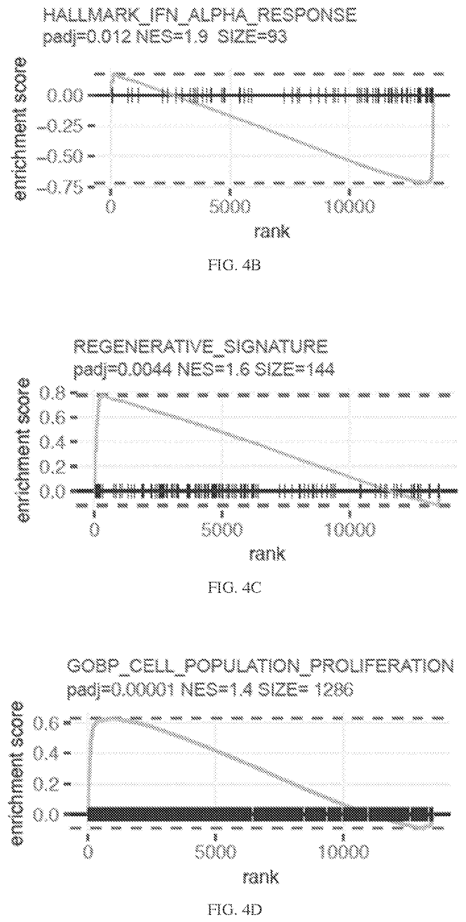

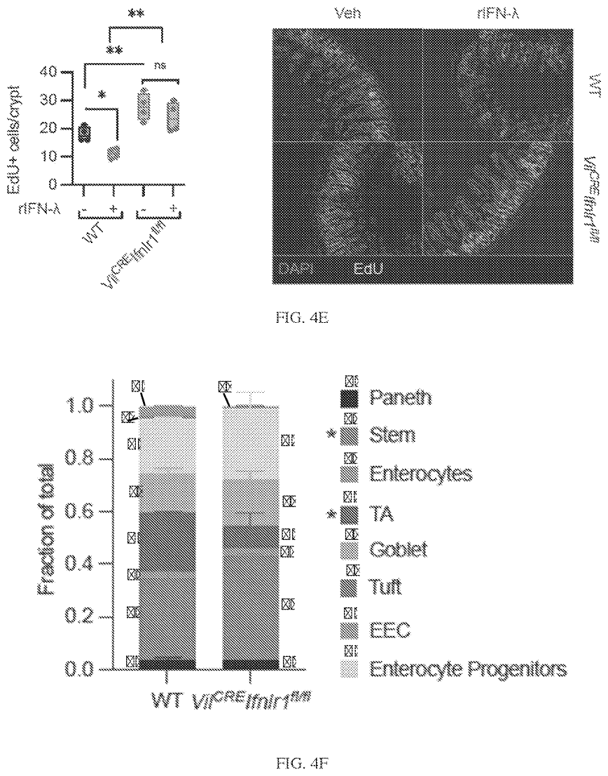

To determine the transcriptional programs initiated by IFN-λ to delay tissue restitution, intestinal crypts from the small intestine of Vil^{CRE}Ifnlr1^{fl/fl} mice or WT littermates that have been irradiated were isolated and targeted transcriptomics analysis (RNAseq) was performed. In keeping with a major role of IFN-λ-dependent responses in the intestine, when gene ontology (GO) enrichment analyses were performed, IFN-signaling related pathways, as well as anti-viral or anti-bacterial pathways, were highly enriched in WT epithelial cells, compared to Vil^{CRE}Ifnlr1^{fl/fl} (FIG. 4A). In contrast, GO terms associated with cell migration and extracellular remodeling, which are linked to higher efficiency in the closure of mucosal wounds (Quirós and Nusrat 2018), were mostly represented in epithelial cells that do not respond to IFN-λ (FIG. 4A).

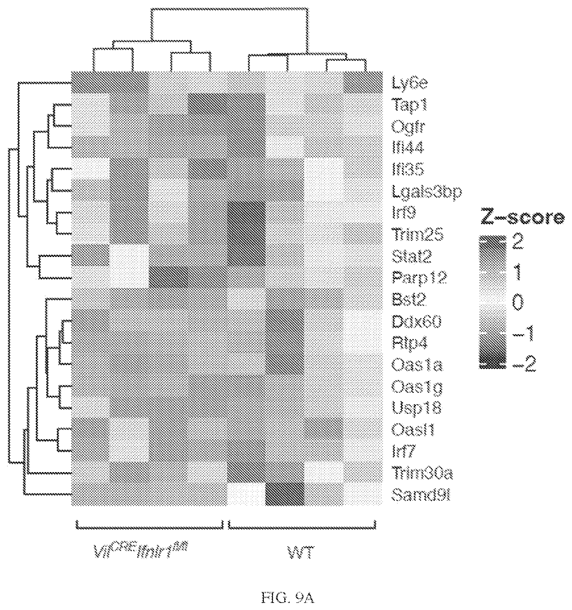

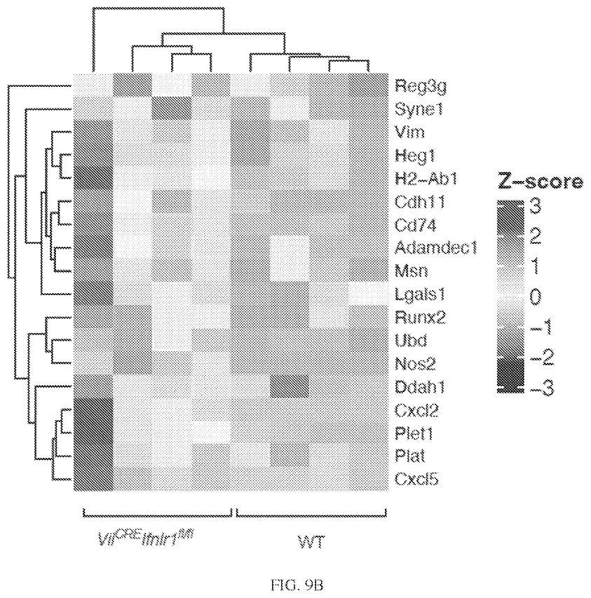

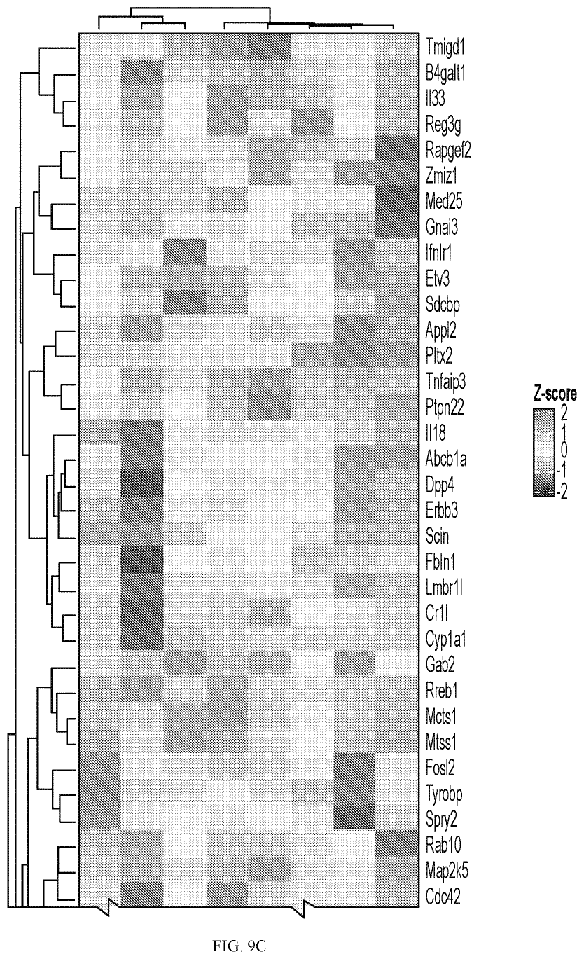

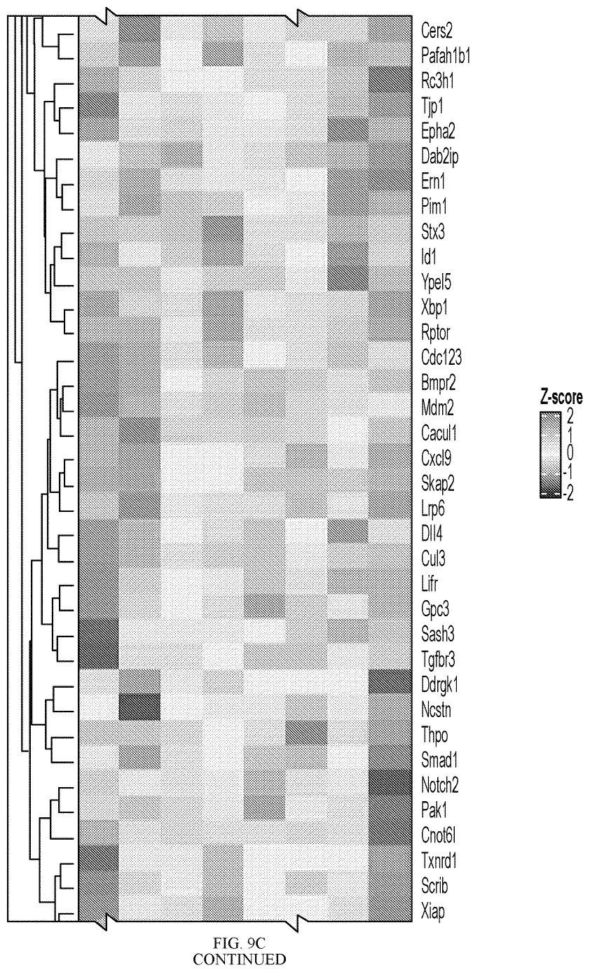

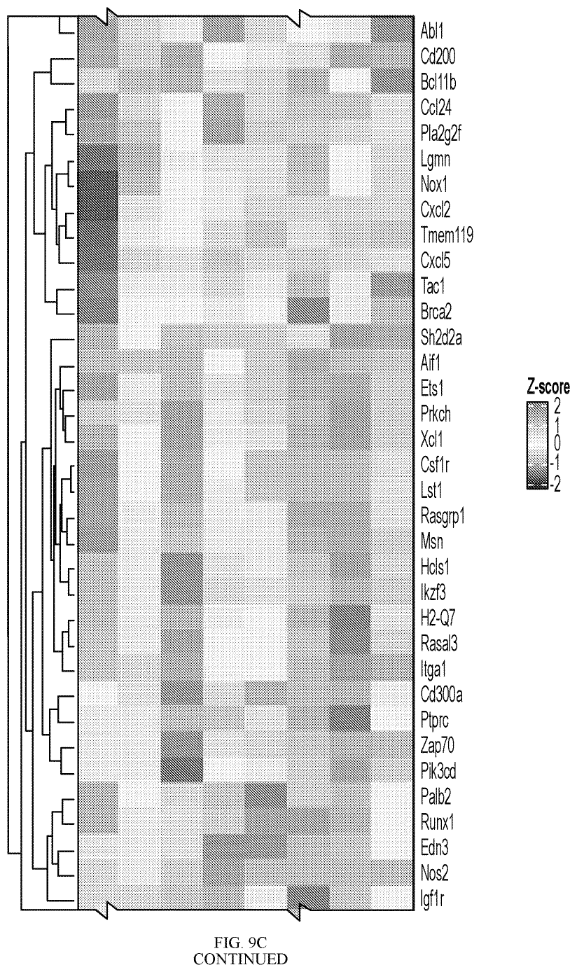

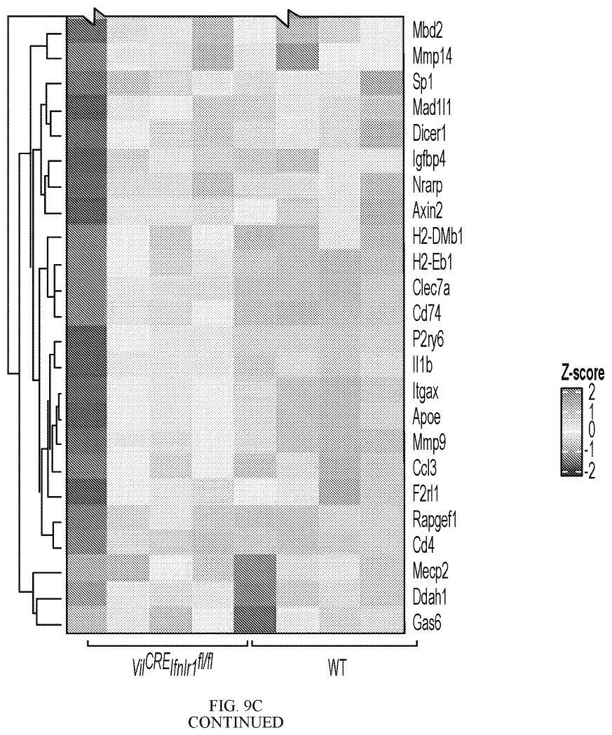

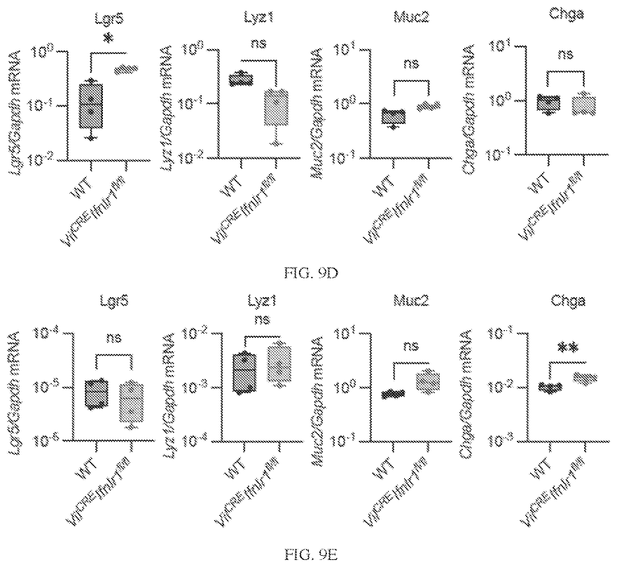

Gene set enrichment analysis (GSEA) confirmed that genes associated with IFN responses were significantly enriched in WT, compared to knock-out, epithelial cells (FIGS. 4A and 9A). The relative enrichment of a previously identified colitis-associated regenerative epithelial gene-set was assessed (Yui, Azzolin et al. 2018), as well as the gene-sets associated with epithelial cell proliferation. Both gene-sets were significantly enriched when epithelial cells did not respond to IFN-λ (FIGS. 4C-4D and 9B).

To assess whether IFN-λ-dependent delayed tissue restitution is characterized by reduced cell proliferation in vivo, the thymidine analog 2′-deoxy-5-ethynyluridine (EdU) was administered two hours before mice were euthanized and measured cell proliferation in either WT littermates or Vil^{CRE}Ifnlr1^{fl/fl} mice, administered or not rIFN-λ. It was determined that exogenous rIFN-λ reduced the number of EdU-positive cells per crypt in WT but not Vil^{CRE}Ifnlr1^{fl/fl} mice (FIG. 4E), and that Vil^{CRE}Ifnlr1^{fl/fl} mice had a significant increased number of proliferating cells per crypts, compared to WT littermates, irrespectively of the administration of rIFN-λ (FIG. 4E).

During tissue repair that follows radiation damage (Metcalfe, Kljavin et al. 2014) or colitis (VanDussen, Sonnek et al. 2019), specialized ISCs drive re-epithelialization by massively proliferating. Therefore, the decreased number of proliferating epithelial cells in WT mice may reflect the lack of reparatory ISCs that proliferate. To assess whether endogenous IFN-λ affected the cellular composition of the small intestine in WT or Vil^{CRE}Ifnlr1^{fl/fl} mice that were irradiated, CIBERSORTx was used (Newman, Steen et al. 2019) and the bulk RNAseq data was deconvoluted based on single-cell RNAseq data previously published (Haber, Biton et al. 2017). The deconvolution analysis revealed that, while most epithelial cell types did not present major significant differences, the ISC compartment was significantly expanded in mice that were irradiated and whose epithelial cells do not respond to IFN-λ (FIG. 4F). In keeping with retro-differentiation of transit-amplifying (TA) cells to replenish the ISC compartment upon ISC depletion (Wang, Chiang et al. 2019, Ohara, Colonna et al. 2022), TA cells were significantly decreased in the small intestine of Vil^{CRE}Ifnlr1^{fl/fl} mice, compared to WT controls (FIG. 4F). The expansion of the Lrg5⁺ compartment in mice that do not respond to IFN-λ was confirmed by qPCR (FIG. 9D). The data also confirmed that the major epithelial cell populations analyzed were not different under homeostatic conditions in Vil^{CRE}Ifnlr1^{fl/fl} mice or WT littermates (FIG. 9E). Overall, these data demonstrate that IFN-λ initiates a transcriptional program that reduces tissues restitution, limits ISC cell expansion, and, thus, dampens the overall capacity of epithelial cells to proliferate.

### IFN-λ Controlled the Expression of ZBP1 and the Activation of Gasdermin C.

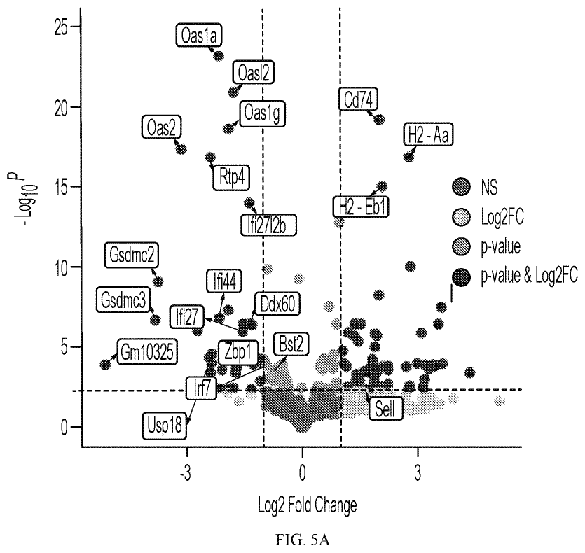

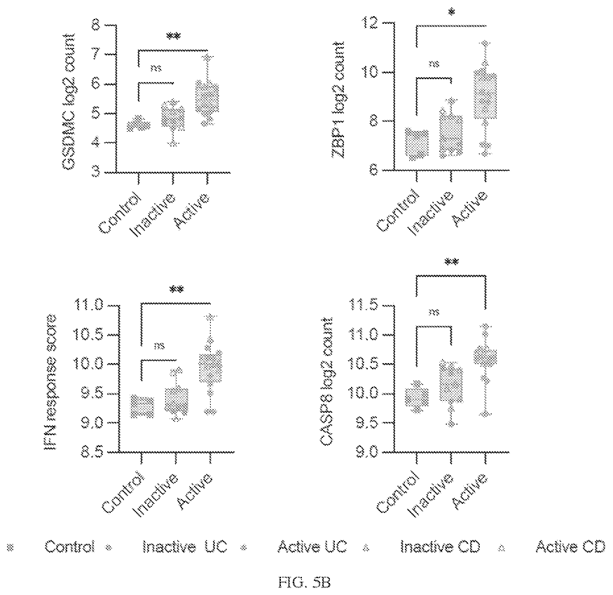

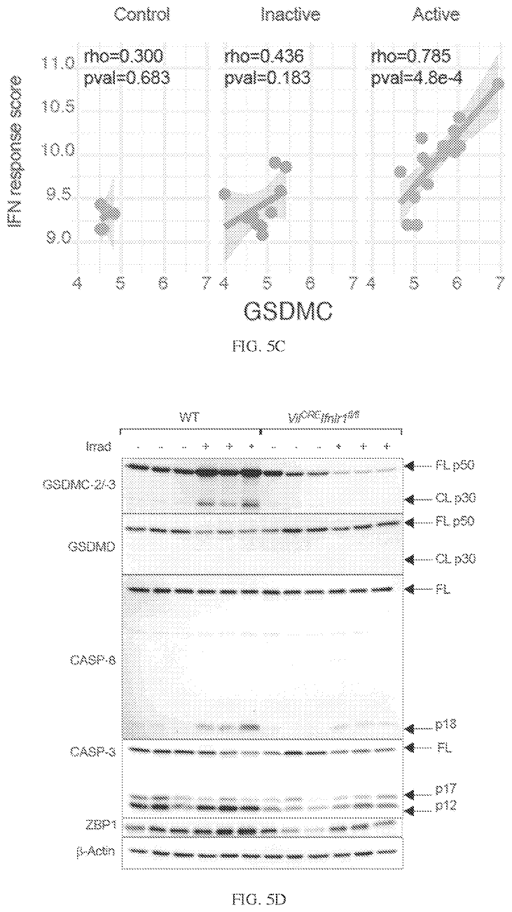

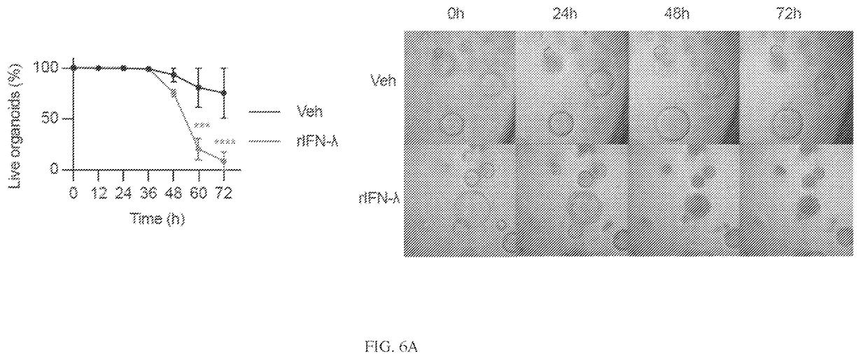

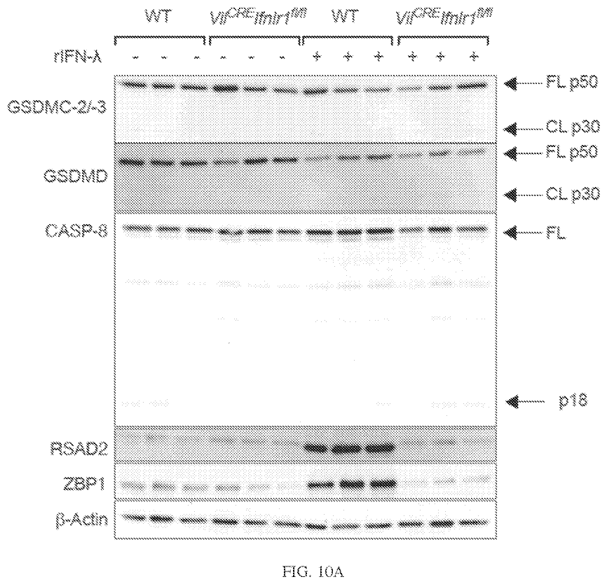

The reduced expansion of ISC can be driven either by increased cell death of ISCs and, or TA cells, reduced proliferative programs, or both. To determine the molecular mechanisms regulated by IFN-λ to dampen tissue restitution, the genes that were significantly differentially regulated in epithelial cells derived from irradiated Vil^{CRE}Ifnlr1^{fl/fl} mice were identified and compared to WT mice (FIG. 5A). As expected, multiple ISGs were among the genes significantly downregulated in cells that cannot respond to IFN-λ (FIG. 5A). Zbp1 was among these genes. ZBP1 is a possible component in the multiprotein complex PANoptosome, which encompasses effectors of several forms of cell death, and is an important regulator of cell fate (Kuriakose and Kanneganti 2018). It was found that protein levels of ZBP1, as well as another ISG such as RSAD2, were upregulated in epithelial cells of the small intestine upon in vivo administration of rIFN-λ in non-irradiated WT mice (FIG. 10A). Upregulation of these proteins was prevented in epithelial cells derived from Vil^{CRE}Ifnlr1^{fl/fl} mice and was not different in the absence of rIFN-λ in the two backgrounds (FIG. 10A). Among other genes significantly downregulated in cells that do not respond to IFN-λ, there were two members of the gasdermin C (GSDMC) family. GSDMs are critical effectors of pyroptosis, a form of inflammatory cell death (Kovacs and Miao 2017). Compared to other GSDMs, very little is known about the functions of GSDMC, and scattered reports have involved GSDMC in the lytic death of tumor cells (Hou, Zhao et al. 2020, Zhang, Zhou et al. 2021), or of enterocytes during helminth infections (Xi, Montague et al. 2021). Of note, non-irradiated mice administered with rIFN-λ do not show upregulation of the GSDMC protein (FIG. 10A), demonstrating that additional pathways associated with irradiation and, or tissue damage and repair regulate Gsdmc gene expression and, or protein synthesis.

IBD patients presented increased levels of IFN-λ and, or Ifnlr1 (Chiriac, Buchen et al. 2017, Gunther, Ruder et al. 2019). Prompted by the findings in irradiated mice, the expression levels of ZBP1 and GSDMC (the only GSDMC present in humans) were assessed in the biopsy derived from a cohort of IBD patients with active or inactive disease, or non-IBD controls (see Material and Methods for further details). A significant increase in the expression of ZBP1 as well as GSDMC in patients with active IBD, compared to non-IBD controls was observed (FIG. 5B). Similar expression trends were observed in RNAseq datasets derived from rectal mucosal biopsies from ulcerative colitis (UC) pediatric patients (PROTECT cohort) and from ileal biopsies from Crohn's disease (CD) pediatric patients (RISK cohort) in two independent cohorts previously published (Haberman, Tickle et al. 2014, Haberman, Karns et al. 2019) (FIG. 10B). In keeping with a possible role of IFNs in IBD patients, it was determined that genes regulated in response of IFNs (evaluated as mean expression of genes that belong to the “HALLMARK_IFN_ALPHA_RESPONSE” gene-set (Liberzon, Birger et al. 2015) and indicated as “IFN response score”) were significantly enriched in IBD patients with active disease, compared to controls or patients with inactive disease (FIG. 5B). In support of a role of IFN-λ in modulating GSDMC expression, GSDMC levels positively correlated with the IFN response score in patients with active disease, but not in the other subjects analyzed (FIG. 5C). These data indicate that IFN induction and upregulation of the genes that encode for ZBP1 and GSDMC are hallmarks of intestinal damage both in mice and humans.

GSDMs exert their pyroptotic function upon cleavage by caspases (Kovacs and Miao 2017), when the N-terminal cleavage product oligomerizes to form lytic pores in the cell membrane, leading to the loss of ionic homeostasis and cell death (Broz, Pelegrin et al. 2020). Whether GSDMC-2/-3 were cleaved in the epithelial cells of the small intestine upon irradiation was tested and confirmed that irradiated, but not non-irradiated, mice not only showed increased levels of GSDMC-2/-3, but also that GSDMC-2/-3 were efficiently cleaved in WT, but not Vil^{CRE}Ifnlr1^{fl/fl}, mice (FIG. 5D). In contrast, another possible effector of pyroptosis, GSDMD, was not activated.

GSDMC is primarily cleaved by Caspase-8 (Hou, Zhao et al. 2020, Zhang, Zhou et al. 2021). Indeed, the pattern of bands of cleaved GSDMC-2/-3 is compatible with the activity of Caspase-8 (Julien and Wells 2017). The activation of Caspase-8 was investigated. Caspase-8 was not activated in WT or Vil^{CRE}Ifnlr1^{fl/fl} mice in the absence of irradiation (FIG. 10A). In contrast, epithelial cells derived from irradiated WT littermates, but not Vil^{CRE}Ifnlr1^{fl/fl} mice, efficiently activated Caspase-8 (FIG. 5D). Of note, it was determined that expression of CASP8 was increased in patients with active IBD compared to non-IBD controls (FIGS. 5B and 10B). In keeping with the capacity of ZBP1 to control the activation of multiple caspases (Kuriakose and Kanneganti 2018), it was determined that a similar pattern of activation was also true for Caspase-3 (FIG. 5D).

Overall, these data demonstrate that IFN-λ initiates in the small intestine of irradiated mice a signaling cascade that allows the upregulation of ZBP1, the activation of Caspase-8/-3, and the induction and cleavage of GSDMC, an executor of pyroptosis. Also, that similar programs are transcriptionally upregulated in IBD patients with active disease.

### The ZBP1-GSDMC Axis Induced by IFN-λ Controlled Epithelial Cell Death and Proliferation.

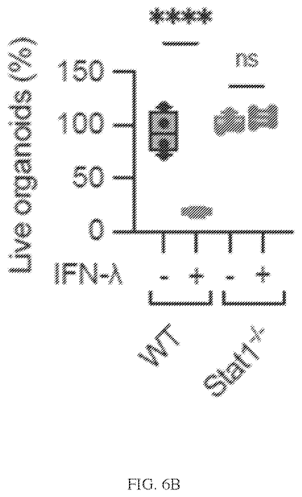

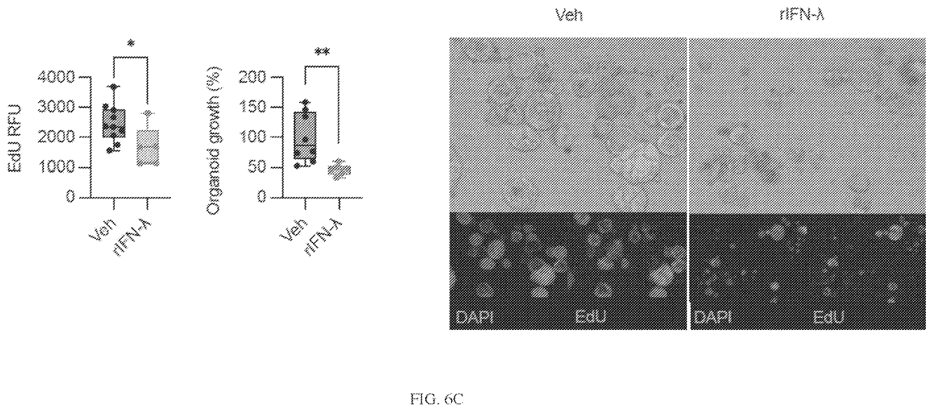

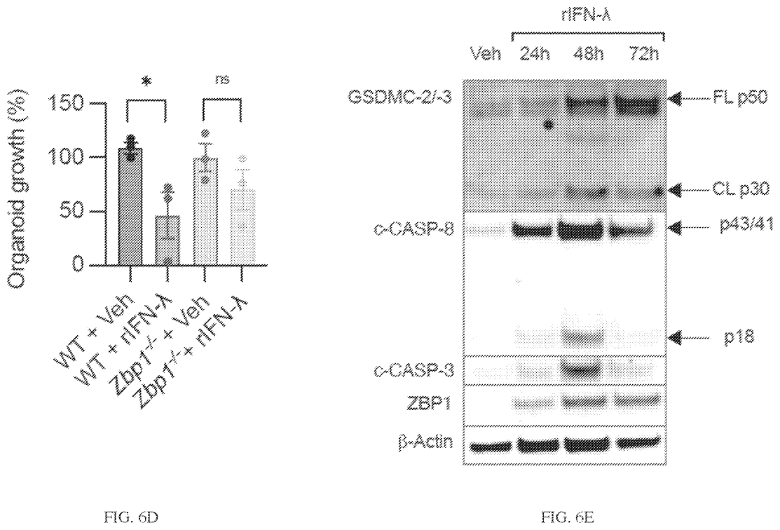

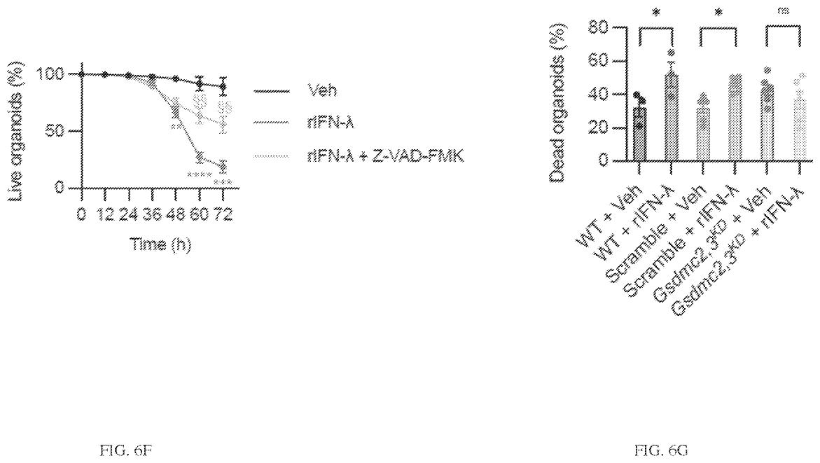

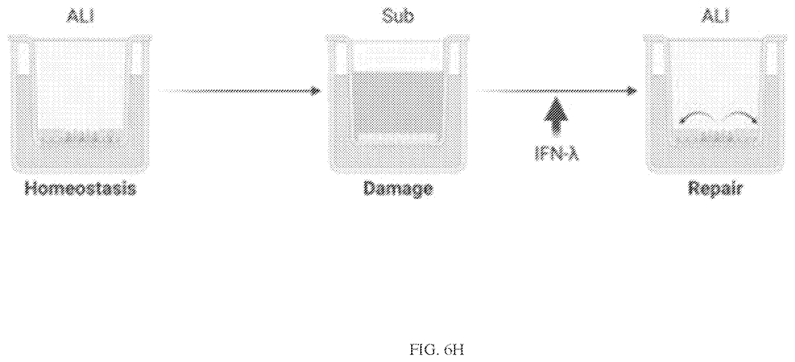

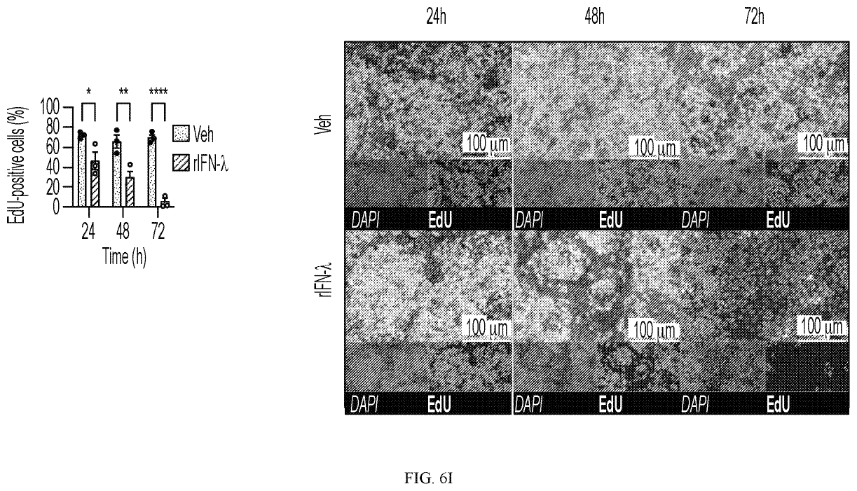

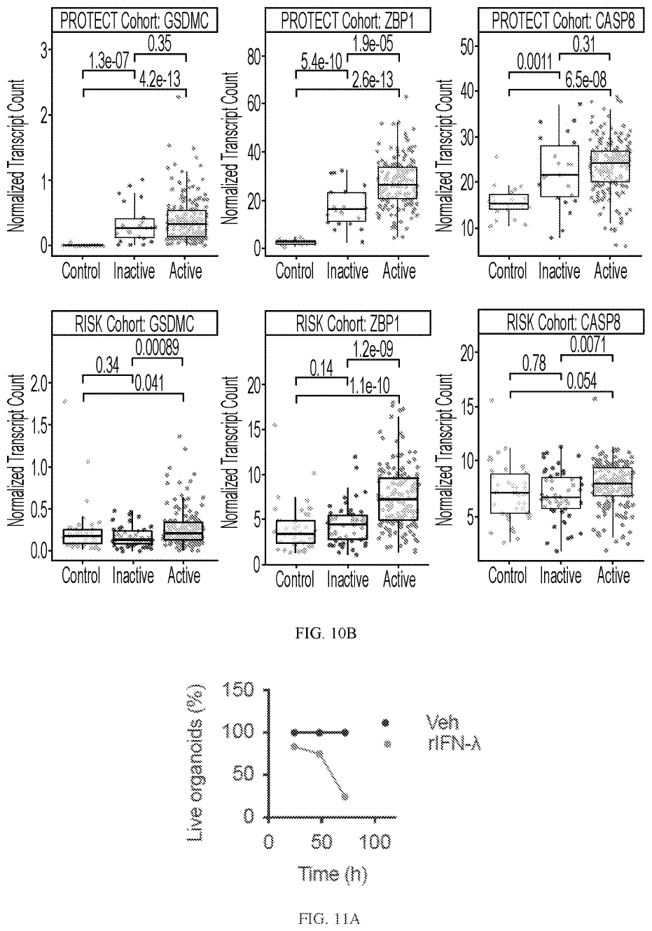

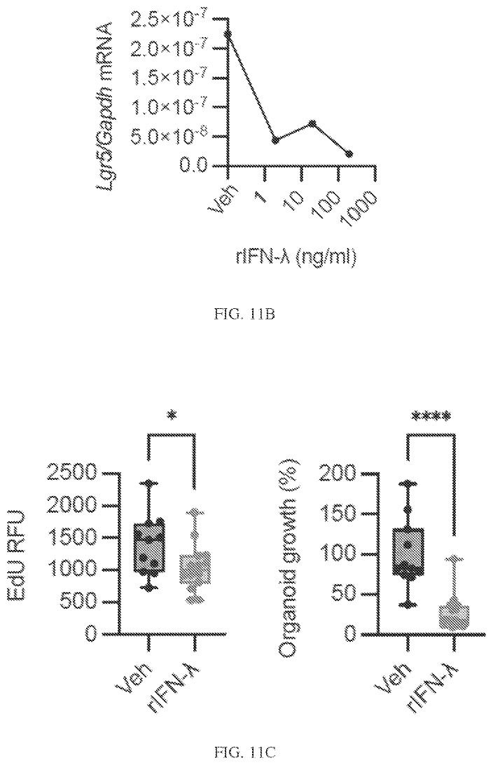

Intestinal organoids were used to directly assess the role of the signaling cascade initiated by IFN-λ in driving cell death. Mouse and human small intestinal organoids seeded in the presence of rIFN-λ died between 48 and 72 hours from treatment (FIGS. 6A and 11A).

Dying cells assumed typical changes associated with pyroptosis including swelling and sudden disruption of the plasma membrane and liberation of nuclear DNA (FIG. 6A). By using organoids derived from WT or Stat1^{-/-} mice, the notion that gene transcription induced by IFN-λ was necessary to induce cell death was confirmed (FIG. 6B). No differences were observed between the two genotypes in the absence of rIFN-λ (FIG. 6B). Similar to what was observed in irradiated epithelial cells in vivo, it was determined that rIFN-λ administration to small intestine organoids profoundly diminished the level of Lgr5 expression, suggesting a defect in the maintenance and, or proliferation of ISCs (FIG. 11B). In agreement with a reduced number of ISCs that proliferate, it was also determined that cell proliferation (as measured by EdU incorporation) was significantly decreased in IFN-λ-treated mouse, as well as human organoids (FIGS. 6C and 11C).

To assess the involvement of ZBP1 in these processes, organoids from either WT or Zbp1^{-/-} mice were derived. Organoids were differentiated in the presence or absence of IFN-λ and their survival and, or growth was followed for 72 h. Survival and growth of organoids, derived either from the small or large intestine, differentiated from WT, but not Zbp1^{-/-}, and mice were significantly reduced upon the administration of rIFN-λ (FIGS. 6D and 11D). In agreement with the capacity of IFN-λ to activate a ZBP1/Caspase-8/GSDMC axis, organoids grown for 6 days and then administered with rIFN-λ showed ZBP1 upregulation and cleavage of GSDMC-2/-3 and Caspase-8 and Caspase-3 (FIG. 6E). Furthermore, inhibition of caspase activity with the pan-caspase inhibitor Z-VAD-FMK protected the organoids from cell death induced by IFN-λ (FIG. 6F). To directly assess the involvement of GSDMC in this process, Gsdmc-2 and Gsdmc-3 were knocked down in small intestine organoids and it was determined that, similar to Zbp1^{-/-} organoids, upon exposure to rIFN-λ, survival of organoids that do not express Gsdmc2, 3 was significantly increased compared to controls (FIG. 6G).

To better reflect the cycles of injury and repair characteristic of IBD and mouse models of colitis, a previously described model of long-term organoid culture was implemented (Wang, Chiang et al. 2019). organoids were grown in a two-dimensional (2D) epithelial monolayer system and exposed on their apical side to air, to obtain a self-organizing monolayer that mimics cells in homeostasis. This monolayer could then be re-submerged in medium (to elicit damage response mimicking in vivo epithelial injuries) and re-exposed to air (which induces epithelial regeneration responses) (FIG. 6H). When treated with IFN-λ the epithelial monolayer after re-exposure to air, the proliferative repair response was curbed, as demonstrated by the failure to incorporate EdU (FIG. 6I).

Overall, the data demonstrate that IFN-λ signaling induces a ZBP-1-GSDMC axis that controls epithelial cell survival, and dampens the capacity of ISCs to proliferate and orchestrate tissue restitution.

### DISCUSSION

It was determined that IFN-λ restrains the restitution of the intestinal mucosae secondary to either inflammatory damage or ionizing radiations toxicity. It was also determined that the capacity of IFN-λ to initiate a previously overlooked molecular cascade in intestinal epithelial cells that allows the induction of ZBP1, the activation of caspases, and the induction and cleavage of GSDMCs. Further, similar pathways are transcriptionally upregulated in IBD patients with active disease. Induction of epithelial cell death via the ZBP1-GSDMC axis reduced the number of ISCs, and dampened the proliferation and restitution of epithelial cells, thus affecting the re-epithelization of the injured intestine. Finally, the results reveal that IFN-λ, but not type I IFNs, are the major drivers of the delayed restitution in vivo in mouse models of gut damage.

The immune system is endowed with the capacity to not only protect against pathogen invasion, but also to maintain tissue homeostasis. Fundamental to exert these activities, is the fine balance between anti-microbial functions of the immune system that can drive tissue damage, and the regenerative capacity of organs and tissues. Many cellular and molecular mediators of the immune system are involved in exerting anti-microbial and potentially damaging functions, but several can also modulate mucosal repair. In this instance, the focus was placed on a group of IFNs, known as type III IFNs or IFN-λ. IFN-λ activities at mucosal surfaces are essential to limit pathogen spread while reducing inflammation and immune cell infiltration (Broggi, Granucci et al. 2020). IFN-λ and type I IFNs regulated very similar transcriptional programs, but the limited expression of the IFNLR restricted the activity of IFN-λ to epithelial cells, hepatocytes, neutrophils and few other cell types and, thus, reduced the extent of the inflammation (Broggi, Granucci et al. 2020). The limited number of cells that responded to IFN-λ signaling, and the reduced capacity of IFN-λ, compared to type I IFNs, to activate IRF1 (Forero, Ozarkar et al. 2019), allow for the preservation of the functionality of mucosal tissues during an immune response. Although the protective functions of IFN-λ in the gut, and in general at mucosal surfaces, are well known (Broggi, Granucci et al. 2020), much less is known about the functions of this group of IFNs during the healing phase that follows intestinal tissue damage. The data reveal the unique capacity of IFN-λ, compared to type I IFNs, to negatively affect tissue restitution in the intestine, possibly opening new ways of therapeutic intervention for individuals that encounter tissue damage such as IBD patients or subjects exposed to radiation therapies.

A common feature revealed by analyses is that IFN-λ affects the survival of the cells, decreases the number of ISCs, and dampens the proliferation of the cells in the crypts, both in vivo in mice and in vitro in both human and mouse organoids. Tissue restitution in the gut is regulated by a complex crosstalk between epithelial cells, immune cells, microbial stimuli, and mesenchymal cells and culminates in the proliferation of ISCs. Treatment with ionizing radiation induces widespread epithelial damage and targets in particular proliferating ISCs in the intestinal crypt, making it an ideal model to understand the dynamics of ISCs proliferation and intestinal healing. Lrg5⁺ ISCs support normal cell turnover as well as injury-induced restitution (Metcalfe, Kljavin et al. 2014). When ISC are depleted by radiation, or by immune-mediated tissue-damaging events, TA cells retro-differentiate and acquire new stem-like properties in the small, as well as in the large, intestine (Wang, Chiang et al. 2019, Ohara, Colonna et al. 2022). These cells then proliferate to allow the re-epithelization of the damaged tissue. The capacity of IFN-λ to dampen lung epithelial cell proliferation (Broggi, Ghosh et al. 2020, Major, Crotta et al. 2020) and to instruct anti-proliferative transcriptional programs in the lung of patients infected with SARS-CoV-2 (Sposito, Broggi et al. 2021) has been explored. Nevertheless, new findings in the gut suggest that decreased proliferation assessed at transcriptional and cellular levels is due to augmented cell death, possibly occurring in newly generated ISCs or in TA cells. If similar processes also take place in the lung, it remains an open question that will require further investigation.

Another interesting observation is that exogenous administration of IFN-λ did not induce caspase or GSDMC activation in vivo in the absence of tissue damage, although it induced ZBP1 upregulation at the transcriptional and protein level. ZBP1 is a Z-DNA binding protein, and is part of the PANoptosome, a multiprotein complex that governs the cell fate (Place, Lee et al. 2021). PANoptosis is a form of cell death that encompasses pyroptosis, apoptosis, and necroptosis. ZBP1 can interact directly or indirectly with proteins that regulate cell death and drive the activation of apoptotic caspases 8, 3 and 7, the necroptosis effector MLKL, or pyroptosis effectors Casp-1, 11 and GSDMD. So far, GSDMC has not been associated with ZBP1 and, or PANoptosis. The data highlight the existence of a ZBP1-GSDMC axis that appeared to be the preferential pathway of cell death that is active during cycles of intestinal epithelial damage and restitution. Upregulation of ZBP1 alone is not sufficient to trigger the full activation of the PANoptosome, which is consistent with the inability to detect toxic effects of IFN-λ in the absence of inflammation or tissue damage. Conversely, ZBP1 can be activated both by binding microbial-derived nucleic acids (Kuriakose, Zheng et al. 2018, Muendlein, Connolly et al. 2021) or by binding host-derived Z-DNA following oxidative damage of the mitochondria (Szczesny, Marcatti et al. 2018). It is, thus, possible that in vivo, under tissue-damaging conditions, either microbiota- or host-derived DNA becomes available to induce the assembly and activation of the PANoptosome downstream of ZBP1. In contrast to the in vivo data, administration of IFN-λ to murine or human intestinal organoids induces the ZBP1-GSDMC axis and drives cell death. It is possible that under the aforementioned in vitro experimental conditions, a “tissue damage” signal, e.g. Z-DNA from dying cells that are differentiating in vitro, is available, thus additional signals may be unnecessary.

Linked to the above-mentioned observations, it was also determined that the ISC compartment was not altered under homeostatic conditions in Vil^{CRE}Ifnlr1^{fl/fl} mice compared to WT mice, suggesting that the basal level of IFNs present during homeostasis (Van Winkle, Peterson et al. 2022) did not affect the normal turnover of gut epithelial cells. Intriguingly, it has been recently shown that IFN-λ-dependent responses at homeostasis are restricted to pockets of mature enterocytes in the small intestine and in the colon (Van Winkle, Peterson et al. 2022). In contrast, when mice are injected with rIFN-λ, or infected with murine rotavirus, a type of virus potently controlled by IFN-λ (Walker, Sridhar et al. 2021), responses to IFN-λ broadly distribute along the epithelial layer. These data, together with new findings, suggest that the compartmentalization of IFN-λ signaling at homeostasis preserves the functions of ISCs and the normal turnover of gut epithelial cells.

The presented models of intestinal damage either of the colon, in the DSS-colitis model, or of the small intestine, in the radiation model, highlight the centrality of IFN-λ and its capacity to delay tissue restitution. Two previous studies suggested that IFN-λ may play an opposite role and favor tissue restitution during colitis (Chiriac, Buchen et al. 2017, McElrath, Espinosa et al. 2021). Nevertheless, both studies were performed by inducing colitis in total Ifnlr1^{-/-} mice and thus they suffer the confounding activity of IFN-λ on neutrophils. The absence of IFN-λ signaling in neutrophils potentiates tissue damage (Broggi, Tan et al. 2017), making it hard to compare the tissue restitution phase with WT mice that start from a different level of damage. Indeed, IFNs were always administered or blocked in the colitis model after the peak of the inflammatory phase. Alternatively, mice deficient for the IFNLR were used in epithelial cells only. Total knock-out mice were solely used in the radiation model in which damage and, or inflammation are not driven by neutrophils but by the ionizing radiations. The compartmentalized activity of IFN-λ in different cell types appears to be, thus, a possible feature of this group of IFNs.

Overall, the data unveiled a new axis between IFN-λ, ZBP1 and GSDMC that governs tissue restitution in the gut and open new perspectives to future therapeutic interventions.

### MATERIALS AND METHODS

Mouse strains. C57BL/6J (Jax 00664) (wild-type), Ifnar1^{-/-} (Jax 028288), Mrp8^{CRE} recombinase (Jax 021614), and Vil^{CRE} recombinase (Jax 004586) mice were purchased from Jackson Labs. C57BL/6 IL-28R^{-/-} (Ifnlr1^{-/-}) mice were provided by Bristol-Myers Squibb. Cells from the intestine of C57BL/6 Zbp1^{-/-} mice were kindly provided by Dr. A Poltorak. Cells from the intestine of B6.129S(Cg)-Stat1tm1D1v (Stat1−/−, JAX 012606) were kindly provide by Dr. S.B. Snapper. The mutant mouse line Ifnlr1^{tm1a(EUCOMM)Wtsi} was provided by the Wellcome Trust Sanger Institute Mouse Genetics Project (Sanger MGP) and its funders (funding and associated primary phenotypic Information, sanger.ac.uk/mouseportal). Mice were housed under specific pathogen-free conditions at Boston Children's Hospital, and all the procedures were approved under the Institutional Animal Care and Use Committee (IACUC) and operated under the supervision of the department of Animal Resources at Children's Hospital (ARCH).

Reagents and antibodies. To treat murine organoids in vitro and for in vivo administration, recombinant mouse IFNλ-2 (rIFN-λ) were used and attached to polyethylene glycol (provided by Bristol-Myers Squibb), mouse recombinant IFN-0 (12401-1; PBL interferonsource), anti-IFN-λ2-3 (MAB17892; R&D systems) and anti-IFNAR1 (BE00241; BioXCell), the pan caspase inhibitor Z-VAD-FMK (HY-16658B; MedChem Express), EdU (NE08701; Carbosynth). To treat human organoids in vitro recombinant human IFNλ-2 (300-02K; Peprotech) was used. The following antibodies were used for immunoblotting: j-Actin (Mouse monoclonal; A5441; AC-15 clone; Lot #127M4866V; Sigma-Aldrich), Rsad2 (Mouse monoclonal custom made; BioLegend) Zbp1 (Mouse monoclonal; AG-20B-0010-c100; Lot #A28231605; AdipoGen), gasderminC-2/-3 (Rabbit monoclonal; 229896; Lot #GR3317481-6; abcam), gasdermin D (Rabbit polyclonal; 20770-1-AP; Proteintech), Caspase 8 (D35G2; 4790; Lot #2; CST), Caspase 3 (9662; Lot #19; CST), cleaved caspase 8 (Rabbit monoclonal; Asp387; D5B2; 8592; Lot #4; CST), cleaved caspase 3 (Rabbit monoclonal; D175; 5A1E; 9664; Lot #22; CST).

DSS-colitis induction. To induce colitis, mice were given 2.5% (w/v) dextran sulfate sodium (DSS, Affymetrix) in drinking water for 7 days and were then administered water for 7 days. Where indicated in the figure legends, mice were received daily intraperitoneal injections of 50 mg kg⁻¹ day⁻¹ rIFN-λ or rIFN-β, and, to deplete endogenous IFN-λ or to block type I IFN signaling, mice received daily intraperitoneal injections of 12.5 mg kg⁻¹ day⁻¹ of anti-IFN-λ2-3 or anti-IFNAR1 antibody respectively. Body weight, stool consistency and the presence of blood in the stool were monitored daily. Weight change was calculated as percentage of initial weight.

Partial body irradiation. Mice were sedated with a mix of ketamine (100 mg/ml) and xylazine (20 mg/ml) intraperitoneally. Mice then received gamma irradiation in a Best Theratronics Gammacell 40 Cesium 137-based irradiator with lead shielding of the head, thorax, and upper extremities to prevent bone marrow failure. In one sitting mice received either 11 Grey of gamma irradiation to assess tissue restitution in small intestinal crypts or 14 Grey of gamma irradiation for survival experiments. To assess proliferation, mice were intraperitoneally administered a 100 mg/kg dose of EdU in saline, final volume 500 μl.

Histological Analysis and Immunofluorescence. For morphologic analysis of irradiation experiments, fragments of the small intestine were fixed overnight in 4% paraformaldehyde (PFA) and embedded in paraffin for sectioning. Intestinal sections were stained with hematoxylin and eosin. Images were acquired with an EVOS M7000 (Thermo Fisher Scientific). Intact crypts were counted in 3 sections per animal blindly with ImageJ software. Proliferation was assessed by measuring the number of EdU⁺ cells per intestinal crypt. A minimum of 10 crypts per section and 3 sections per mouse were evaluated. For Immunofluorescence analysis, paraffin sections were deparaffinized with sequential washes in xylene and ethanol. Deparaffinized sections were then stained for EdU incorporation. All quantifications were executed in a blinded fashion.

For histology of DSS-colitis experiments, colons were flushed with PBS, flattened and rolled into a ‘Swiss roll’. Colon rolls were fixed in 10% formalin (Fisher Scientific), dehydrated in 70% Ethanol and embedded in paraffin. Paraffin sections were stained with hematoxylin and eosin and histological features were evaluated. Histological scoring was performed in a blinded fashion by assignment of a score of 1-5 to segments of the colon roll (1, presence of leukocyte infiltrate, loss of goblet cells; 2, bottom third of the crypt compromised; 3, two thirds of the crypt compromised; 4, complete crypt architecture loss; 5, complete crypt loss and lesion of the epithelial layer). Each segment was then measured with ImageJ software, and the final histological score of each sample was obtained by ‘weighting’ the score of each segment against the length of the segment and divided by the total length of the colon roll.

EdU incorporation staining. Deparaffinized slides or organoids fixed on transwell were stained for 30 minutes with 2 mM Sulfo-Cyanine5-azide (Lumiprobe) in the presence of 1 mM CuSO4 and 2 mg/ml Sodium ascorbate, in PBS. After EdU staining, slides were stained with DAPI (Sigma) to detect nuclei, and mounted with ProLongGold antifade reagent (Thermo Fisher Scientific).

Crypt extraction. Small intestines were longitudinally cut and rinsed in PBS. Mucus was washed away by incubation with 1 mM DTT at 4 °C for 5 minutes. The tissue was moved to 10 mM EDTA, 1% FBS, 1% sucrose at 37 °C for 5 minutes. Samples were vortexed and small intestine fragments were moved to a new tube with 10 mM EDTA, 1% FBS, 1% sucrose at 37 °C for 10 minutes. Samples were vortexed. The supernatant was filtered through a 70 uM strainer and kept on ice. Small intestine fragments were moved to a new tube with 10 mM EDTA, 1% FBS, 1% sucrose at 37 °C for 10 minutes. Samples were vortexed and the supernatant was combined with the previous fraction. The isolated crypts were resuspended in Trizol for RNA extraction and in RIPA Buffer with protease and phosphatase inhibitors for Western Blot analysis.

Measured cytokine gene expression in the colon, small intestine crypts and organoids. Samples were collected in Trizol (Thermo Scientific) and RNA was isolated using phenol-chloroform extraction. Purified RNA was analyzed for gene expression by qPCR on a CFX384 real-time cycler (Bio-rad) using Power SYBR™ Green RNA-to-C_{T}™ 1-Step Kit (Thermo Scientific, 4389986) and pre-designed KiCqStart SYBR Green Primers (MilliporeSigma) specific for Rsad2 (RM1_Rsad2 and FM1_Rsad2), Lgr5 (RM1_Lgr5 and FM1_Lgr5) and IDT PrimeTime Predesigned qPCR Assays specificfor Gapdh (Mm.PT.39a.1;).

RNA sequencing. For targeted transcriptome sequencing, RNA (15 ng) isolated from small intestinal crypts was retro-transcribed to cDNA using SuperScript VILO cDNA Synthesis Kit (11754-05; Invitrogen). Barcoded libraries were prepared using the Ion AmpliSeq Transcriptome Mouse Gene Expression Panel, Chef-Ready Kit (A36412; Thermo Scientific) as per the manufacturer's protocol. Sequencing, read alignment, de-multiplexing, quality control and normalization was performed using an Ion S5 system (A27212; Ion Torrent). The generated count matrixes were analyzed using custom scripts in R (v 4.1.1). Differential Expression of Gene analysis was performed using the R package DEseq2 (v 1.34) with shrinkage of log 2 fold changes. Volcano plots were created using the R package EnhancedVolcano (v 1.12). The differentially expressed genes with an adjusted p-value lesser that 0.1 and a log 2 fold change greater than 1.5 were selected for downstream analysis. Functional enrichment analysis in Gene Ontology was performed using the R package ClusterProfiler (v 4.2) with the Biological Process terms and Benjamin-Hochberg multi-test correction with 5% of FDR threshold. Geneset Enrichment Analysis (GSEA) of hallmarks was performed using the R package fgsea (v 1.20) using the hallmark genesets (v 7.4) from the Broad Institute MSigDB or custom genesets. Leading edges of the different selected genesets were selected to build heatmaps of their expression in the different conditions and samples. The R package ComplexHeatmap (v 2.10) was used to plot the heatmaps. CIBERSORTx was used (Newman, Steen et al. 2019) to estimate the abundances of epithelial cell types using bulk gene expression data as an input and scRNAseq signature matrices from single-cell RNA sequencing data to provide the reference gene expression profiles of pure cell populations. The scRNAseq signature matrix used to deconvolute RNAseq dataset from small intestine crypts was taken from (Haber, Biton et al. 2017).

Western blot. Western blot was performed with standard molecular biology techniques. Blots were probed for: β-Actin, Rsad2, Zbp1, gasdermin C-2/-3, gasdermin D, caspase 8, caspase 3, cleaved caspase 8 and cleaved caspase 3.

RNAseq on IBD patient's biopsies. The IBD biobank was generated starting with biopsy samples collected from patients suffering from CD or UC and diagnosed as clinically quiescent or in an active phase of the disease with various degrees. Controls were taken from non-inflammatory healthy portions of the colon. The investigation was registered under ClinicalTrials.gov Identifier: NCT02304666. A detailed description of the biobank as well as the RNAseq studies are described elsewhere (V. Millet et al., submitted). All raw and processed sequencing data generated in this study are accessible on the NCBI Gene Expression Omnibus (GEO) under the meta-series GSE174159: ncbi.nlm.nih.gov/geo/query/acc.cgi?acc=GSE174166

Samples were divided in three groups according to the disease status of the patient: control, inactive and active. The mean of genes for the tested hallmarks were computed by mean of their expression across the samples of each group. Linear regression analyses were performed using the geom_smooth function of the R package ggplot (v 3.3.5) with the “lm” method. Correlation analysis were performed by a Spearman test using the cor.test function of the R base package stats.

Bulk RNA-seq sequencing data was downloaded from NCBI GEO for the RISK (ncbi.nlm.nih.gov/geo/query/acc.cgi; TPM-normalized counts matrix) and PROTECT(ncbi.nlm.nih.gov/geo/query/acc.cgi?acc=GSE109142; RPKM-normalized count matrix) cohorts. For the RISK data, samples with “undetermined” histopathology data were excluded from the analysis, and IBD samples labeled as macroscopically or microscopically inflammation were categorized as “Active” with the rest as “Inactive”. For the PROTECT cohort, samples lacking histology scores were excluded from the analysis and all other IBD samples were categorized as “Active” if they had a Histology Severity Score (for chronic and active acute neutrophil inflammation)>1 and “Inactive” if they had a Histology Severity Score of 0-1 (Boyle, Collins et al. 2017). Group comparisons between healthy controls, inactive and active IBD were performed using non-parametric t-testing (Wilcoxon test) and p values reported.

Organoid culture and stimulation. Mouse intestinal spheroids were derived and maintained as previously described (Miyoshi and Stappenbeck 2013). Briefly, 1 cm long segments of the small intestine were incubated in 5 ml of 2 mM EDTA for 30 minutes at 4 °C under rotation. Following the incubation period, the tubes were vigorously shaken, and the supernatant was passed through a 70 m strainer to collect small intestinal crypts and exclude villi fragments. The crypt compartment was collected by centrifugation, washed with advanced DMEM/F12 media (Thermo Fisher Scientific), resuspended in cold Matrigel (Corning) and plated in 40 μl domes with 50% L-WRN (Wnt3, R-spondin, Noggin) supplemented medium.

Human organoids derived from healthy patients' duodenal biopsies were provided by Dr. Jay Thiagarajah. Duodenal biopsy samples were obtained from routine diagnostic endoscopy under Boston Children's Hospital IRB protocol P00027983 and cultured with methods modified from (Sato, Stange et al. 2011). Briefly, crypts were dissociated from duodenal biopsy samples obtained from age-matched (<3 years) healthy control individuals. Isolated crypts were suspended in Matrigel and plated in 50 μL domes with 50% L-WRN supplemented media.

For maintenance, organoids were liberated from the extracellular matrix by incubating in cell recovery solution (Corning) at 4 °C for 30 minutes, then a single cell preparation was obtained by incubating in TrypLE express (Thermo Fisher Scientific) at 37 °C for 5 minutes. Single cells were then re-plated in Matrigel with 50% L-WRN supplemented media and 10 mM Rock inhibitor Y-27632. For western blot experiments, organoids were plated for 6 days. Organoids were then treated as indicated in the figure legends by adding cytokines and reagents to the medium overlaying the organoids. At the indicated timepoints, organoids were liberated from the extracellular matrix and lysed in RIPA buffer (Sigma). For microscopy of 3D organoids, freshly isolated crypts were seeded in 10 μL of extracellular matrix in μ-Slide Angiogenesis (Ibidi) and overlayed with 45 μl 50% L-WRN conditioned medium supplemented with 10 mM Rock inhibitor Y-27632 for 24 hours. After 24 hours organoids were treated according to the figure legend in the presence of 1 ug/ml Propidium Iodide (PI) (Sigma), and incubated in the video microscope “Observer Z.1 Zeiss with Hamamatsu ORCA Flash 4.0 LT”, equipped with a temperature-controlled and CO₂ chamber. Wells were scanned every 12 hours and mosaic brightfield and fluorescence images were taken. Organoids were identified by ImageJ and were followed over time for PI incorporation as hallmark of cell death. Percent of live organoids was expressed as Percent of organoids that never incorporated PI. Where indicated in the figure legends, organoids viability was measured with CellTiter-Blue (Promega) according to the manufacturer's instruction. Percentage of live organoids is expressed based on relative CellTiter-Blue signal compared to untreated organoids.

For experiments with 2D organoids in Air-Liquid Interface (ALI), a previously described protocol was followed (Wang, Chiang et al. 2019). Briefly, cultured mouse small intestinal organoids were dissociated in single cells and seeded on polycarbonate transwells, with 0.4 M pores (CORNING). Initially, cells were seeded in the presence of 50% L-WRN media with 10 M Rock inhibitor Y-27632 in both the lower and the upper chamber. After 7 days, the media was removed from the upper chamber to create an ALI. Cells were maintained in these conditions for 14 days to establish a homeostatic monolayer. The ALI culture was then resubmerged with 200 L 50% L-WRN medium, for 7 days and re-exposed to air for 3 days in the presence or absence of rIFN-λ, as indicated in the figure legends. After 3 days from re-exposure to air, cells were pulsed with 10 M EdU for 2 hours, fixed in 10% formalin and stained for EdU incorporation. Samples were examined using a Zeiss LSM 880 confocal microscope (Carl Zeiss) and data were collected with fourfold averaging at a resolution of 2100×2100 pixels. The percentage of EdU-positive-cells was calculated as the ratio of the number EdU-positive foci and DAPI-positive foci.

Gsdmc-2 and Gsdmc-3 knocked down in small intestine organoids and analyzed by cytofluorimetry. GSDMC knockdown (Gsdmc2, 3^{KD}) stable cell lines were produced using commercially designed lentivirus particles targeting mouse Gsdmc2 (NM_001168274.1) and Gsdmc3 (NM_183194.3) (Origene #HC108542): shRNA HC1008542A-AGTATTCAATACCTATCCCAAAGGGTTCG (SEQ ID NO: 1), HC108542B-AGTTGTGTTGTCCAGTTTCCTGTCCATGC (SEQ ID NO: 2) and scrambled negative control non-effective shRNA (Origene Item no: TR30023). Lentivirus was packaged by co-transfecting shRNA and psPAX2 and pVSVG packaging plasmids into HEK293T cells. Transfection efficiency was monitored by GFP fluorescence, media was changed 24 hours post transfection and lentivirus particles were harvested in cell culture media 72 hours after transfection. Lentivirus particles were concentrated with Lenti-X™ Concentrator (Takara 631232) with the manufacturers protocol. Concentrated particles were resuspended in 100% WRN conditioned media and titers were checked using Lenti-X™ GoStix™ Plus (Takara 631280). Only high titer lenti-particles were used to transduce duodenoids. For transduction, duodenoids were removed from Matrigel with Cell Recovery Solution and dissociated into single cells in Trypsin-EDTA for 10 minutes. Debris was removed by filtering over a 70 uM Cell strainer (Stem Cell Technologies #27260) and single cells isolated at 300RCF for 10 minutes. Single cells were resuspended in 15 ml of Organoid Growth Media+500 ul of concentrated lentivirus particles in a 15 ml conical tube supplemented with 4 ug/mL polybrene. Cells were spin-transduced in a pre-warmed 32C centrifuge in a swinging bucket rotor at 500×g for 1 hour. Organoid pellet was resuspended in matrigel, plated onto a Corning 24 Well plate, and incubated in a 37C+5% CO2 incubator for 2 hours. After two hours 500 ul of organoid growth media was added. Media was changed every other day. Transduction efficiency was assessed by GFP fluorescence, and positively transduced wells were expanded. Vehicle (Veh) treated, scramble shRNA, and Gsdmc2, 3^{KD} duodenoids were treated with 200 ng/ml of rIFN-λ in Organoid Growth Media. After 48 hours duodenoids were removed from Matrigel in Cell Recovery Solution for 1 hour at 4 °C, washed with PBS and resuspended in Organoid FACS Buffer (1× PBS+1% BSA+2 mM EDTA+10 uM Y27632). Duodenoids were stained with Zombie Aqua™ (Biolegend) and Calcein Red™ AM (Thermofisher) in Organoid FACS buffer, diluted according to manufacturer's protocol. Cells were washed twice with PBS and mechanically disrupted by pipetting with a P200 pipet tip, then incubated with prewarmed Trypsin-EDTA for 10 minutes at 37C. After 10 minutes trypsin was quenched with Organoid FACS buffer, cells were spun at 300×g for 10 minutes at 4 °C, and resuspended in Organoid FACS Buffer for flow cytometry. Fluorescent positive gates were positioned relative to vehicle-treated and DMSO treated controls.

Quantification and Statistical Analysis. Statistical significance was assessed by: Unpaired t-test to compare two groups, One-way ANOVA with Dunnett correction for multiple comparisons to compare 3 or more independent groups, Two-way ANOVA with Turkey or Šidak correction for multiple comparisons to compare two groups with two conditions. Kruskal-Wallis test with Dunn's post hoc test was used to compare 3 or more independent groups when data did not meet the normality assumption. Spearman correlation analysis was used to examine the degree of association between two continuous variables. To establish the appropriate test, normal distribution and variance similarity were assessed with the D'Agostino-Pearson omnibus normality test. Statistical analyses were two-sided and performed using Prism9 (Graphpad) software or using custom scripts in R (v 4.1.1) and details are indicated in figure legends. Throughout the paper statistical significancy is defined as follows: ns, not significant (p>0.05); *p<0.05, **p<0.01, ***p<0.001, and ****p<0.0001.

### REFERENCES

1. Blanpain, C. and E. Fuchs (2014). “Stem cell plasticity. Plasticity of epithelial stem cells in tissue regeneration.” Science 344(6189): 1242281.
2. Boyle, B., M. H. Collins, Z. Wang, D. Mack, A. Griffiths, C. Sauer, J. Markowitz, N. LeLeiko, D. Keljo, J. Rosh, S. S. Baker, M. Pfefferkorn, M. Heyman, A. Patel, R. Baldassano, J. Noe, P. Rufo, S. Kugathasan, T. Walters, L. Denson, J. Hyams and P. S. Group (2017). “Histologic Correlates of Clinical and Endoscopic Severity in Children Newly Diagnosed With Ulcerative Colitis.” Am J Surg Pathol 41(11): 1491-1498.
3. Broggi, A., S. Ghosh, B. Sposito, R. Spreafico, F. Balzarini, A. Lo Cascio, N. Clementi, M. De Santis, N. Mancini, F. Granucci and I. Zanoni (2020). “Type III interferons disrupt the lung epithelial barrier upon viral recognition.” Science.
4. Broggi, A., F. Granucci and I. Zanoni (2020). “Type III interferons: Balancing tissue tolerance and resistance to pathogen invasion.” J Exp Med 217(1).
5. Broggi, A., Y. Tan, F. Granucci and I. Zanoni (2017). “IFN-lambda suppresses intestinal inflammation by non-translational regulation of neutrophil function.” Nat Immunol 18(10): 1084-1093.
6. Broz, P., P. Pelegrin and F. Shao (2020). “The gasdermins, a protein family executing cell death and inflammation.” Nat. Rev. Immunol. 20(3): 143-157.
7. Chiriac, M. T., B. Buchen, A. Wandersee, G. Hundorfean, C. Gunther, Y. Bourjau, S. E. Doyle, B. Frey, A. B. Ekici, C. Buttner, B. Weigmann, R. Atreya, S. Wirtz, C. Becker, J. Siebler and M. F. Neurath (2017). “Activation of Epithelial Signal Transducer and Activator of Transcription 1 by Interleukin 28 Controls Mucosal Healing in Mice With Colitis and Is Increased in Mucosa of Patients With Inflammatory Bowel Disease.” Gastroenterology 153(1): 123-138 e128.
8. Danese, S. and C. Fiocchi (2011). “Ulcerative colitis.” N. Engl. J. Med. 365(18): 1713-1725.
9. Forero, A., S. Ozarkar, H. Li, C. H. Lee, E. A. Hemann, M. S. Nadjsombati, M. R. Hendricks, L. So, R. Green, C. N. Roy, S. N. Sarkar, J. von Moltke, S. K. Anderson, M. Gale, Jr. and R. Savan (2019). “Differential Activation of the Transcription Factor IRF1 Underlies the Distinct Immune Responses Elicited by Type I and Type III Interferons.” Immunity 51(3): 451-464 e456.
10. Gunther, C, B. Ruder, I. Stolzer, H. Dorner, G. W. He, M. T. Chiriac, K. Aden, A. Strigli, M. Bittel, S. Zeissig, P. Rosenstiel, R. Atreya, M. F. Neurath, S. Wirtz and C. Becker (2019). “Interferon Lambda Promotes Paneth Cell Death Via STAT1 Signaling in Mice and Is Increased in Inflamed Ileal Tissues of Patients With Crohn's Disease.” Gastroenterology 157(5): 1310-1322 e1313.
11. Haber, A. L., M. Biton, N. Rogel, R. H. Herbst, K. Shekhar, C. Smillie, G. Burgin, T. M. Delorey, M. R. Howitt, Y. Katz, I. Tirosh, S. Beyaz, D. Dionne, M. Zhang, R. Raychowdhury, W. S. Garrett, O. Rozenblatt-Rosen, H. N. Shi, O. Yilmaz, R. J. Xavier and A. Regev (2017). “A single-cell survey of the small intestinal epithelium.” Nature 551(7680): 333-339.
12. Haberman, Y., R. Karns, P. J. Dexheimer, M. Schirmer, J. Somekh, I. Jurickova, T. Braun, E. Novak, L. Bauman, M. H. Collins, A. Mo, M. J. Rosen, E. Bonkowski, N. Gotman, A. Marquis, M. Nistel, P. A. Rufo, S. S. Baker, C. G. Sauer, J. Markowitz, M. D. Pfefferkorn, J. R. Rosh, B. M. Boyle, D. R. Mack, R. N. Baldassano, S. Shah, N. S. Leleiko, M. B. Heyman, A. M. Grifiths, A. S. Patel, J. D. Noe, B. J. Aronow, S. Kugathasan, T. D. Walters, G. Gibson, S. D. Thomas, K. Mollen, S. Shen-Orr, C. Huttenhower, R. J. Xavier, J. S. Hyams and L. A. Denson (2019). “Ulcerative colitis mucosal transcriptomes reveal mitochondriopathy and personalized mechanisms underlying disease severity and treatment response.” Nat Commun 10(1): 38.
13. Haberman, Y., T. L. Tickle, P. J. Dexheimer, M. O. Kim, D. Tang, R. Karns, R. N. Baldassano, J. D. Noe, J. Rosh, J. Markowitz, M. B. Heyman, A. M. Griffiths, W. V. Crandall, D. R. Mack, S. S. Baker, C. Huttenhower, D. J. Keljo, J. S. Hyams, S. Kugathasan, T. D. Walters, B. Aronow, R. J. Xavier, D. Gevers and L. A. Denson (2014). “Pediatric Crohn disease patients exhibit specific ileal transcriptome and microbiome signature.” J Clin Invest 124(8): 3617-3633.
14. Henden, A. S., M. Koyama, R. J. Robb, A. Forero, R. D. Kuns, K. Chang, K. S. Ensbey, A. Varelias, S. H. Kazakoff, N. Waddell, A. D. Clouston, R. Giri, J. Begun, B. R. Blazar, M. A. Degli-Esposti, S. V. Kotenko, S. W. Lane, K. L. Bowerman, R. Savan, P. Hugenholtz, K. H. Gartlan and G. R. Hill (2021). “IFN-lambda therapy prevents severe gastrointestinal graft-versus-host disease.” Blood 138(8): 722-737.
15. Hou, J., R. Zhao, W. Xia, C. W. Chang, Y. You, J. M. Hsu, L. Nie, Y. Chen, Y. C. Wang, C. Liu,W. J. Wang, Y. Wu, B. Ke, J. L. Hsu, K. Huang, Z. Ye, Y. Yang, X. Xia, Y. Li, C. W. Li, B. Shao, J. A. Taner and M. C. Hung (2020). “PD-L1-mediated gasdermin C expression switches apoptosis to pyroptosis in cancer cells and facilitates tumour necrosis.” Nat Cell Biol 22(10): 1264-1275.
16. Julien, O. and J. A. Wells (2017). “Caspases and their substrates.” Cell Death Differ 24(8): 1380-1389.
17. Kim, C. K., V. W. Yang and A. B. Bialkowska (2017). “The Role of Intestinal Stem Cells in Epithelial Regeneration Following Radiation-Induced Gut Injury.” Curr Stem Cell Rep 3(4): 320-332.
18. Kotenko, S. V., G. Gallagher, V. V. Baurin, A. Lewis-Antes, M. Shen, N. K. Shah, J. A. Langer, F. Sheikh, H. Dickensheets and R. P. Donnelly (2003). “IFN-lambdas mediate antiviral protection through a distinct class II cytokine receptor complex.” Nat Immunol 4(1): 69-77.
19. Kovacs, S. B. and E. A. Miao (2017). “Gasdermins: Effectors of Pyroptosis.” Trends Cell Biol 27(9): 673-684.
20. Kuriakose, T. and T. D. Kanneganti (2018). “ZBP1: Innate Sensor Regulating Cell Death and Inflammation.” Trends Immunol 39(2): 123-134.
21. Kuriakose, T., M. Zheng, G. Neale and T.-D. Kanneganti (2018). “IRF1 Is a Transcriptional Regulator of ZBP1 Promoting NLRP3 Inflammasome Activation and Cell Death during Influenza Virus Infection.” J. Immunol. 200(4): 1489-1495.
22. Liberzon, A., C. Birger, H. Thorvaldsdottir, M. Ghandi, J. P. Mesirov and P. Tamayo (2015). “The Molecular Signatures Database (MSigDB) hallmark gene set collection.” Cell Syst 1(6): 417-425.
23. Major, J., S. Crotta, M. Llorian, T. M. McCabe, H. H. Gad, R. Hartmann and A. Wack (2020). “Type I and III interferons disrupt lung epithelial repair during recovery from viral infection.” BioRxiv.
24. McElrath, C, V. Espinosa, J. D. Lin, J. Peng, R. Sridhar, O. Dutta, H. C. Tseng, S. V. Smirnov, H. Risman, M. J. Sandoval, V. Davra, Y. J. Chang, B. P. Pollack, R. B. Birge, M. Galan, A. Rivera, J. E. Durbin and S. V. Kotenko (2021). “Critical role of interferons in gastrointestinal injury repair.” Nat Commun 12(1): 2624.
25. Meizlish, M. L., R. A. Franklin, X. Zhou and R. Medzhitov (2021). “Tissue Homeostasis and Inflammation.” Annu Rev Immunol 39: 557-581.
26. Metcalfe, C, N. M. Kljavin, R. Ybarra and F. J. de Sauvage (2014). “Lgr5⁺ stem cells are indispensable for radiation-induced intestinal regeneration.” Cell Stem Cell 14(2): 149-159.
27. Miyoshi, H. and T. S. Stappenbeck (2013). “In vitro expansion and genetic modification of gastrointestinal stem cells in spheroid culture.” Nat Protoc 8(12): 2471-2482.
28. Muendlein, H. I., W. M. Connolly, Z. Magri, I. Smirnova, V. Ilyukha, A. Gautam, A. Degterev and A. Poltorak (2021). “ZBP1 promotes LPS-induced cell death and IL-1beta release via RHIM-mediated interactions with RIPK1.” Nat Commun 12(1): 86.
29. Newman, A. M., C. B. Steen, C. L. Liu, A. J. Gentles, A. A. Chaudhuri, F. Scherer, M. S. Khodadoust, M. S. Esfahani, B. A. Luca, D. Steiner, M. Diehn and A. A. Alizadeh (2019). “Determining cell type abundance and expression from bulk tissues with digital cytometry.” Nat Biotechnol 37(7): 773-782.
30. Ohara, T. E., M. Colonna and T. S. Stappenbeck (2022). “Adaptive differentiation promotes intestinal villus recovery.” Dev Cell 57(2): 166-179 e166.
31. Pineton de Chambrun, G., L. Peyrin-Biroulet, M. L6mann and J.-F. Colombel (2010). “Clinical implications of mucosal healing for the management of IBD.” Nat. Rev. Gastroenterol. Hepatol. 7(1): 15-29.
32. Place, D. E., S. Lee and T. D. Kanneganti (2021). “PANoptosis in microbial infection.” Curr Opin Microbiol 59: 42-49.
33. Prokunina-Olsson, L., B. Muchmore, W. Tang, R. M. Pfeiffer, H. Park, H. Dickensheets, D. Hergott, P. Porter-Gill, A. Mumy, I. Kohaar, S. Chen, N. Brand, M. Tarway, L. Liu, F. Sheikh, J. Astemborski, H. L. Bonkovsky, B. R. Edlin, C. D. Howell, T. R. Morgan, D. L. Thomas, B. Rehermann, R. P. Donnelly and T. R. O'Brien (2013). “A variant upstream of IFNL3 (IL28B) creating a new interferon gene IFNL4 is associated with impaired clearance of hepatitis C virus.” Nat Genet 45(2): 164-171.
34. Quir6s, M. and A. Nusrat (2018). “Contribution of Wound-Associated Cells and Mediators in Orchestrating Gastrointestinal Mucosal Wound Repair.” Annu. Rev. Physiol. 81(1): 1-21.
35. Roda, G., S. Chien Ng, P. G. Kotze, M. Argollo, R. Panaccione, A. Spinelli, A. Kaser, L. Peyrin-Biroulet and S. Danese (2020). “Crohn's disease.” Nat Rev Dis Primers 6(1): 22.
36. Sato, T., D. E. Stange, M. Ferrante, R. G. Vries, J. H. Van Es, S. Van den Brink, W. J. Van Houdt, A. Pronk, J. Van Gorp, P. D. Siersema and H. Clevers (2011). “Long-term expansion of epithelial organoids from human colon, adenoma, adenocarcinoma, and Barrett's epithelium.” Gastroenterology 141(5): 1762-1772.
37. Sato, T., J. H. van Es, H. J. Snippert, D. E. Stange, R. G. Vries, M. van den Born, N. Barker, N. F. Shroyer, M. van de Wetering and H. Clevers (2011). “Paneth cells constitute the niche for Lgr5 stem cells in intestinal crypts.” Nature 469(7330): 415-418.
38. Schmitt, M., M. Schewe, A. Sacchetti, D. Feijtel, W. S. van de Geer, M. Teeuwssen, H. F. Sleddens, R. Joosten, M. E. van Royen, H. J. G. van de Werken, J. van Es, H. Clevers and R. Fodde (2018). “Paneth Cells Respond to Inflammation and Contribute to Tissue Regeneration by Acquiring Stem-like Features through SCF/c-Kit Signaling.” Cell Rep 24(9): 2312-2328 e2317.
39. Sheppard, P., W. Kindsvogel, W. Xu, K. Henderson, S. Schlutsmeyer, T. E. Whitmore, R. Kuestner, U. Garrigues, C. Birks, J. Roraback, C. Ostrander, D. Dong, J. Shin, S. Presnell, B. Fox, B. Haldeman, E. Cooper, D. Taft, T. Gilbert, F. J. Grant, M. Tackett, W. Krivan, G. McKnight, C. Clegg, D. Foster and K. M. Klucher (2003). “IL-28, IL-29 and their class II cytokine receptor IL-28R.” Nat Immunol 4(1): 63-68.
40. Sposito, B., A. Broggi, L. Pandolfi, S. Crotta, N. Clementi, R. Ferrarese, S. Sisti, E. Criscuolo, R. Spreafico, J. M. Long, A. Ambrosi, E. Liu, V. Frangipane, L. Saracino, S. Bozzini, L. Marongiu, F. A. Facchini, A. Bottazzi, T. Fossali, R. Colombo, M. Clementi, E. Tagliabue, J. Chou, A. E. Pontiroli, F. Meloni, A. Wack, N. Mancini and I. Zanoni (2021). “The interferon landscape along the respiratory tract impacts the severity of COVID-19.” Cell.
41. Sposito, B., A. Broggi, L. Pandolfi, S. Crotta, R. Ferrarese, S. Sisti, N. Clementi, A. Ambrosi, E. Liu, V. Frangipane, L. Saracino, L. Marongiu, F. A. Facchini, A. Bottazzi, T. Fossali, R. Colombo, M. Clementi, E. Tagliabue, A. E. Pontiroli, F. Meloni, A. Wack, N. Mancini and I. Zanoni (2021). “Severity of SARS-CoV-2 infection as a function of the interferon landscape across the respiratory tract of COVID-19 patients.” bioRxiv: 2021.2003.2030.437173.
42. Szczesny, B., M. Marcatti, A. Ahmad, M. Montalbano, A. Brunyinszki, S.-I. Bibli, A. Papapetropoulos and C. Szabo (2018). “Mitochondrial DNA damage and subsequent activation of Z-DNA binding protein 1 links oxidative stress to inflammation in epithelial cells.” Sci. Rep. 8(1):914.
43. Van Winkle, J. A., S. T. Peterson, E. A. Kennedy, M. J. Wheadon, H. Ingle, C. Desai, R. Rodgers, D. A. Constant, A. P. Wright, L. Li, M. N. Artyomov, S. Lee, M. T. Baldridge and T. J. Nice (2022). “A homeostatic interferon-lambda response to bacterial microbiota stimulates preemptive antiviral defense within discrete pockets of intestinal epithelium.” Elife 11.
44. VanDussen, K. L., N. M. Sonnek and T. S. Stappenbeck (2019). “L-WRN conditioned medium for gastrointestinal epithelial stem cell culture shows replicable batch-to-batch activity levels across multiple research teams.” Stem Cell Res 37: 101430.
45. Walker, F. C, P. R. Sridhar and M. T. Baldridge (2021). “Differential roles of interferons in innate responses to mucosal viral infections.” Trends Immunol 42(11): 1009-1023.
46. Wang, Y., I. L. Chiang, T. E. Ohara, S. Fujii, J. Cheng, B. D. Muegge, A. Ver Heul, N. D. Han, Q. Lu, S. Xiong, F. Chen, C. W. Lai, H. Janova, R. Wu, C. E. Whitehurst, K. L. VanDussen, T. C. Liu, J. I. Gordon, L. D. Sibley and T. S. Stappenbeck (2019). “Long-Term Culture Captures Injury-Repair Cycles of Colonic Stem Cells.” Cell 179(5): 1144-1159 ell15.
47. Xi, R., J. Montague, X. Lin, C. Lu, W. Lei, K. Tanaka, Y. V. Zhang, X. Xu, X. Zheng, X. Zhou, J. F. Urban, Jr., K. Iwatsuki, R. F. Margolskee, I. Matsumoto, M. Tizzano, J. Li and P. Jiang (2021). “Up-regulation of gasdermin C in mouse small intestine is associated with lytic cell death in enterocytes in worm-induced type 2 immunity.” Proc Natl Acad Sci USA 118(30).
48. Yui, S., L. Azzolin, M. Maimets, M. T. Pedersen, R. P. Fordham, S. L. Hansen, H. L. Larsen, J. Guiu, M. R. P. Alves, C. F. Rundsten, J. V. Johansen, Y. Li, C. D. Madsen, T. Nakamura, M. Watanabe, O. H. Nielsen, P. J. Schweiger, S. Piccolo and K. B. Jensen (2018). “YAP/TAZ-Dependent Reprogramming of Colonic Epithelium Links ECM Remodeling to Tissue Regeneration.” Cell Stem Cell 22(1): 35-49 e37.
49. Zhang, J. Y., B. Zhou, R. Y. Sun, Y. L. Ai, K. Cheng, F. N. Li, B. R. Wang, F. J. Liu, Z. H. Jiang, W. J. Wang, D. Zhou, H. Z. Chen and Q. Wu (2021). “The metabolite alpha-KG induces GSDMC-dependent pyroptosis through death receptor 6-activated caspase-8.” Cell Res 31(9): 980-997.

### Example 3: Kinase Activity of Ripk1 and Presence of Ripk3 are Fundamental for Activity of Complex Formed Under Damage Conditions

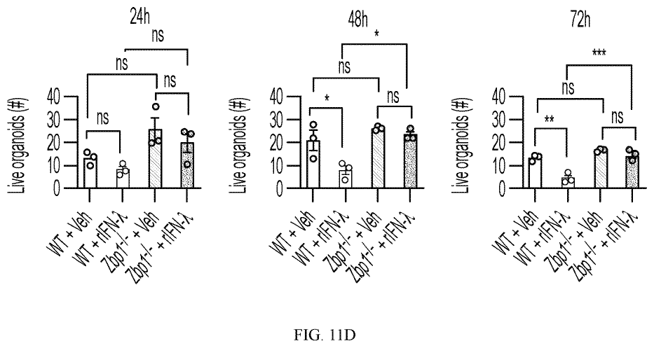

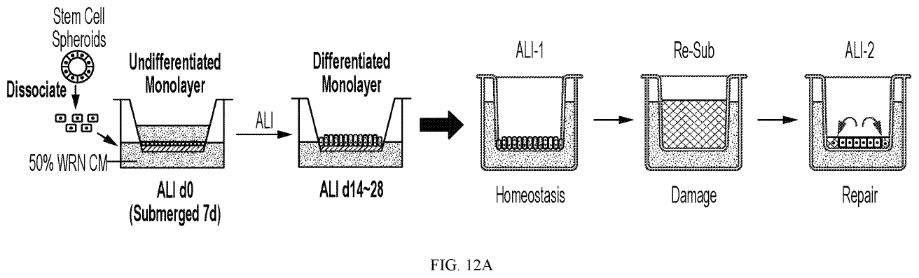

To test whether the kinase activity of Ripk1 and the presence of Ripk3 were needed for the activity of the complex formed upon IFNL encountering damage conditions the following was performed. Small intestine organoids were isolated from wild-type (WT), Ripk1^{D138N} mutant (kinase dead), Ripk3^{-/-} (knockout or “KO”), Ripk3^{-/-}/Caspase 8^{/-} (double knockout or “dKO”). Briefly, the organoids were exposed to 2D Air-Liquid-Interface culture (ALI culture). While in ALI culture, organoids were seeded and submersed for 7 days, followed by 7 days of ALI differentiation. See Wang et al. Cell 2019. To induce hypoxic damage, organoids were re-submerged for 3 days. On day 3 of the re-submersion organoids were treated with IFN-λ (200 ng/ml) for 72 hours total. Controls were not treated with IFN-λ (NT). Organoids were exposed to ALI for 2 days after re-submersion for repair phase in which IFN-λ was still present (FIG. 12A). Immunofluorescent staining and quantification of dead cells (using Zombie Red) and cleaved Caspase 8 (cCasp8) was performed. Under no treatment (NT) conditions, organoids had little to no cells with the presence of cCasp8. When exposed to IFN-λ (200 ng/ml) wild-type (WT) mice exhibited about 30% of cells positive for cCasp8. Relative to organoids derived from WT mice treated with IFN-λ, organoids derived from Ripk1D138^{N} mutant (kinase dead), and Ripk3^{-/-} (knockout or “KO”), and Ripk3^{-/-}/Caspase 8^{/-} (double knockout or “dKO”) mice treated with IFN-λ exhibited a decrease in the percentage of dead cells (FIGS. 12B-12C). Moreover, organoids derived from Ripk1^{D138N} mutant (kinase dead), and Ripk3^{-/-} (knockout or “KO”) mice exhibited a decrease in the percentage of cells positive for cCasp 8.

These data demonstrate that the kinase activity of Ripk1 as well as the presence of Ripk3 are fundamental for the activity of the complex formed upon IFNL encounter under damaging conditions.

### Example 4: Binding Activity of ZBP1 is Fundamental for Activity of Complex Formed Under Damage Conditions

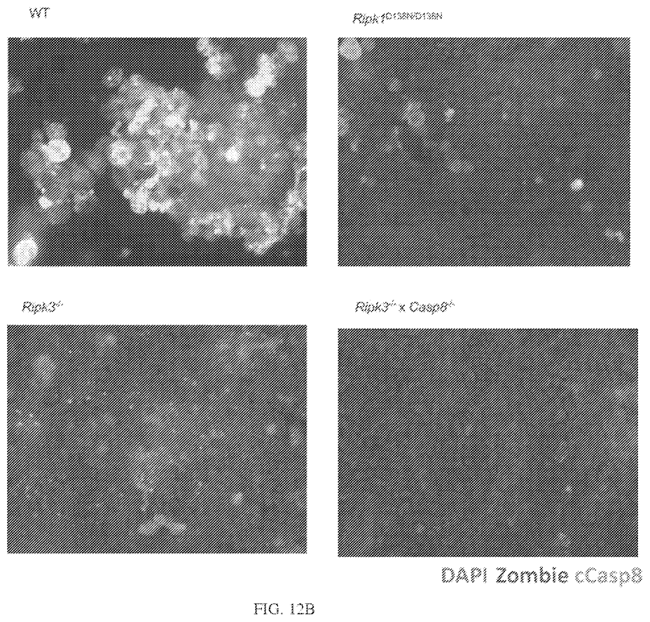

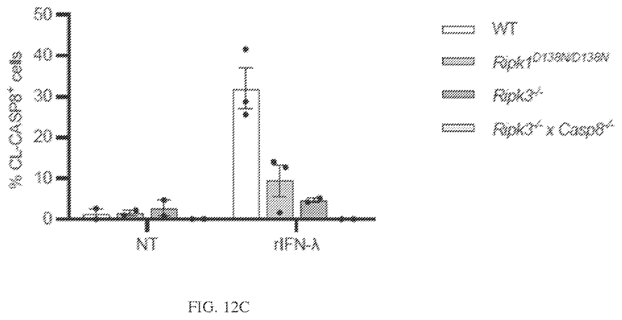

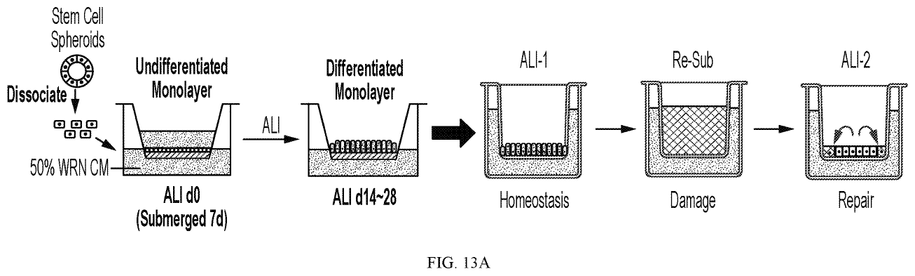

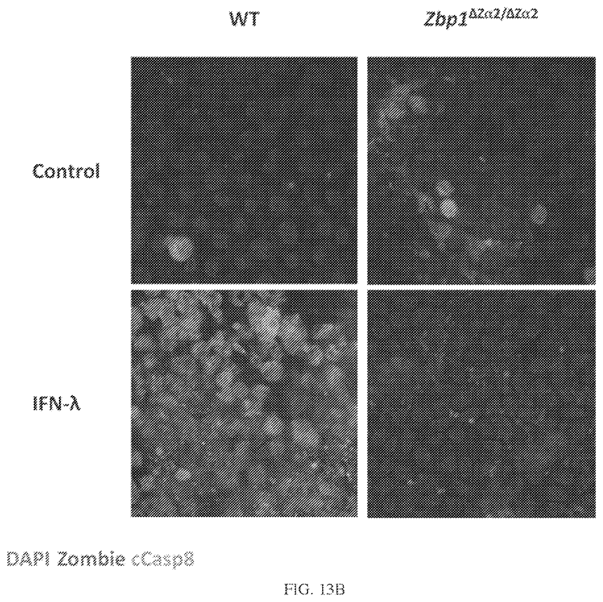

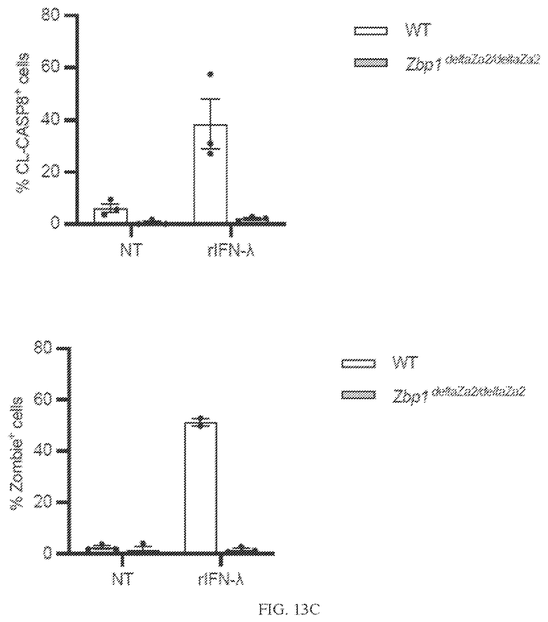

To test whether the binding activity of Zbp1 was needed for the activity of the complex formed upon IFNL encountering damage conditions the following was performed. Colon organoids were isolated from WT, and Zbp1^{ΔZα2/ΔZα2}. Zbp1^{ΔZα2/ΔZα2} mice have a mutation in Zbp1 that prevents Zbp1 from binding to Z-nucleic acids. Briefly, the organoids were exposed to 2D Air-Liquid-Interface culture (ALI culture). While in ALI culture, organoids were seeded and submersed for 7 days, followed by 7 days of ALI differentiation. See Wang et al. Cell 2019. To induce hypoxic damage, organoids were re-submerged for 3 days. On day 3 of the re-submersion organoids were treated with IFN-λ (200 ng/ml) for 72 hours total. Controls were not treated with IFN-λ (NT). Organoids were exposed to ALI for 2 days after re-submersion for repair phase in which IFN-λ was still present (FIG. 13A). Immunofluorescent staining and quantification of dead cells (using Zombie Red) and cleaved Caspase 8 (cCasp8) was performed.

Under no treatment (NT) conditions, organoids had little to no cells with the presence of cCasp8. When exposed to IFN-λ (200 ng/ml) wild-type (WT) mice exhibited about 40% of cells positive for cCasp8. Relative to WT mice treated with IFN-λ, Zbp1^{ΔZα2/ΔZα2} treated with IFN-λ exhibited a decrease in the percentage of cells positive for cCasp8.

Under no treatment (NT) conditions, organoids had little to no cells with the presence of Zombie. When exposed to IFN-λ (200 ng/ml) wild-type (WT) mice exhibited about 50% of cells positive for Zombie. Relative to WT mice treated with IFN-λ, Zbp1^{ΔZα2/ΔZα2} treated with IFN-λ exhibited a decrease in the percentage of cells positive for Zombie (FIGS. 13B-13C).

These data demonstrate that the capacity of ZBP1 to bind Z-nucleic acids (RNA or DNA) formed under damaging conditions is fundamental for the activity of the complex formed upon IFNL encounter.

### Example 5: Type III Interferons Induce Pyroptosis in Gut Epithelial Cells and Impair Tissue Repair Upon Intestinal Injury

### Abstract

Tissue damage and repair are hallmarks of the inflammatory process. Despite a wealth of information on the mechanisms that govern tissue damage, mechanistic insight on how inflammatory mediators affect repair is lacking. Here, it was investigated how interferons influence tissue repair after damage to the intestinal mucosa driven by inflammatory or physical injury. It was determined that type III, but not type I, interferons delay epithelial cell regeneration by inducing the upregulation of ZBP1, Caspase-8 activation, and cleavage of Gasdermin C (GSDMC). Cleaved GSDMC drives epithelial cell death by pyroptosis and delays the re-epithelialization of the large or small intestine after colitis or irradiation, respectively. It was also determined that the ZBP-1/Caspase-8/GSDMC pathway was activated in patients with inflammatory bowel disease. These findings highlight a molecular signaling cascade that affects the outcome of the immune response by delaying tissue repair, which has important implications for human inflammatory disorders.

### INTRODUCTION

The immune system has evolved to protect the host from external or internal threats, as well as to maintain homeostasis of organs and tissues. The skin, lungs, gut, and other mucosae are constantly exposed to microbial and/or physical perturbations and harbor multiple immune and non-immune cells that sense the presence of hostile environmental or endogenous factors and mount a defensive response. The causative agent of this response, the response itself, or both, may lead to tissue damage. Tissue damage sensing by tissue-resident as well as newly recruited cells initiates a complex cascade of cellular and molecular processes to restore tissue functionality and homeostasis or adapt to persistent perturbations (1).

The gastrointestinal tract represents an ideal tissue for exploring the mechanisms underlying the exquisite balance between tissue damage and repair orchestrated by the immune system. In the intestine, immune cells, epithelial cells, and commensal microbes are in a dynamic equilibrium. A monolayer of highly specialized intestinal epithelial cells (IECs) separates the gut lumen from the underlying lamina propria. The interplay between microbiota-derived inflammatory cues and the host cells in the intestine profoundly affects the biology of the gut during homeostasis, inflammation, and damage responses. The delicate equilibrium between the microbiota, the epithelial barrier, and the immune system is lost during the development of inflammatory bowel diseases (IBDs) (2, 3). IBDs are characterized by a heightened inflammatory response, which is accompanied by a breach of the intestinal barrier that compromises mucosal homeostasis. Understanding how immune mediators influence epithelial repair is fundamental for identifying new strategies to promote mucosal healing (4, 5).

It was previously shown that a group of interferons (IFNs), known as type III IFNs or IFN-λ (6-8), limits inflammation in a mouse model of colitis by dampening the tissue-damaging functions of neutrophils (9). IFN-λ, similarly to type I IFNs, plays potent anti-microbial roles (10). However, in contrast to type I IFNs which act systemically because of the broad expression of the type I IFN receptor (IFNAR), the expression of the IFN-λ receptor (IFNLR) is mainly restricted to epithelial cells and neutrophils. The local activity of IFN-λ in mucosal tissues limits the extent of activation of immune cells and is believed to prevent excessive tissue damage, while preserving the anti-microbial functions of IFN-λ (10).

Although it was previously shown that IFN-λ limits intestinal tissue damage (9, 11), the involvement of this group of IFNs during tissue restitution in the gut is controversial. During intestinal inflammation, IFN-λ and type I IFNs have been shown to function in a balanced and compartmentalized manner to favor re-epithelization by inducing the secretion of epithelial growth factors and acting, respectively, on IECs or immune cells resident in the lamina propria (12). IFN-λ has also been proposed to facilitate the proliferation of IECs via Signal Transducer and Activator of Transcription 1 (STAT1) signaling (13) and to partially enhance gut mucosal integrity during graft-versus-host disease (14). In contrast, artificial overexpression of IFN-λ in mice favored the death of Paneth cells (15), a group of cells that can facilitate IEC regeneration (16, 17). In keeping with a possible detrimental role of IFN-λ during an inflammatory response at mucosal surfaces, recently it was demonstrated that IFN-λ delays the proliferation of lung epithelial cells in murine models of persistent viral infections (18, 19), as well as in the lower respiratory tract of COVID-19 patients with severe-to-critical outcomes (20). Notably, IFN-λ and/or the IFNLR were found to be upregulated in IBD patients (13, 15), but it is still debated whether this group of IFNs plays protective or detrimental activities during intestinal inflammation.

Here, the role of IFN-λ during gut repair was dissected and it was determined that IFN-λ initiates a signaling pathway that delays epithelial cell regeneration, secondary to either colitis or radiation damage in mice, and that is also activated in IBD patients.

### RESULTS

### IFN-λ Delays Tissue Repair During Colitis.

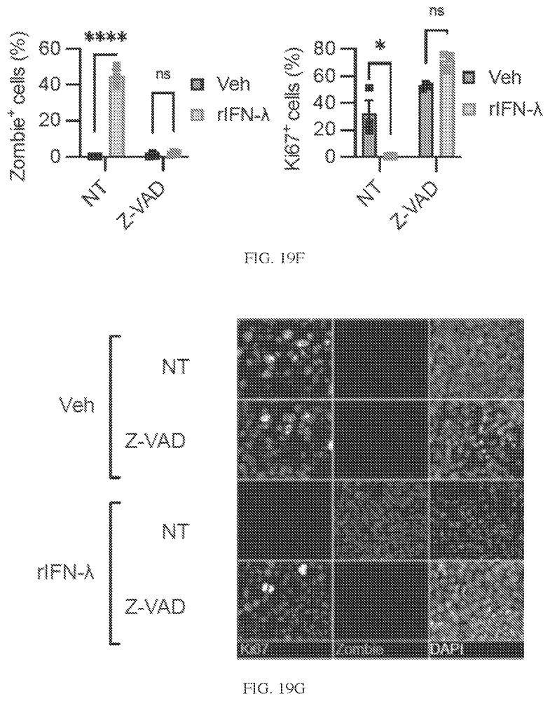

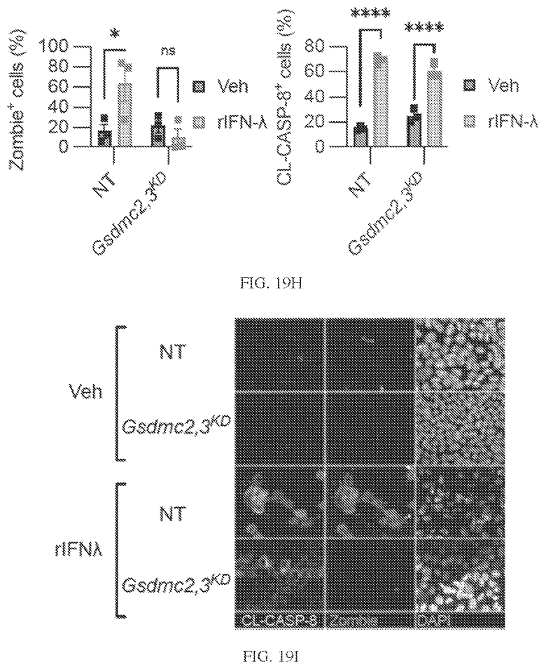

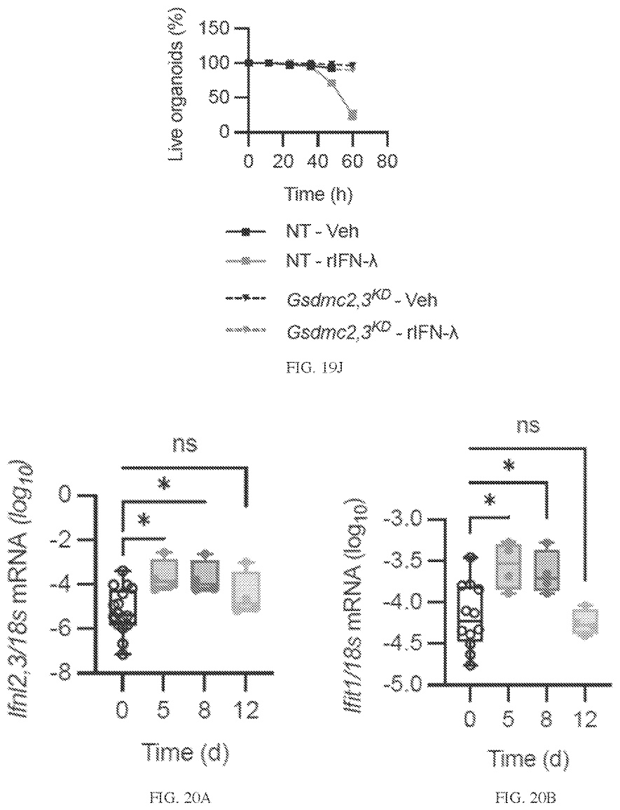

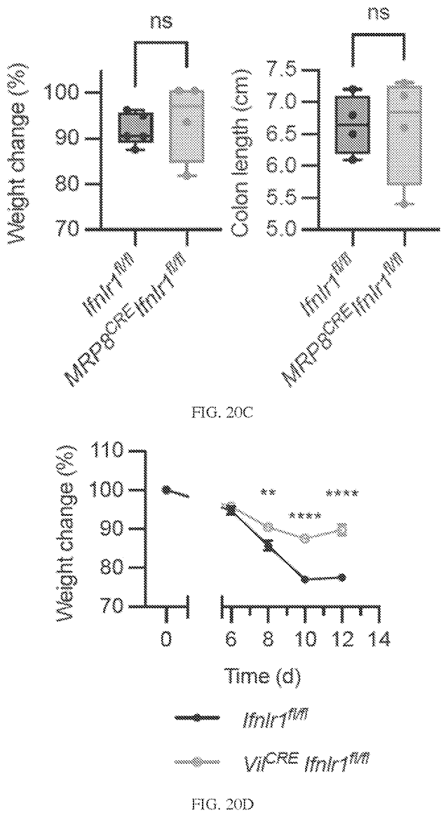

To assess whether IFN-λ can control epithelial regeneration in mice during intestinal inflammation, colitis was induced by the administration of dextran sulfate sodium (DSS). In this murine model of colitis, DSS is administered for 7 days in the drinking water and later removed to allow tissue repair. It was previously showed that, during the first 7 days of acute inflammation upon DSS-administration, IFN-λ signaling in neutrophils, but not in IECs, restrains intestinal damage (9). The measurement of type III IFN gene induction in DSS-treated mice revealed that the levels of IFN-λ, as well as Ifit1, an interferon-stimulated gene (ISG), remain elevated after DSS removal (FIGS. 20A, 20B). These data suggest that IFN-λ can play roles that go beyond the inflammatory phase of DSS-induced colitis.

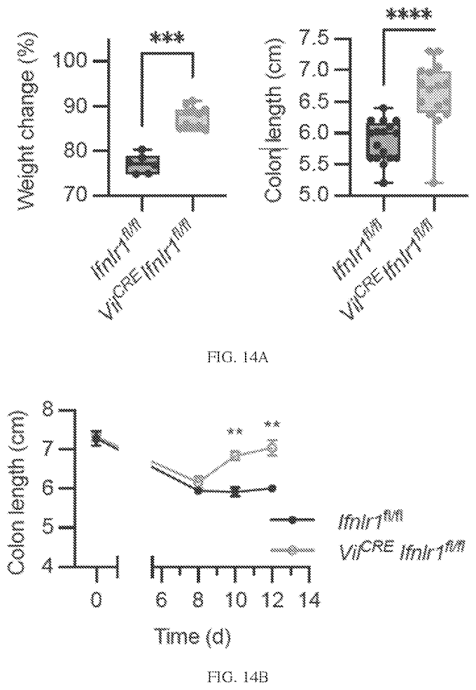

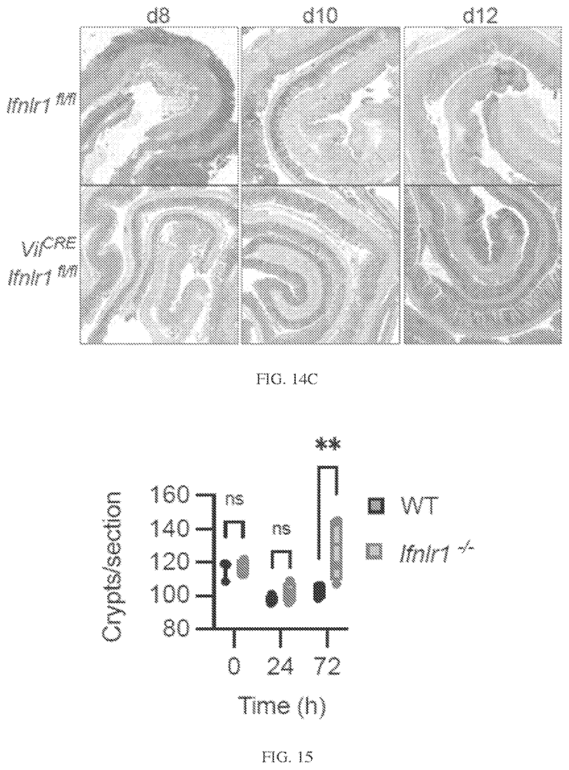

To test the involvement of IFN-λ signaling during the repair phase of colitis, weight loss 10 days after the initial DSS administration was assessed. Mice in which only IECs (Vil^{CRE}Ifnlr1^{fl/fl} mice) or neutrophils (MRP8^{CRE}Ifnlr1^{fl/fl} mice) did not respond to IFN-λ as well as Ifnlr1^{fl/fl} littermate controls were evaluated. It was determined that Ifnlr1^{fl/fl} mice showed a significant decrease in their weight and colon length compared to Vil^{CRE}Ifnlr1^{fl/fl}, but not MRP8^{CRE}Ifnlr1^{fl/fl}, mice (FIGS. 14A, 20C). These data suggested that IFN-λ signaling in IECs delays the repair phase of DSS-induced colitis. Kinetic changes that occur in colon length, as well as tissue damage as measured by histology in Ifnlr1^{fl/fl} littermates and Vil^{CRE}Ifnlr1^{fl/fl} mice was assessed. The data revealed that the colons of mice of both groups were shortened to the same extent at the peak of inflammation (day 8), demonstrating that they were equally affected by DSS-induced acute inflammation (FIG. 14B). Conversely, mice with IECs that respond to IFN-λ showed a significant delay in the recovery of colon length compared to Vil^{CRE}Ifnlr1^{fl/fl} mice (FIG. 14B). The Vil^{CRE}Ifnlr1^{fl/fl} mice returned to a colon length that was similar to the one of untreated animals by day 12 after DSS administration, indicating a complete repair of their intestine (FIG. 14B). In keeping with the capacity of IFN-λ to delay tissue repair, it was determined that similar trends were followed for tissue damage and weight loss (FIGS. 14C, 20D, 20E). Overall, these data demonstrate that endogenous IFN-λ signaling in IECs leads to delayed tissue repair during DSS-induced colitis.

Next, recombinant (r)IFN-λ was injected into mice after DSS-induced inflammation peaked and was compared to a group of mice that received no injection. It was determined that rIFN-λ administration further upregulated ISGs in the colon of DSS-treated mice compared to mice treated with DSS only (FIG. 7A). Mice administered rIFN-λ, but not vehicle controls, showed persistent weight loss, reduced colon length, and more severe tissue damage at late time points as measured by histology (FIGS. 2B, 2C, 2D). These data show that exogenous administration of rIFN-λ further exacerbates the activity of IFN-λ produced by the host during intestinal inflammation.

Next, the relative contribution of type I and type III IFNs to gut repair that follows DSS-induced colitis was tested by either using blocking antibodies directed against IFN-λ or the IFNAR, or by administering rIFN-λ or rIFN-λ after the peak of the inflammatory process induced by DSS administration. In keeping with the data in mice lacking the IFNLR in IECs, the inhibition of endogenous IFN-λ facilitated tissue repair, while addition of exogenous rIFN-λ delayed it, as measured by colon length and weight change (FIGS. 2D, 2E). In contrast to type III IFNs, none of the treatments aimed at targeting type I IFNs affected tissue repair (FIG. 2D). It was also determined that ISG levels in IECs were significantly decreased in mice treated with the anti-IFN-λ antibody (FIG. 7A), further supporting a major role for IFN-λ, rather than type I IFNs, in IECs during tissue repair. Accordingly, ISG levels in colonocytes were not altered in mice that received either the anti-IFNAR antibody or rIFN-λ, compared to control mice (FIG. 7B), confirming previous studies showing that IECs are hyporesponsive to type I IFNs (12, 21). Finally, Vil^{CRE}Ifnlr1^{fl/fl}, but not MRP8^{CRE}Ifnlr1^{fl/fl}, mice were resistant to the administration of exogenous rIFN-λ, confirming the compartmentalized activity of IFN-λ during tissue repair (FIGS. 2H, 2I).

Overall, these data demonstrate that, in contrast to the acute inflammatory phase of colitis, IECs, and not neutrophils, are the major responders to IFN-λ during recovery and that endogenous, as well as exogenous IFN-λ signaling in epithelial cells delays tissue repair.

### IFN-λ Acts on IECs and Delays Tissue Repair Upon Irradiation.

The data in the DSS-induced colitis model demonstrated that IFN-λ acts on IECs to delay repair. To obtain mechanistic insights into the activity of IFN-λ on IECs, a well-characterized model of epithelial damage resulting from exposure to ionizing radiation was employed (22). The repair of the gut epithelial monolayer is a complex process, and the regenerative capacity of intestinal stem cells (ISCs) plays a role (23). After targeted irradiation, ISC death in the small intestine is followed by the repair of the damaged epithelial crypts and the return to homeostasis.

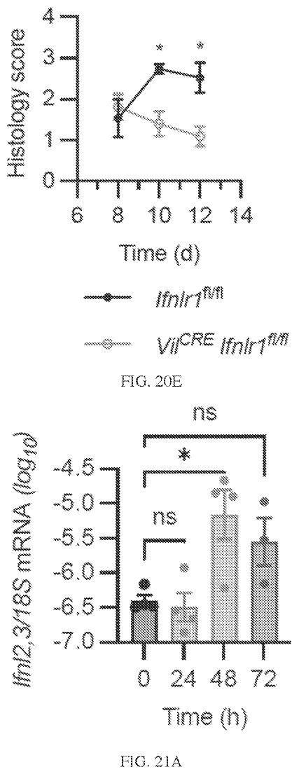

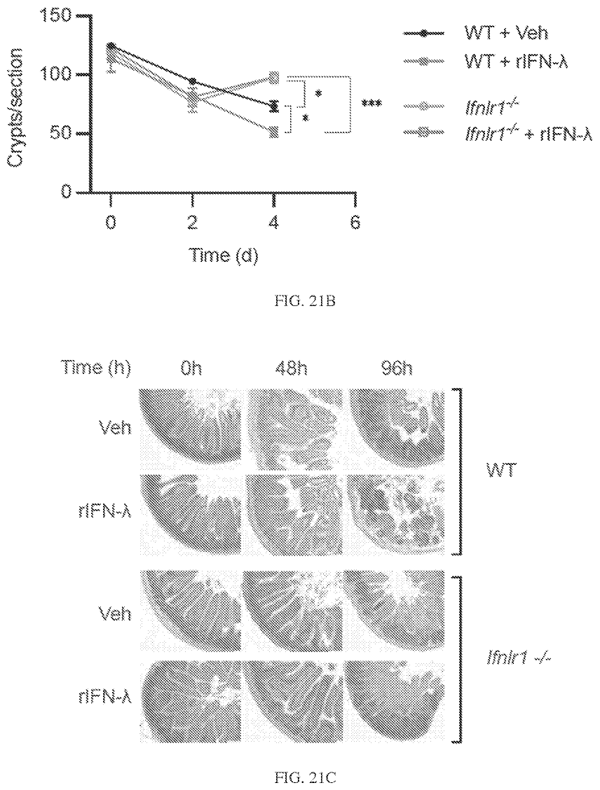

Initially, it was assessed whether IFN-λ was induced upon irradiation and found that the levels of type III IFNs increase over time peaking at 48-72 hours after radiation injury (FIG. 21A). Crypt regeneration was assessed in wild-type (WT) mice, WT mice administered exogenous rIFN-λ, or Ifnlr1^{-/-} mice 96 hours after radiation injury, during the peak of the repair response. It was determined that mice that received rIFN-λ showed reduced regeneration of the crypts, while Ifnlr1^{-/-} mice had an increased number of crypts (FIG. 3A). While the number of crypts 1 day post irradiation was similar in WT and Ifnlr1^{-/-} mice, 3 days after challenge WT mice showed a significant delay in repair compared to Ifnlr1^{-/-} mice (FIG. 3A). Irradiated WT and Ifnlr1^{-/-} mice, administered or not rIFN-λ, revealed that both exogenous and endogenous IFN-λ signaling impairs crypt regeneration (FIGS. 3B, 21B, 21C). Further, endogenous IFN-λ, but not type I IFN, signaling caused the delay in tissue regeneration (FIG. 3B). Similarly, ISG induction in epithelial cells was dependent on IFN-λ, rather than type I IFNs (FIG. 8A).

In keeping with the targeted activity of IFN-λ on IECs, the number of crypts was significantly decreased in WT mice compared to Vil^{CRE}Ifnlr1^{fl/fl} mice, either in the presence or absence of rIFN-λ (FIG. 3C). A role for IFN-λ signaling in neutrophils during the repair process that followed irradiation was excluded using MRP8^{CRE}Ifnlr1^{fl/fl} mice and no significant differences in crypt numbers compared to their WT littermates was observed (FIG. 8C).

Finally, the irradiation was increased to a dose that killed half of the WT mice (LD₅₀) (FIG. 3E). Irradiated Vil^{CRE}Ifnlr1^{fl/fl} mice or Ifnlr1^{fl/fl} littermates, treated or not with rIFN-λ, were followed over time. It was determined that Ifnlr1^{fl/fl} littermates mice irradiated and treated with rIFN-λ lost significantly more weight than irradiated Vil^{CRE}Ifnlr1^{fl/fl} mice, and all mice reached humane endpoints and were sacrificed (FIGS. 3D, 3E). Notably, Ifnlr1^{fl/fl} littermates lost significantly more weight compared to Vil^{CRE}Ifnlr1^{fl/fl} mice, treated or not with rIFN-λ (FIG. 3D). In contrast, in Vil^{CRE}Ifnlr1^{fl/fl} mice, survival rate and weight loss were not significantly affected by treatment with rIFN-λ (FIGS. 3D, 3E).

Overall, these data demonstrate that epithelial cell regeneration and tissue repair in the small intestine of irradiated mice were inhibited in the presence of IFN-λ. In addition, IFN-λ delays repair by acting on IECs.

### IFN-λ Dampens Regenerative and Proliferative Transcriptional Programs in IECs.

To determine the transcriptional programs initiated by IFN-λ to delay tissue repair, intestinal crypts were isolated from the small intestine of Vil^{CRE}Ifnlr1^{fl/fl} mice or WT littermates that have been irradiated and performed targeted RNA sequencing (RNAseq). Gene ontology (GO) enrichment analyses were performed on differentially expressed genes (DEG) overexpressed in WT, compared to Vil^{CRE}Ifnlr1^{fl/fl} epithelial cells (FIG. 4A), or vice versa (FIG. 4A). In keeping with a major role of IFN-λ-dependent responses in the intestine, IFN-signaling related pathways and anti-viral or anti-bacterial pathways were highly enriched in WT epithelial cells, compared to Vil^{CRE}Ifnlr1^{fl/fl} (FIG. 4A). In contrast, GO terms associated with cell migration and extracellular remodeling, linked to higher efficiency in the closure of mucosal wounds (24), were enriched in epithelial cells that do not respond to IFN-λ (FIG. 4A).

A volcano plot analysis of DEG showed enrichment of ISGs in WT, compared to knock-out, cells (FIG. 5A). Gene set enrichment analysis (GSEA) confirmed that the IFNLR controlled the expression of genes associated with IFN responses (FIG. 4D, 9A). These data demonstrated the central role of IFN-λ in governing IFN responses in IECs. Next, the relative enrichment of a previously identified gene-set associated with regenerative epithelium during colitis was assessed (25), as well as a gene-set associated with epithelial cell proliferation. Both gene-sets were significantly enriched when epithelial cells did not respond to IFN-λ (FIGS. 4D, 9B, 9C).

During tissue repair that follows radiation damage (26) or colitis (27), specialized ISCs drive re-epithelialization by massively proliferating. To assess whether endogenous IFN-λ affected the cellular composition of the small intestine in WT or Vil^{CRE}Ifnlr1^{fl/fl} mice that were irradiated, CIBERSORTx (28) was used and the bulk RNAseq data was deconvoluted based on previously published single-cell RNAseq data (29). The deconvolution analysis revealed that, while most epithelial cell types did not present significant differences between irradiated WT and Vil^{CRE} Ifnlr1^{fl/fl}, the ISC compartment was significantly contracted in WT, compared to Vil^{CRE}Ifnlr1^{fl/fl}, mice (FIG. 4F). Decreased cell proliferation upon irradiation was confirmed in vivo in WT, compared to Vil^{CRE}Ifnlr1^{fl/fl}, mice, in the presence or absence rIFN-λ (FIG. 4E).

In keeping with retro-differentiation of somatic cells to replenish the emergency stem cell compartment upon ISC depletion that occurs during colitis (30, 31), after irradiation (32), or in a genetic model (33), it was found that, upon irradiation, transit amplifying (TA) cells were significantly increased in the small intestine of WT, compared to Vil^{CRE}Ifnlr1^{fl/fl} (FIG. 4F). The contraction of the Lrg5⁺ compartment in mice that respond to IFN-λ was confirmed by qPCR (FIG. 9D). It was also determined that the major epithelial cell populations analyzed were not different in homeostasis in Vil^{CRE}Ifnlr1^{fl/fl} mice or WT littermates (FIG. 9E). Overall, these data demonstrate that IFN-λ initiates a transcriptional program that reduces tissue restitution, limits ISCs expansion, and, thus, dampens the overall capacity of epithelial cells to proliferate.

### IFN-λ Controls the Expression of Z-DNA Binding Protein 1 (ZBP1) and the Activation of Gasdermin C (GSDMC) Upon Irradiation Damage or During Colitis.

The reduced expansion of ISCs can be driven by increased cell death of bona fide ISCs and/or retro-differentiating cells that give rise to emergency stem cells. To determine the molecular mechanisms regulated by IFN-λ to dampen tissue restitution, the genes differentially regulated in epithelial cells derived from irradiated WT were identified and compared to Vil^{CRE}Ifnlr1^{fl/fl} mice (FIG. 5A). It was determined that Zbp1, an important regulator of cell fate that has been shown to govern pyroptosis, apoptosis, as well as necroptosis (34), was among the ISGs significantly upregulated by IFN-λ in WT, but not Vil^{CRE}Ifnlr1^{fl/fl}, IECs (FIG. 5A). It was also determined that protein levels of ZBP1, as well as another ISG, RSAD2 (also known as Viperin), were upregulated in epithelial cells of the small intestine upon in vivo administration of rIFN-λ in non-irradiated WT mice (FIG. 10A). Upregulation of these proteins was prevented in epithelial cells derived from Vil^{CRE}Ifnlr1^{fl/fl} mice and was not different in the absence of rIFN-λ in the two backgrounds (FIG. 10A). Among other genes significantly upregulated only in cells that respond to IFN-λ, there were two members of the GSDMC family, Gsdmc2 and Gsdmc3. GSDMs are critical effectors of pyroptosis, a form of inflammatory cell death (35, 36). Some reports have involved GSDMC in the lytic death of tumor cells (37, 38), or of enterocytes during helminth infections (39, 40), but very little is known in the activity of GSDMC during intestinal tissue damage and repair. Resting mice treated with rIFN-λ do not show upregulation of the GSDMC-2/-3 protein (FIG. 10A), demonstrating that additional pathways associated with irradiation and/or tissue damage and repair regulate Gsdmc gene expression and/or protein synthesis.

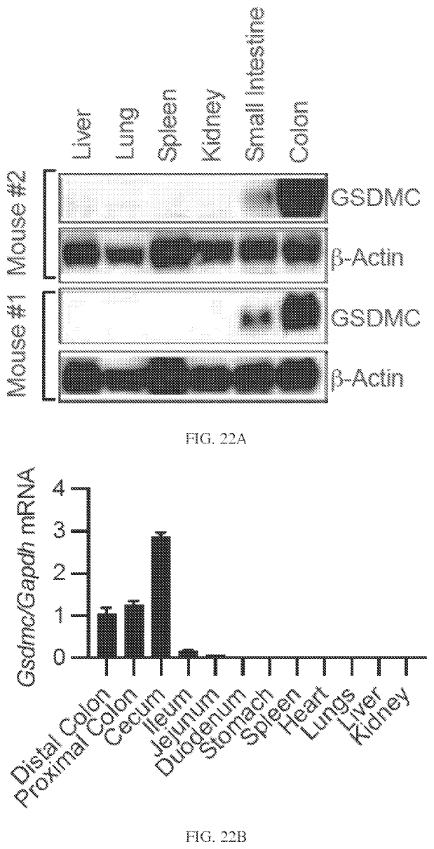

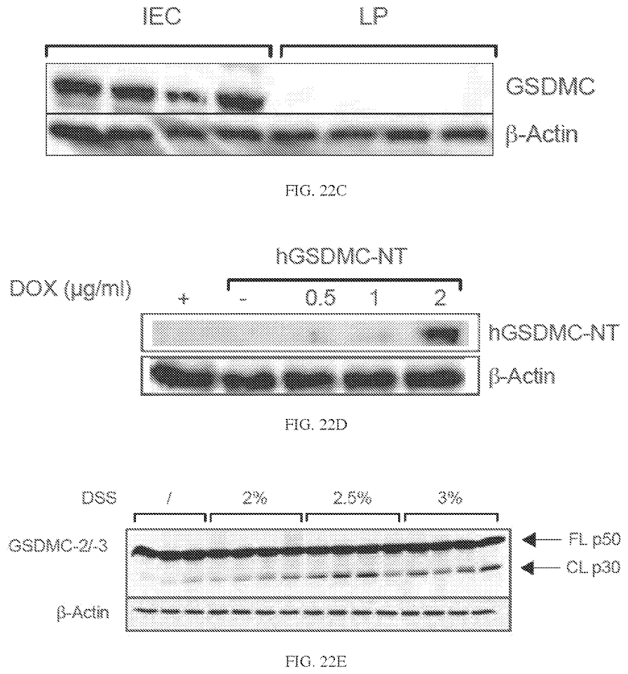

Next the protein expression of GSDMC in different organs under homeostasis was assessed and it was determined that GSDMC was expressed exclusively along the intestinal tract, both in the small and large intestine (FIG. 22A). qPCR analyses of Gsdmc confirmed expression along the gastrointestinal tract, but not in other organs (FIG. 22C). It was also determined that in the colon, GSDMC was present in IECs, but not in the lamina propria (FIG. 22C).

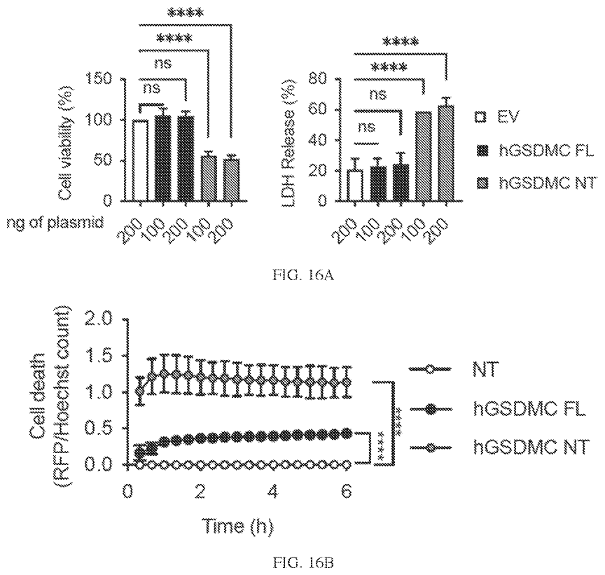

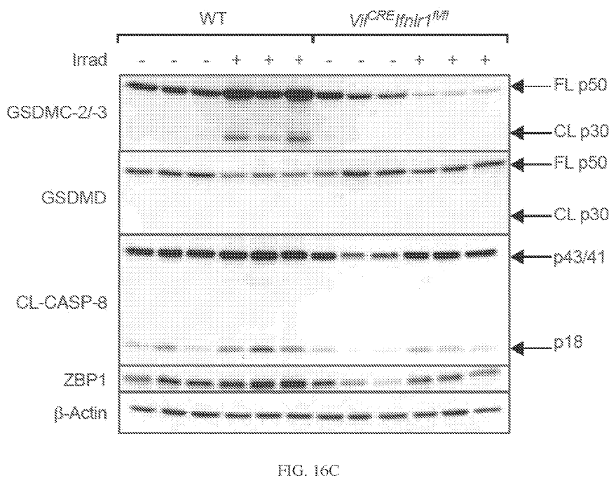

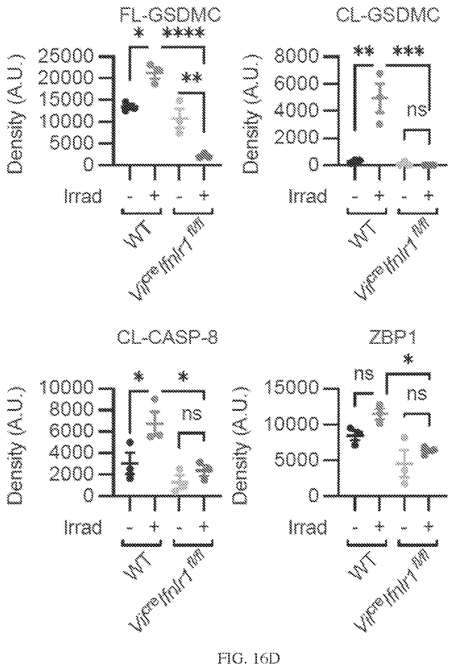

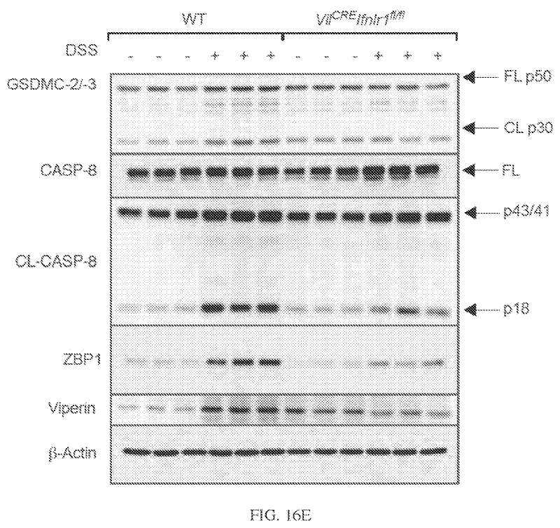

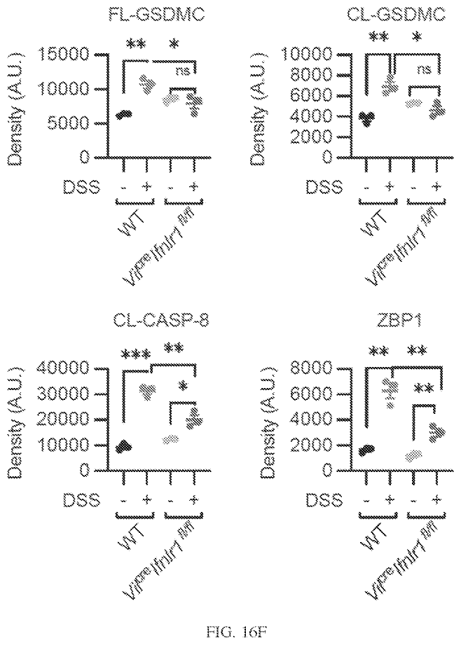

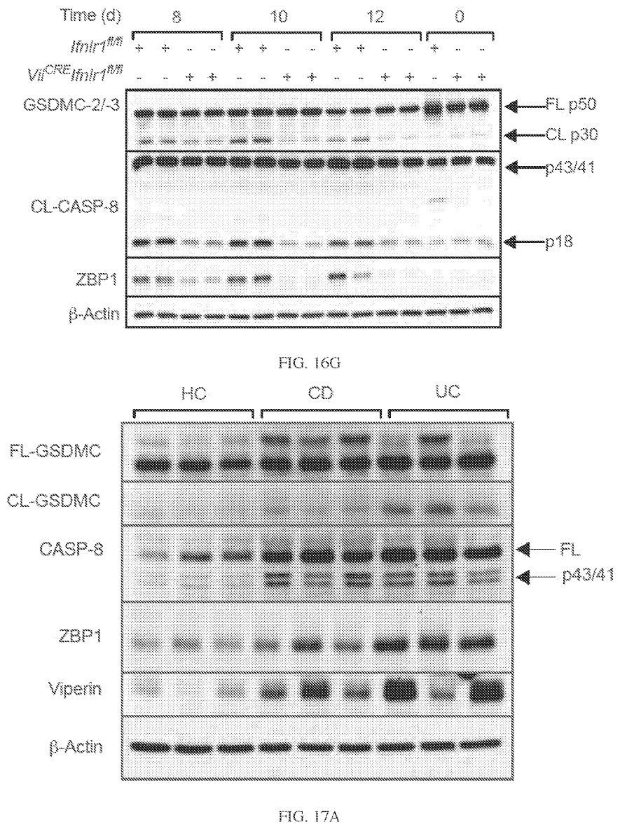

GSDMs exert their pyroptotic function upon cleavage by caspases, when the N-terminal cleavage product oligomerizes to form lytic pores in the cell membrane, leading to the loss of ionic homeostasis and cell death (35, 36). Thus, it was assessed whether the N-terminal domain of GSDMC induces cell death when ectopically expressed. The data demonstrated that the N-terminus of GSDMC was sufficient to induce the lytic cell death of cells when constitutively expressed, and/or induced, in HEK293T cells (FIGS. 16A, 16B, 22D).

Next, it was determined whether GSDMC-2/-3 was cleaved in epithelial cells of the small intestine upon irradiation and confirmed that irradiated, but not non-irradiated, mice not only showed increased levels of ZBP1 and GSDMC-2/-3, but also that GSDMC-2/-3 was efficiently cleaved in WT, but not Vil^{CRE}Ifnlr1^{fl/fl}, mice (FIGS. 16C, 16D). In contrast, another key effector of pyroptosis, GSDMD, was not activated (FOG. 16C).

In keeping with the findings in irradiated mice, it was determined that the ZBP-1/GSDMC-2/-3 axis was also activated in DSS-treated mice. In particular, ZBP-1 and GSMDC-2/-3 were upregulated in WT littermates, but not in Vil^{CRE}Ifnlr1^{fl/fl} mice, and the canonical ISG Viperin followed a similar trend (FIGS. 16E, 16F). Additionally, GSDMC-2/-3 was cleaved in response to IFN-λ signaling (FIGS. 16E, 16F), and GSDMC-2/-3 cleavage was proportional to the extent of the inflammatory response (FIG. 22E). In agreement with the kinetic of IFN-λ induction and response during DSS-driven colitis, it was determined that GSDMC-2/-3 cleavage, and ZBP1 upregulation, peaked between day 8 and 10 after DSS administration (FIG. 16G).

GSDMC family proteins are primarily cleaved by Caspase-8 (Casp-8) (37, 38). Indeed, the pattern of bands of cleaved GSDMC-2/-3 in irradiated mice or mice with colitis is compatible with the activity of Casp-8 (41). Thus, the activation of Casp-8 was investigated, and it was determined that Casp-8 was not activated in WT or Vil^{CRE}Ifnlr1^{fl/fl} mice in the absence of stimulation (FIG. 10A). In contrast, epithelial cells derived from irradiated or DSS-treated WT littermates, but not Vil^{CRE}Ifnlr1^{fl/fl} mice, efficiently activated Casp-8 (FIGS. 16C-16G).

Overall, these data demonstrate that, upon irradiation damage or following colitis induction, IFN-λ initiates a signaling cascade that allows the upregulation of ZBP1, the activation of Casp-8, and the induction and cleavage of GSDMC, an executor of pyroptosis.

### The ZBP-1/Casp-8/GSDMC Pathway is Active in IBD Patients.

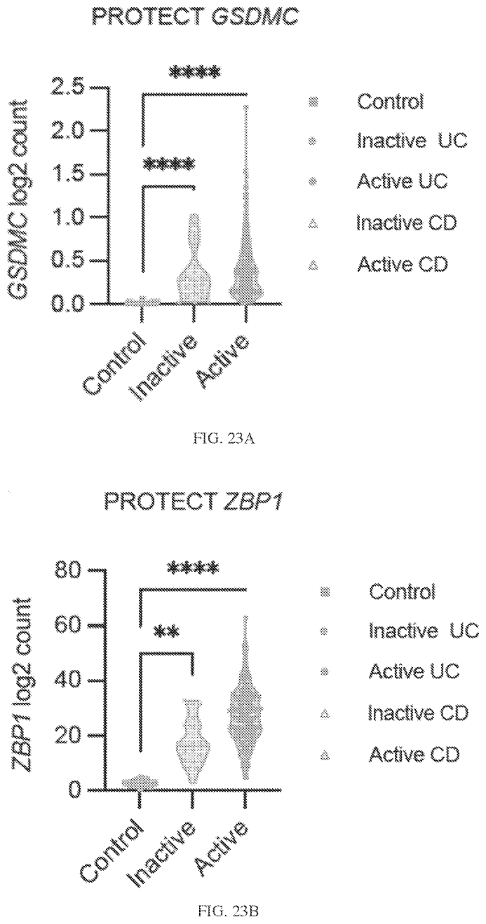

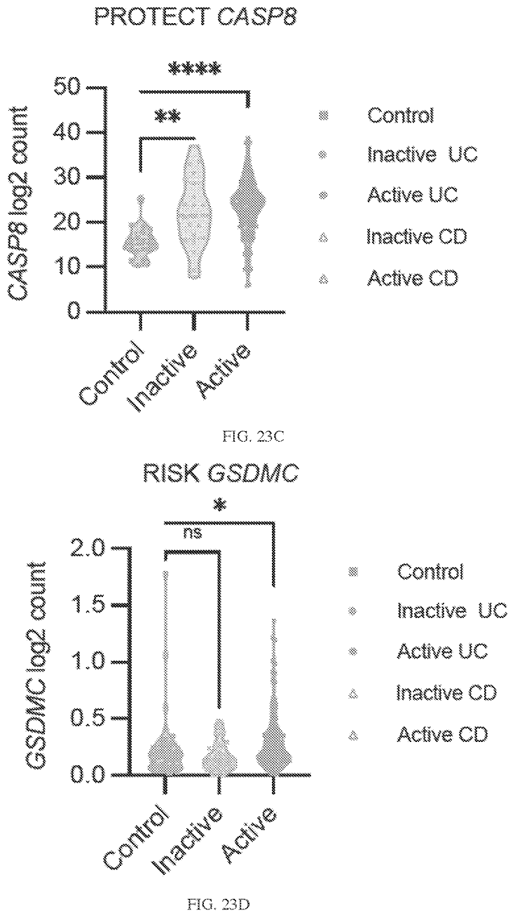

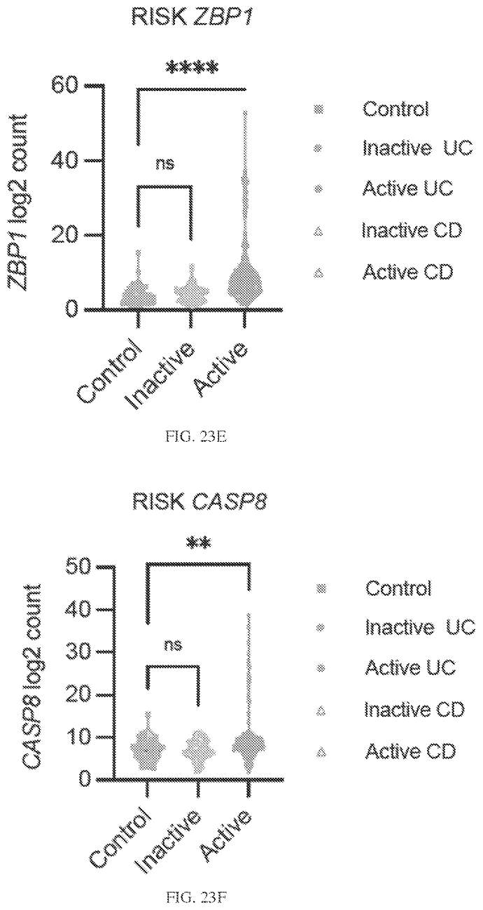

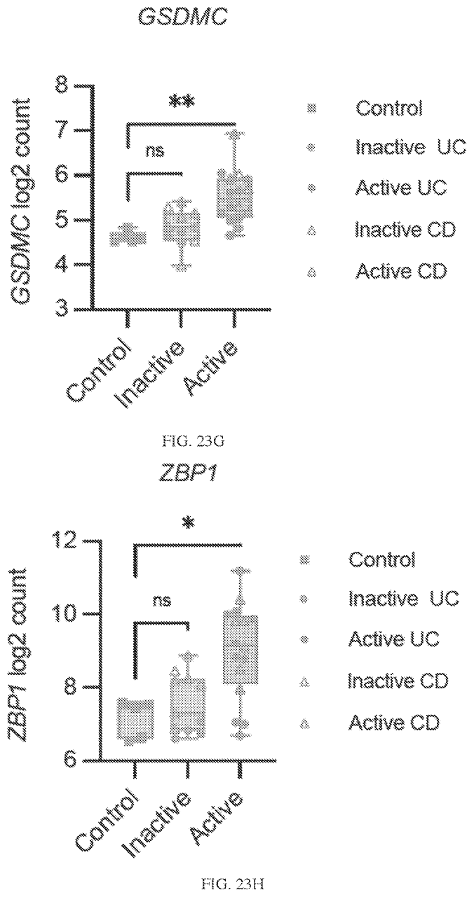

IBD patients present increased levels of IFN-λ and/or IFNLR1 (13, 15). Prompted by THE findings in mice, the expression levels of ZBP1 and GSDMC in RNAseq datasets derived from samples of three different cohorts of patients were assessed, and in particular, from: i) rectal mucosal biopsies from ulcerative colitis (UC) pediatric patients (PROTECT cohort) (42) (FIGS. 23A-C); ii) from ileal biopsies from Crohn's disease (CD) pediatric patients (RISK cohort) (43) (FIG. 23D); and iii) from biopsies derived from a cohort of adult IBD patients with active or inactive disease, or non-IBD controls (44) (FIG. 23G). In all cohorts, a significant increase in the expression of ZBP1 as well as GSDMC in IBD patients was found when, compared to non-IBD controls, with active patients having the strongest and most significant upregulation (FIGS. 23A-23B, FIGS. 23D-23E, FIG. 23G, and FIG. 23H). Further analyses in the adult cohort also revealed an association between GSDMC expression and IFN responses (FIG. 5C). It was determined that the expression of CASP8 was increased in patients with active IBD compared to non-IBD controls (FIG. 23C, FIG. 23F, and FIG. 23I). To further dissect whether the ZBP-1/Casp-8/GSDMC pathway was active in IBD patients also at the protein level, the biopsies from non-IBD individuals, and UC or CD pediatric patients were compared. It was determined that ZBP-1 and GSDMC proteins were significantly upregulated, and GSDMC and Casp-8 were cleaved, in patients compared to controls, and correlated with the upregulation of Viperin (FIGS. 17A-17B).

The data demonstrate that the cell death pathway driven by ZBP1, Casp-8, and GSDMC was also activated in IBD patients.

### IFN-λ Drives Pyroptosis of IECs.

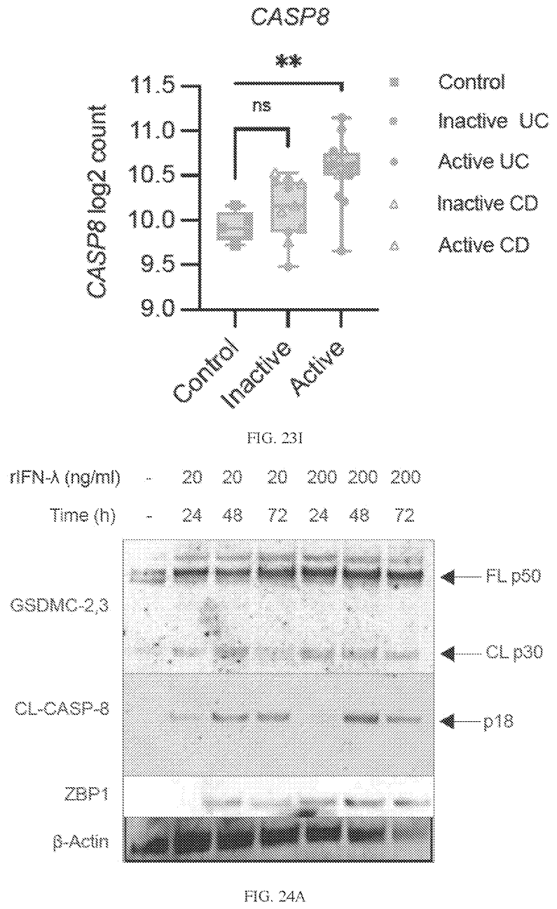

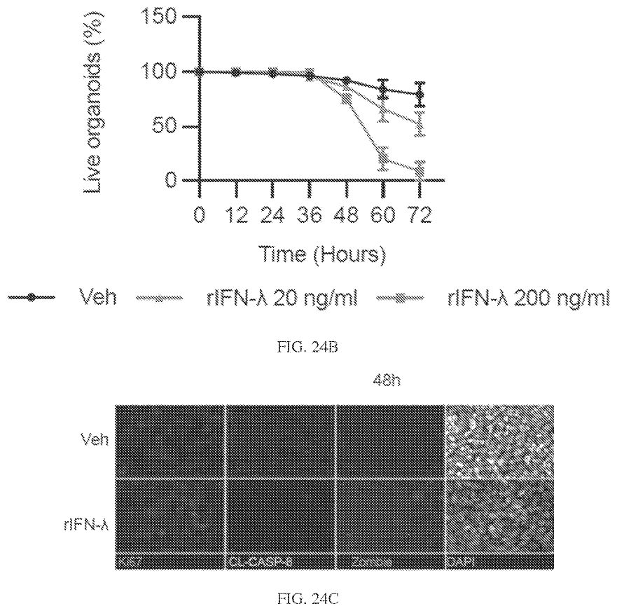

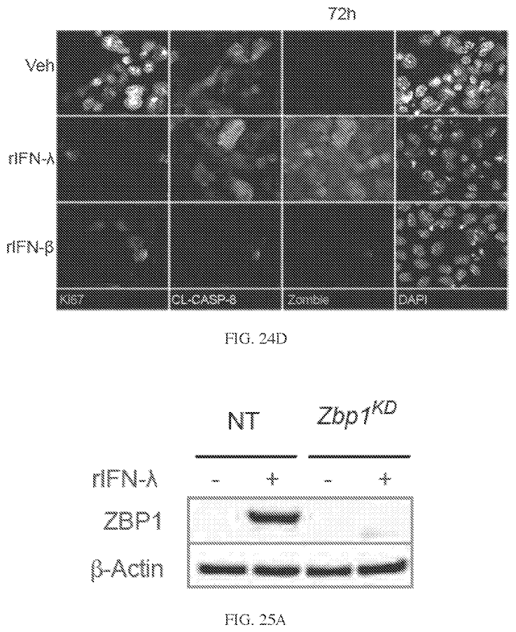

To directly assess the role of the signaling cascade initiated by IFN-λ in driving epithelial cell death intestinal organoids were used. Mouse and human small intestinal spheroids seeded in the presence of rIFN-λ died between 48 and 72 hours from treatment (FIG. 6A, 11A). Dying cells assumed typical changes associated with pyroptosis including swelling and sudden disruption of the plasma membrane and release of nuclear DNA (FIG. 6A). By using spheroids derived from WT or Stat1^{-/-} mice, it was also confirmed that gene transcription induced by IFN-λ was necessary to induce cell death (FIG. 6B). Similar to what was observed in vivo, it was determined that rIFN-λ administration to spheroids profoundly diminished the level of Lgr5 expression, suggesting a defect in the maintenance and/or proliferation of ISCs (FIG. 11B). In agreement with a reduced number of ISCs, cell proliferation and organoid growth were also found to significantly decreased in IFN-λ-treated mouse-, as well as human-, spheroids (FIGS. 6D, 611C). In keeping with the capacity of IFN-λ to activate a ZBP1/Casp-8/GSDMC axis, spheroids grown for 6 days and then administered rIFN-λ showed ZBP1 upregulation, as well as cleavage of GSDMC-2/-3 and Casp-8 (FIG. 6E). Both the activation of the ZBP1/Casp-8/GSDMC axis as well as the induction of cell death showed dose dependence (FIG. 24A-26B).

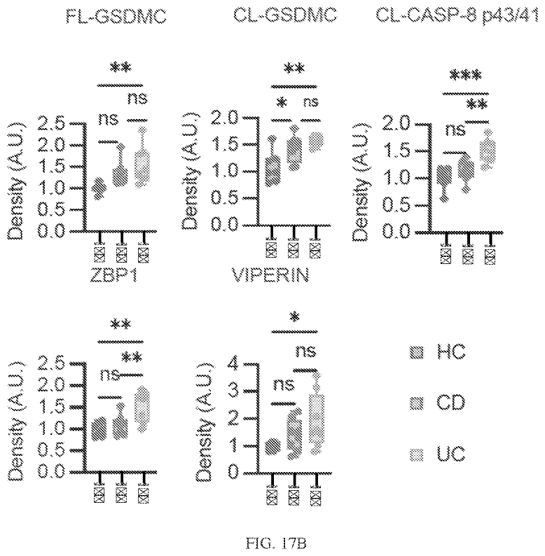

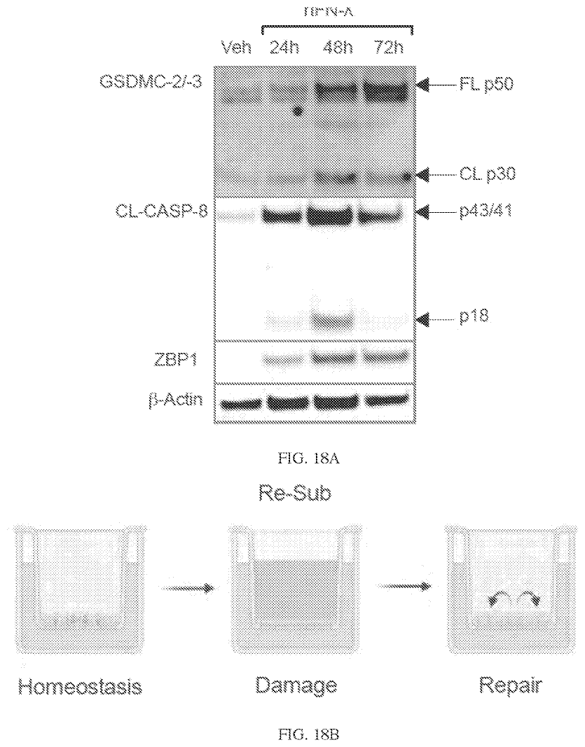

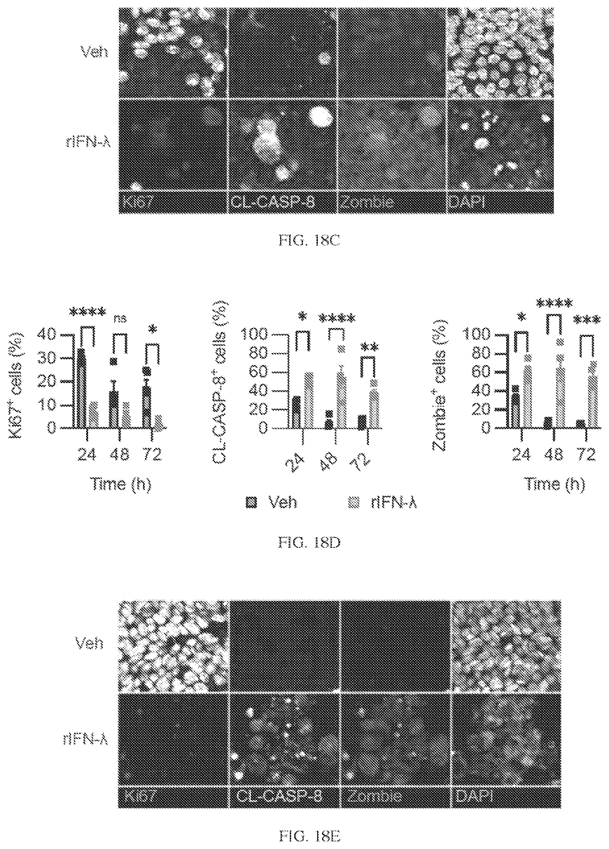

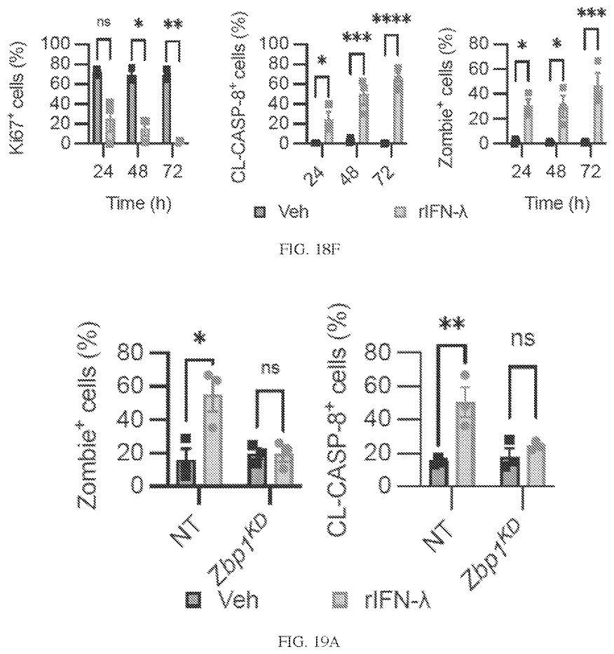

To define the activity of IFN-λ during injury and repair an air-liquid interface (ALI) organoid culture that can mimic the cycles of damage and repair that occur in IBD patients (30) was implemented (FIG. 18B). This culture system leads to the differentiation of a self-organizing monolayer that mimics homeostasis. This monolayer can then be re-submerged in medium, to induce hypoxic stress and cause damage responses, and re-exposed to air to induce epithelial regeneration responses (30).

When the homeostatic epithelial monolayer was treated with IFN-λ, it was determined that IFN-λ alone was not able to induce cell death, activate caspases, or inhibit proliferation (FIG. 24C). Conversely, when the organoids were treated concomitantly with re-submersion to mimic a damage, it was determined that rIFN-λ potentiated the damaging effect of re-submersion by increasing Casp-8 cleavage and cell death (FIGS. 18C, 18D). Moreover, while vehicle-treated organoids upregulated Ki67 and started mounting a reparative response 24 hours after re-submersion, rIFN-λ-treated organoids did not (FIGS. 18C, 18D). Importantly, treatment with type I IFNs (rIFN-0) did not potentiate the damaging effect of re-submersion, or induce activation of caspases, confirming that this pathway is preferentially stimulated by IFN-λ in IECs (FIG. 24D). Further, when cells are grown at ALI, IFN-λ stimulation alone is not sufficient to induce cell death, but that a combination of a damaging stimulus and IFN-λ signaling is necessary to induce IEC death. This was consistent with the absence of phenotypic differences at homeostasis between WT mice and Ifnlr1^{-/-} mice, in the presence or absence of rIFN-λ.

It was determined, in vivo, IFN-λ reduces the expression of proliferative transcriptional programs and reduces the number of proliferating epithelial cells. Thus, in vitro assays were performed to determine whether IFN-λ exerted similar effects when IECs were treated concomitantly with re-exposure to air, which mimics the onset of a reparative response. It was discovered that IFN-λ blunted proliferation by decreasing the proportion of Ki67⁺ proliferating cells, and, at the same time, causing caspase activation and cell death in reparatory monolayers (FIG. 18E, 18F).

Overall, this demonstrates that IFN-λ acts directly on IECs to induce cell death and to inhibit repair after epithelial cell damage.

### The ZBP-1/Casp-8/GSDMC Axis Mediates IEC Pyroptosis in Response to Damage and IFN-Encounter.

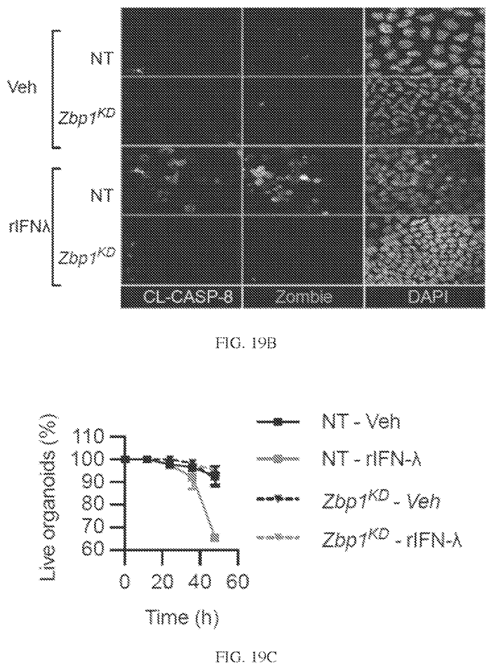

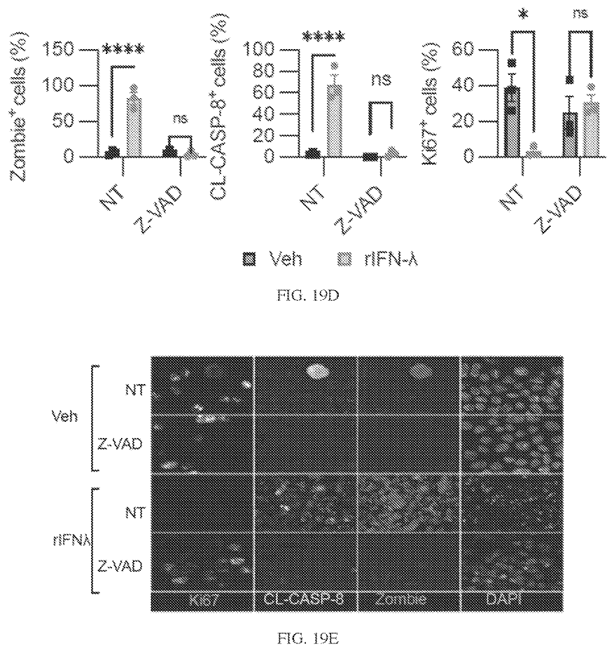

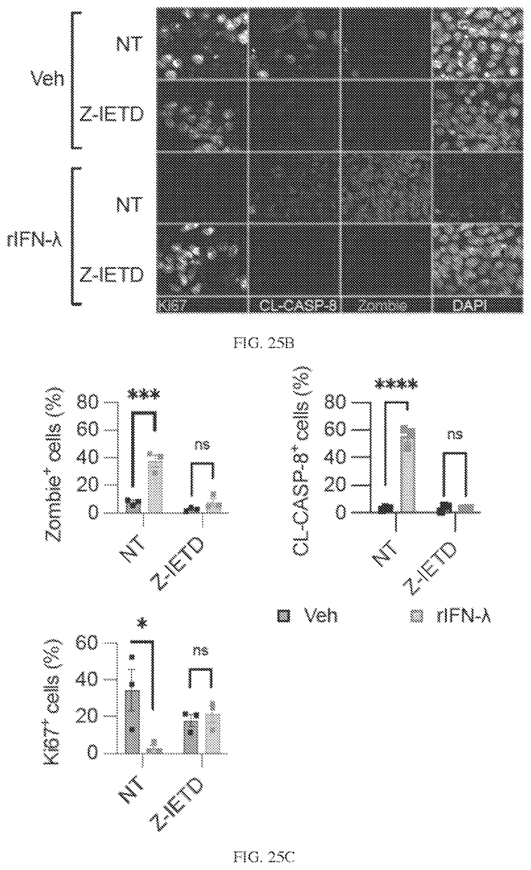

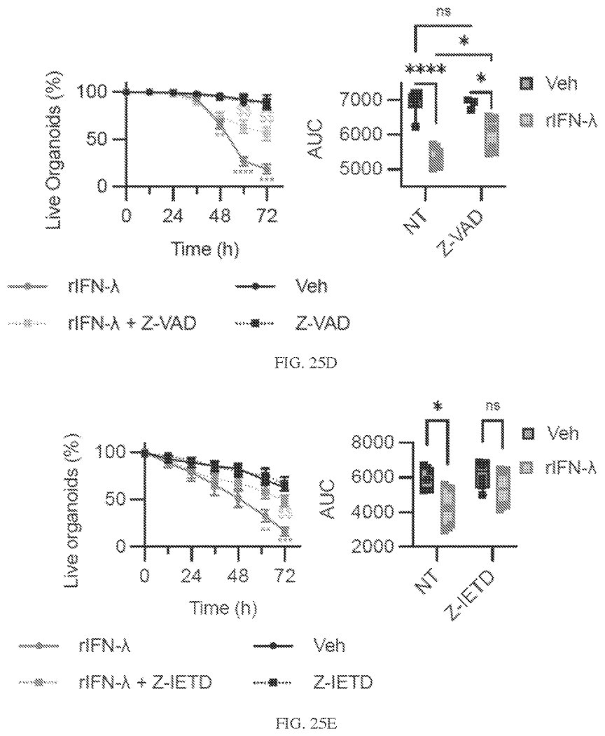

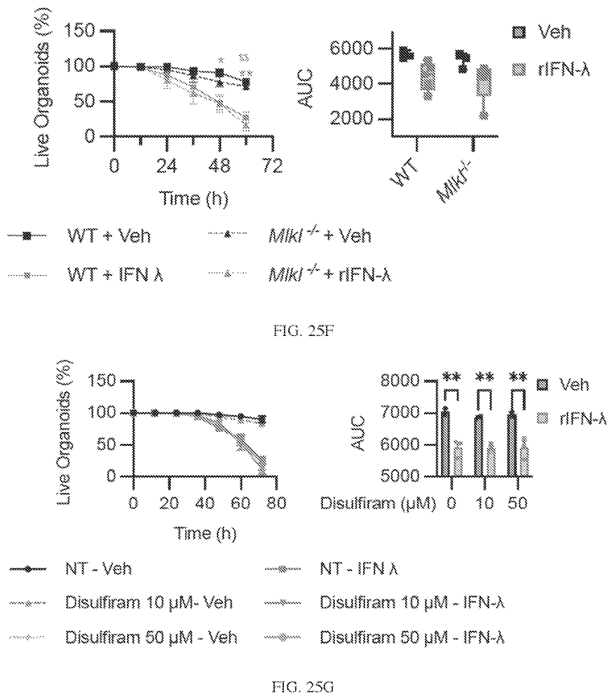

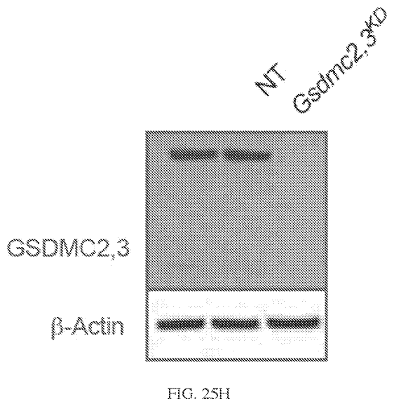

Next, the ZBP-1/Casp-8/GSDMC axis was assessed to determine if it is required to induce cell death in response to IFN-λ. Initially, the capacity of IFN-λ to induce cell death following damage induced by re-submersion of the homeostatic monolayer was tested. It was determined that organoids in which Zbp1 was knocked out using CRISPR-Cas9 (Zbp1^{KD}, FIG. 25A), cell death and Casp-8 cleavage were significantly decreased (FIGS. 19A, 19B). Similarly, Zbp1^{KD} spheroids exposed to IFN-λ were protected from cell death (FIG. 19C). Inhibition of caspase activity with the pan-caspase inhibitor Z-VAD-FMK rescued cell death, blocked Casp-8 activation, and rescued proliferation both during re-submersion induced damage (FIG. 19D, 19E) and during the recovery response triggered by re-exposure to air (FIG. 19F, 19G). Similar results were obtained with the Casp-8 specific inhibitor, Z-IETD-FMK (FIGS. 25B, 25C). Caspase inhibition rescued IFN-λ-dependent cell death also when spheroids exposed to IFN-λ were treated with the pan-caspase inhibitor Z-VAD-FMK, or the Casp-8 inhibitor, Z-IETD-FMK (FIGS. 25D, 25E). Of note, the absence of MLKL, the executor of necroptosis, or the inhibition of GSDMD oligomerization and pore formation with Disulfiram, did not rescue IFN-λ-dependent cell death (FIGS. 25F, 25G).

Finally, to directly assess the involvement of GSDMC in this process, Gsdmc-2 and Gsdmc-3 were knocked out with CRISPR/Cas9 in small intestine organoids (Gsdmc2,3^{KD}, FIG. 25H). It was discovered that upon exposure to rIFN-λ, survival of organoids that do not express Gsdmc2, 3 was significantly increased compared to controls in condition of re-submersion induced damage (FIGS. 19H, 19I), while Casp-8 activity was preserved (FIGS. 19H, 19I). Similarly, Gsdmc2,3^{KD} spheroids were protected from cell death upon exposure to IFN-λ (FIG. 19J).

Overall, these data demonstrate that IFN-λ initiates a ZBP-1/Casp-8/GSDMC axis that drives pyroptosis in IECs.

### DISCUSSION

In this work, it was revealed that IFN-λ restrains the regeneration of the epithelial barrier either during colitis or secondary to ionizing radiation toxicity. It was determined that the capacity of IFN-λ to initiate a previously overlooked molecular cascade in IECs that allows the induction of ZBP1, the activation of Casp-8, and the induction and cleavage of GSDMCs.

Components of these pathways were also activated in IBD patients. In mice, induction of epithelial cell death via the ZBP1-GSDMC axis reduced the number of ISCs and dampens the proliferation of IECs, thus affecting the re-epithelization of the injured intestine. Finally, IFN-λ, but not type I IFNs, were the major drivers of the delayed repair in vivo in mouse models of intestinal mucosal damage that follows colitis or irradiation, opening new ways of therapeutic intervention for individuals that encounter tissue damage such as IBD patients or subjects exposed to radiation therapies.

The capacity of IFN-λ to dampen lung epithelial cell proliferation and to drive their apoptosis was previously described (18, 19). Similar anti-proliferative and pro-apoptotic transcriptional programs were found in the lung of patients with severe-to-critical COVID-19 (20). Of note, the new data reveal the absence of expression of GSMDC in the lung at the protein or mRNA levels. These findings are in agreement with the previous RNAseq data that did not identify Gsdmc2,3 as genes upregulated in the lung epithelial cell of mice exposed to virus ligands that induce IFN-λ production (18). The new findings in the gut show that decreased proliferation, assessed at transcriptional and cellular levels, was due to augmented pyroptotic cell death. How IFN-λ instructs epithelial cells of different organs to initiate distinct cell death pathways (i.e.: apoptosis in the lungs and pyroptosis in the gut) remains an open question that will require further investigation.

Another interesting observation was that exogenous administration of IFN-λ does not induce caspase or GSDMC activation in vivo in the absence of tissue damage, although it induces ZBP1 upregulation at the transcriptional as well as the protein level. ZBP1 is a Z-nucleic acid binding protein and is a key node in the formation of a multiprotein complex that governs the cell fate (45). ZBP1 can interact directly or indirectly with proteins that regulate cell death and drive the activation of apoptotic caspases, the necroptosis effector MLKL, or pyroptosis effectors Casp-1, 11 and GSDMD (36, 46, 47). So far, GSDMC was not associated with ZBP1. The data highlight the existence of a ZBP1-GSDMC axis that appears to be the preferential pathway of cell death active during intestinal epithelial damage and restitution cycles. Upregulation of ZBP1 alone is not sufficient to trigger cell death, which is consistent with the inability to detect toxic effects of IFN-λ in the absence of inflammation or tissue damage, or in vitro in organoids grown at ALI that model a homeostatic monolayer. Conversely, ZBP1 can be activated both by binding microbial-derived nucleic acids (47, 48) or by binding host-derived Z-DNA following oxidative damage of the mitochondria (49), as well as dsRNA, derived from ISG transcripts or endogenous retroelements that can fold in the form of Z-RNA (50). It is, thus, possible that in vivo, under tissue-damaging conditions, either microbiota-, or host-derived nucleic acids become available to induce the activation of ZBP1.

In keeping with these observations, it was also found that the ISC compartment was not altered under homeostatic conditions in Vil^{CRE}Ifnlr1^{fl/fl} mice compared to WT mice, suggesting that the basal level of IFNs present during homeostasis (51) does not affect the homeostatic turnover of gut epithelial cells. Intriguingly, it has been recently shown that IFN-λ-dependent responses at homeostasis are restricted to pockets of mature enterocytes in the small intestine and in the colon (51). In contrast, when mice are injected with rIFN-λ, or infected with murine rotavirus, a type of virus potently controlled by IFN-λ (52), responses to IFN-λ broadly distribute along the epithelial layer. These data, together with the new findings, suggest that the compartmentalization of IFN-λ signaling at homeostasis preserves the functions of ISCs and the normal turnover of gut epithelial cells.

In organoids grown in ALI, both in the presence of hypoxic epithelial damage and in conditions of simulated repair, IFN-λ treatment increases cell death and inhibits proliferation. Importantly, blockade of caspases and genetic deletion of ZBP1 or GSDMCs not only rescued cell death during both damage and repair, but also rescued the loss of proliferating cells. This indicates that the ZBP1/Casp-8/GSDMC axis is active both during damage and tissue repair, and in both cases, it delays mucosal repair. It is also important to note that only blockade of GSDMCs, but not inhibition of necroptosis or GSDMD activation, prevents the death of IECs. These data highlight the essential role played by GSDMCs in driving pyroptosis in the gut, compared to other executors of cell death.

In contrast to organoids grown at ALI, the administration of IFN-λ to murine- or human-intestinal spheroids, activates the ZBP1-GSDMC axis and drives cell death, even in the absence of an external signal.

The models of intestinal damage either of the colon, in the DSS-colitis model, or of the small intestine, in the radiation model, highlight the centrality of IFN-λ and its capacity to delay epithelial cell regeneration. Two previous studies suggested that IFN-λ may play an opposite role and favor tissue restitution during colitis (12, 13). Nevertheless, both studies were performed by inducing colitis in total Ifnlr1^{-/-} mice and thus they suffer the confounding activity of IFN-λ on neutrophils. The absence of IFN-λ signaling in neutrophils potentiates tissue damage (9), making it hard to compare the tissue restitution phase with WT mice that start from a different level of damage. Indeed, IFNs were administered or blocked in the colitis model after the peak of the inflammatory phase. Alternatively, the mice used were deficient for the IFNLR only in epithelial cells in which the inflammatory phase of DSS-induced colitis remains unaffected (9). Total knock-out mice were solely used in the radiation model in which damage and/or inflammation are not driven by neutrophils but by ionizing radiation. The compartmentalized activity of IFN-λ in different cell types appears to be, thus, a key feature of this group of IFNs.

Overall, the data unveiled a new axis between IFN-λ, ZBP1, and GSDMC that governs mucosal repair in the gut and opens new perspectives to future therapeutic interventions.

### MATERIALS AND METHODS

Mouse strains. C57BL/6J (Jax 00664) (wild-type), Ifnar1^{-/-} (Jax 028288), MRP8^{CRE} recombinase (Jax 021614), and Vil^{CRE} recombinase (Jax 004586) mice were purchased from Jackson Labs. C57BL/6 IL-28R (Ifnlr1^{-/-}) mice were provided by Bristol-Myers Squibb. Cells from the intestine of B6.129S(Cg)-Stat1tm1D1v (Stat1^{-/-}, JAX 012606) were kindly provided by Dr. S.B. Snapper. The mutant mouse line Ifnlr1^{tm1a(EUCOMM)Wtsi} was provided by the Wellcome Trust Sanger Institute Mouse Genetics Project (Sanger MGP) and its funders (funding and associated primary phenotypic Information, sanger.ac.uk/mouseportal). Mice constitutively expressing Cas9 (Gt(ROSA)26Sortrmi 1(CAG-cas9*,-EGFP)^{Fezh/f}; JAX stock no. 024858) were kindly donated by Dr. B. Malissen. Mice were housed under specific pathogen-free conditions at Boston Children's Hospital, and all the procedures were approved under the Institutional Animal Care and Use Committee (IACUC) and operated under the supervision of the department of Animal Resources at Children's Hospital (ARCH).

Reagents and antibodies. To treat murine organoids in vitro and for in vivo administration, recombinant mouse IFNλ-2 (rIFN-λ) attached to polyethylene glycol (provided by Bristol-Myers Squibb), mouse recombinant IFN-β (12401-1; PBL interferonsource), anti-IFN-λ2-3 (MAB17892; R&D systems) and anti-IFNAR1 (BE00241; BioXCell), the pan-caspase inhibitor Z-VAD-FMK (HY-16658B; MedChem Express), EdU (NE08701; Carbosynth) were used. To treat human organoids in vitro, recombinant human IFNλ-2 (300-02K; Peprotech) was used. The following antibodies and reagents were used for immunoblotting or immunofluorescence: 0-Actin (Mouse monoclonal; A5441; Lot #127M4866V; or A3854; Lot #0000141518; AC-15 clone; Sigma-Aldrich), Rsad2 (Mouse monoclonal custom made; BioLegend) Zbp1 (Mouse monoclonal; AG-20B-0010-c100; Lot #A28231605; AdipoGen), gasdermin C (Rabbit monoclonal; 229896; Lot #GR3317481-6; abeam or Rabbit monoclonal; 225635; Lot #GR3317480-2 abeam or Rabbit polyclonal; #MBS8242472; Lot #CP1D10A; MyBiosource), gasdermin D (Rabbit polyclonal; 20770-1-AP; Proteintech), Anti-Flag-HRP (Mouse monoclonal; clone M2; A8592; Lot #SLBV3799; Sigma), Caspase 8 (D35G2; 4790; Lot #2; CST), cleaved caspase 8 (Rabbit monoclonal; Asp387; D5B2; 8592; Lot #4; CST). Ki67 (Rat monoclonal; SolA15 clone; 14-5698-82; Lot #2496198; Invitrogen), Zombie Red™ Fixable Viability Kit (423109; Lot #B337268; Biolegend), Zombie NIR™ Fixable Viability Kit (423105; Lot #B350797; Biolegend), DAPI (MBD0015; Lot #0000128033; Sigma). The plasmids for sgRNA cloning and lentiviral particles production were kindly provided by S. Roulland: pMCB320 (Addgene #89359); psPAX2 (Addgene #12260) and pMD2.G (Addgene #12259).

DSS-colitis induction. To induce colitis, mice were given 2.5% (w/v) dextran sulfate sodium (DSS, Affymetrix) in drinking water for 7 days and were then administered water for 7 days. Where indicated in the figure legends, mice received daily intraperitoneal injections of 50 μg kg⁻¹ day⁻¹ rIFN-λ or rIFN-0, and, to deplete endogenous IFN-λ or to block type I IFN signaling, mice received daily intraperitoneal injections of 12.5 μg kg⁻¹ day⁻¹ of anti-IFN-λ2-3 or anti-IFNAR1 antibody respectively. Body weight, stool consistency and the presence of blood in the stool were monitored daily. Weight change was calculated as percentage of initial weight.

Partial body irradiation. Mice were sedated with a mix of ketamine (100 mg/ml) and xylazine (20 mg/ml) intraperitoneally. Mice then received gamma irradiation in a Best Theratronics Gammacell 40 Cesium 137-based irradiator with lead shielding of the head, thorax, and upper extremities to prevent bone marrow failure. In one sitting mice received either 11 Grey of gamma irradiation to assess tissue restitution in small intestinal crypts or 14 Grey of gamma irradiation for survival experiments. To assess proliferation, mice were intraperitoneally administered a 100 mg/kg dose of EdU in saline, final volume 500 μl.

Histological Analysis. For morphologic analysis of irradiation experiments, fragments of the small intestine were fixed overnight in 4% paraformaldehyde (PFA) and embedded in paraffin for sectioning. Intestinal sections were stained with hematoxylin and eosin. Images were acquired with an EVOS M7000 (Thermo Fisher Scientific). Intact crypts were counted in 3 sections per animal blindly with ImageJ software. Proliferation was assessed by measuring the number of EdU⁺ cells per intestinal crypt. A minimum of 10 crypts per section and 3 sections per mouse were evaluated. For Immunofluorescence analysis, paraffin sections were deparaffinized with sequential washes in xylene and ethanol. Deparaffinized sections were then stained for EdU incorporation. All quantifications were executed in a blinded fashion. For histology of DSS-colitis experiments, colons were flushed with PBS, flattened and rolled into a ‘Swiss roll’. Colon rolls were fixed in 10% formalin (Fisher Scientific), dehydrated in 70% Ethanol and embedded in paraffin. Paraffin sections were stained with hematoxylin and eosin and histological features were evaluated. Histological scoring was performed in a blinded fashion by assignment of a score of 1-5 to segments of the colon roll (1, presence of leukocyte infiltrate, loss of goblet cells; 2, bottom third of the crypt compromised; 3, two thirds of the crypt compromised; 4, complete crypt architecture loss; 5, complete crypt loss and lesion of the epithelial layer). Each segment was then measured with ImageJ software, and the final histological score of each sample was obtained by ‘weighting’ the score of each segment against the length of the segment and divided by the total length of the colon roll.

Immunofluorescence. For ex-vivo experiments, paraffin sections were deparaffinized with sequential washes in xylene and ethanol. For EdU incorporation staining, deparaffinized slides or organoids were fixed in 10% formalin and were stained for 30 minutes with 2 mM Sulfo-Cyanine5-azide (Lumiprobe) in the presence of 1 mM CuSO4 and 2 mg/ml Sodium ascorbate in PBS. After EdU staining, slides or organoids were stained with DAPI (Sigma) to detect nuclei and mounted with ProLongGold antifade reagent (Thermo Fisher Scientific). For ALI experiments, organoids were pulsed for 30 minutes with Zombie dye at 1:500 in culture medium then fixed using 3.7% formaldehyde in PBS for 15 minutes. Organoids were permeabilized using 0.5% Triton X-100 in PBS, saturated with 3% BSA in PBS then stained with Ki67 and CL-Casp-8 antibodies and DAPI. Organoids were mounted with Fluoromount G (Invitrogen). Images were acquired using LSM 888 confocal microscope (Zeiss).

Intestinal epithelial and lamina propria cell isolation. Small intestines were longitudinally cut and rinsed in PBS. Mucus was washed away by incubation with 1 mM dithiothreitol (DTT) at 4 °C for 5 minutes. The tissue was moved to 10 mM EDTA, 1% fetal bovine serum (FBS), 1% sucrose at 37 °C for 5 minutes. Samples were vortexed and small intestine fragments were moved to a new tube with 10 mM EDTA, 1% FBS, 1% sucrose at 37 °C for 10 minutes. Samples were vortexed. The supernatant was filtered through a 70 μm strainer and kept on ice. Small intestine fragments were moved to a new tube with 10 mM EDTA, 1% FBS, 1% sucrose at 37 °C for 10 minutes. Samples were vortexed and the supernatant was combined with the previous fraction. The isolated crypts were resuspended in Trizol for RNA extraction and in RIPA Buffer with protease and phosphatase inhibitors for Western Blot analysis.

Colons were opened longitudinally and washed with PBS to remove fecal contents. IEC were extracted by incubating them in dissociation buffer (Hanks' Balanced Salt Solution (HBSS) supplemented with 2.5 mM EDTA, 1 mM DTT, and 5% FCS) at 37 °C, 250 rpm for 30 minutes). IEC were collected by centrifugation of dissociated cells that passed through 40 m cell strainers, while LP cells were further obtained from the retained colon pieces. Remaining intestine pieces were then washed with PBS, cut into small pieces, and incubated in digestion buffer (RPMI 1,640 medium supplemented with 10% FCS, 0.5 mg/ml collagenase I, 0.5 mg/ml collagenase IV, and 40 g/ml DNase I) at 37 °C, 250 rpm for 30 minutes. Digested tissues were homogenized in 40 m cell strainers, washed with PBS and centrifuged at 900×g for 5 minutes.

Measure of gene expression in organs, small intestine crypts and organoids. Samples were either collected in Trizol (Thermo Scientific) and RNA was isolated using phenol-chloroform extraction or for total tissue RNA extraction, 1 cm tissue pieces were homogenized in RNA lysis buffer (Bio-rad) and RNA was extracted using Aurum Total RNA mini kit (Bio-rad). For analysis of Gsdmc expression from different tissues (FIG. 22B) RNA was quantified by NanoDrop and 500 ng of RNA was reverse transcribed using iScript cDNA synthesis kit (Biorad). Real-time PCR was performed using iQ SYBR Green Supermix reagent (Bio-Rad). Mouse GSDMC qPCR primers were designed to detect all four murine GSDMC paralogs (Forward: GGAAGAGATCTGAGGCCTG (SEQ ID NO: 3), Reverse: CACTTCAGGCTCTGGAACAG (SEQ ID NO: 4)).

For all other targets purified RNA was analyzed for gene expression by qPCR on a CFX384 real-time cycler (Bio-rad) using Power SYBR™ Green RNA-to-C_{T}™ 1-Step Kit (Thermo Scientific, 4389986) and pre-designed KiCqStart SYBR Green Primers (MilliporeSigma) or IDT PrimeTime Predesigned qPCR Assays.

RNA sequencing. For targeted transcriptome sequencing, RNA (15 ng) isolated from small intestinal crypts was retro-transcribed to cDNA using SuperScript VILO cDNA Synthesis Kit (11754-05; Invitrogen). Barcoded libraries were prepared using the Ion AmpliSeq Transcriptome Mouse Gene Expression Panel, Chef-Ready Kit (A36412; Thermo Scientific) as per the manufacturer's protocol. Sequencing, read alignment, de-multiplexing, quality control and normalization was performed using an Ion S5 system (A27212; Ion Torrent, Torrent Suite software v5.12.1). The generated count matrices were analyzed using custom scripts in R (v 4.1.1). Differential Expression of Gene analysis was performed using the R package DEseq2 (v 1.34) with shrinkage of log 2 fold changes. Volcano plots were created using the R package EnhancedVolcano (v 1.12). The differentially expressed genes with an adjusted p-value lesser that 0.1 and a log 2 fold change greater than 1.5 were selected for downstream analysis. Functional enrichment analysis in Gene Ontology was performed using the R package ClusterProfiler (v 4.2) with the Biological Process terms and Benjamin-Hochberg multi-test correction with 5% of FDR threshold. Geneset Enrichment Analysis (GSEA) of hallmarks was performed using the R package fgsea (v 1.20) using the hallmark genesets (v 7.4) from the Broad Institute MSigDB or custom genesets. Leading edges of the different selected genesets were selected to build heatmaps of their expression in the different conditions and samples. The R package ComplexHeatmap (v 2.10) was used to plot the heatmaps. CIBERSORTx (28) was used to estimate the abundances of epithelial cell types using bulk gene expression data as an input and scRNAseq signature matrices from single-cell RNA sequencing data to provide the reference gene expression profiles of pure cell populations. The scRNAseq signature matrix used to deconvolute RNAseq dataset from small intestine crypts was taken from (29). Code is available on GitHUb: github.com/CIML-bioinformatic/ABlab_IFNLGutRestitution

Western blot. Western blot was performed with standard molecular biology techniques. Blots were probed for: β-Actin, Rsad2, Zbp1, gasdermin C, gasdermin D, caspase 8, and cleaved caspase 8.

RNAseq on IBD patient's biopsies. The IBD biobank was generated starting with biopsy samples collected from patients suffering from CD or UC and diagnosed as clinically quiescent or in an active phase of the disease with various degrees. Controls were taken from non-inflammatory healthy portions of the colon. This investigation was registered under ClinicalTrials.gov Identifier: NCT02304666. A detailed description of the biobank as well as the RNAseq studies, are described elsewhere (44). All raw and processed sequencing data generated in this study are accessible on the NCBI Gene Expression Omnibus (GEO) under the meta-series GSE174159 (ncbi.nlm.nih.gov/geo/query/acc.cgi?acc=GSE174159).

Samples were divided in three groups according to the disease status of the patient: control, inactive and active. The mean of genes for the tested hallmarks were computed by mean of their expression across the samples of each group. Linear regression analyses were performed using the geom_smooth function of the R package ggplot (v 3.3.5) with the “lm” method. Correlation analyses were performed by a Spearman test using the cor.test function of the R base package stats.

Code is available on GitHUb: github.com/CIML-bioinformatic/ABlab_IFNLGutRestitution Bulk RNA-seq sequencing data was downloaded from NCBI GEO for the RISK (ncbi.nlm.nih.gov/geo/query/acc.cgi?acc=GSE57945; TPM-normalized counts matrix) and PROTECT (ncbi.nlm.nih.gov/geo/query/acc.cgi?acc=GSE109142; RPKM-normalized count matrix) cohorts. For the RISK data, samples with “undetermined” histopathology data were excluded from the analysis, and IBD samples labeled as macroscopically or microscopically inflammation were categorized as “Active” with the rest as “Inactive”. For the PROTECT cohort, samples lacking histology scores were excluded from the analysis and all other IBD samples were categorized as “Active” if they had a Histology Severity Score (for chronic and active acute neutrophil inflammation) >1 and “Inactive” if they had a Histology Severity Score of 0-1 (54). Group comparisons between healthy controls, inactive and active IBD were performed using non-parametric t-testing (Wilcoxon test) and p values reported.

Human biopsies. Colonic tissue from pediatric (age >6, <21 years) subjects were collected after providing written informed consent. Samples were collected peri-procedure (colectomy/colonoscopy) at Boston Children's Hospital under Institutional Review Board protocol IRB-P00000529. Biopsies were collected in 500 mL RPMI 1640 medium (ThermoFisher), 50 mL FBS plus 5 mL of: Pen/Strep (ThermoFisher), NEAA, Sodium pyruvate, Glutamax, and HEPES (Gibco). Samples were cryopreserved in freezing media (10% dimethyl sulfoxide (DMSO) (Sigma) and 90% FBS (Gibco). Samples were transferred to liquid nitrogen for long-term storage.

Molecular cloning and plasmid transfection. cDNA coding for the full-length human GSDMC (hGSDMC FL) was amplified from HaCaT cells and cloned into a cFlag pcDNA 3 vector under control of CMV promoter (Addgene #20011). The pCDH-CuO-MCS-EF1a-GFP+Puro plasmid containing the N-terminus of human GSDMC (hGSDMC NT) was kindly provided by QuenchBio. Mouse GSDMD N-term-pRetroX TetONE-eGFP plasmid was kindly provided by Dr. Petr Broz (55). FL or NT hGSDMC was cloned into pRetroX TetONE-eGFP using standard cloning procedures and verified by Sanger Sequencing. This yielded a doxycycline inducible expression of hGSDMC FL or NT and constitutive expression of GFP. To express human GSDMC FL or NT, HEK293T cells were seeded into 96-well plate (50,000 cells per well) at the day of transfection and subsequently transfected with 100 or 200 ng of plasmid DNA in GeneJuice transfection reagent (Millipore Sigma) following the manufacturer's guidelines. Twenty-four hours post-transfection LDH release and cell viability were determined using CytoTox 96® Non-Radioactive Cytotoxicity Assay and CellTiter-Glo® Luminescent Cell Viability Assay detection kits (both Promega), respectively. Time course experiments for cell death induction were performed using SYTOX Orange and the Biotek Cytation5.

Organoid culture. Mouse intestinal spheroids were derived and maintained as previously described (56). Briefly, 1 cm long segments of the small intestine were incubated in 10 ml of 2 mM EDTA for 60 minutes at 4 °C under rotation. Following the incubation period, the tubes were vigorously shaken, and the supernatant was passed through a 70 m strainer to collect small intestinal crypts and exclude villi fragments. The crypt compartment was collected by centrifugation, washed with advanced DMEM/F12 media (Thermo Fisher Scientific), resuspended in cold Matrigel (Corning) and plated in 40 μl domes with 50% L-WRN (Wnt3, R-spondin, Noggin) supplemented medium (Organoid Growth Medium). Human organoids derived from healthy patients' duodenal biopsies were kindly provided by Dr. Jay Thiagarajah. Duodenal biopsy samples were obtained from routine diagnostic endoscopy under Boston Children's Hospital IRB protocol P00027983 and cultured with methods modified from (57). Briefly, crypts were dissociated from duodenal biopsy samples obtained from age-matched (<3 years) healthy control individuals. Isolated crypts were suspended in Matrigel and plated in 50 μL domes with 50% L-WRN supplemented media.

For maintenance, organoids were liberated from the extracellular matrix by incubating in cell recovery solution (Corning) at 4 °C for 60 minutes, then a single cell preparation was obtained by incubating in TrypLE express (Thermo Fisher Scientific) at 37 °C for 10 minutes. Single cells were then re-plated in Matrigel with 50% L-WRN supplemented media and 10 μM Rock inhibitor Y-27632. For western blot experiments, organoids were plated for 6 days. Organoids were then treated as indicated in the figure legends by adding cytokines and reagents to the medium overlaying the organoids. At the indicated timepoints, organoids were liberated from the extracellular matrix and lysed in RIPA buffer with protease and phosphatase inhibitors. For microscopy, freshly isolated crypts were seeded in 10 L of extracellular matrix in μ-Slide 15 Well 3D (Ibidi) and overlayed with 45 l of 50% L-WRN conditioned medium supplemented with 10 μM Rock inhibitor Y-27632 for 24 hours then incubated with 50% L-WRN conditioned medium for another 24 hours. Following incubation, organoids were treated according to the figure legend in the presence of 1 μg/ml Propidium Iodide (PI) (Sigma), and incubated in the video microscope “Observer Z.1 Zeiss with Hamamatsu ORCA Flash 4.0 LT”, equipped with a temperature controlled CO₂ chamber. Wells were scanned every 12 hours and mosaic brightfield and fluorescence images were taken. Organoids were identified by ImageJ and were followed over time for PI incorporation as hallmark of cell death. Percent of live organoids was expressed as percent of organoids that never incorporated PI. Where indicated in the figure legends, organoids viability was measured with CellTiter-Blue (Promega) according to the manufacturer's instruction. Percent of live organoids is expressed based on relative CellTiter-Blue signal compared to untreated organoids.

For experiments with 2D organoids in Air-Liquid Interface (ALI), a previously described protocol was followed (30). Briefly, cultured mouse small intestinal organoids were dissociated in single cells and seeded on polycarbonate transwells, with 0.4 m pores (CORNING). Initially cells were seeded in the presence of 50% L-WRN media with 10 μM Rock inhibitor Y-27632 in both the lower and the upper chamber. After 7 days, the media was removed from the upper chamber to create an ALI. Cells were maintained in these conditions for 14 days to establish a homeostatic monolayer. The ALI culture was then re-submerged with 200 L 50% L-WRN medium, for 7 days and re-exposed to air for 3 days in the presence or absence of rIFN-λ, as indicated in the figure legends.

### GSDMC and ZBP1 Knock Down in Small Intestine Organoids.

GSDMC (Gsdmc2, 3^{KD}) and ZBP1 (Zbp1^{KD}) knockdown stable cell lines were produced using CrispR/Cas9 technology. gRNAs targeting the mouse GSDMC isoforms Gsdmc2, Gsdmc3 and Gsdmc4, were designed using the IDT guide design tool (Gsdmc-3-TCAGTTTCCCAACCTGATCG and Gsdmc-4-CCTGGAGTATGTGAAATAGG). gRNA targeting mouse ZBP1 (TGAGCTATGACGGACAGACG) was derived from the published mouse CRISPR Knockout Pooled Library (Brie) (58). Non-targeting gRNA (GACGGAGGCTAAGCGTCGCAA) was used as a negative control. The gRNAs were cloned into the plasmid pMCB320 (Addgene #89359). Lentivirus was packaged by co-transfecting sgRNA-containing pMCB320, with the packaging plasmids psPAX2 and pVSVG, into HEK293FT cells with Trans-IT 293T (Mirus cat. MIR2705). Lentivirus particles were harvested in cell culture media 48 and 72 hours after transfection, filtered through a 45 μm filter and concentrated with Lenti-X™ Concentrator (Takara 631232) according to the manufacturer's protocol. Organoids were transduced as previously described (59). Briefly duodenoids were removed from Matrigel by incubating at 4 °C in Cell Recovery Solution (Corning) and dissociated into single cells by incubation in TrypLE for 10 minutes at 37 °C. After centrifugation at 600×g for 5 minutes, single cells were seeded in 48 Well plate in 150 μl of Organoid Growth Media+100 μl of concentrated lentivirus particles supplemented with 4 μg/mL polybrene. Cells were spin-transduced in a pre-warmed centrifuge at 600×g for 1 hour at 32 °C, then incubated for 4 hours at 37 °C. Cells were then collected, resuspended in Matrigel, and plated in a 6-well plate. After 48 hours medium was changed and puromycin was added at 6 μg/ml to select the transduced organoids.

Quantification and Statistical Analysis. Statistical significance was assessed by: Unpaired t-test to compare two groups, One-way ANOVA with Dunnett's correction for multiple comparisons to compare 3 or more independent groups, Two-way ANOVA with Tukey's or Šidak's correction for multiple comparisons to compare two groups with two conditions. Kruskal-Wallis test with Dunn's post hoc test was used to compare 3 or more independent groups when data did not meet the normality assumption. Spearman correlation analysis was used to examine the degree of association between two continuous variables. To establish the appropriate test, normal distribution and variance similarity were assessed with the D'Agostino-Pearson omnibus normality test. Statistical analyses were two-sided and performed using Prism9 (Graphpad) software or using custom scripts in R (v 4.1.1) and details are indicated in figure legends. Throughout the paper statistical significancy is defined as follows: ns, not significant (p>0.05); *p<0.05, **p<0.01, ***p<0.001, and ****p<0.0001. For the RNA-sequencing experiment statistical significance was defined as p<0.005.

### Example 5 References

1. M. L. Meizlish, R. A. Franklin, X. Zhou, R. Medzhitov, Tissue Homeostasis and Inflammation. Annu Rev Immunol 39, 557-581 (2021).
2. S. Danese, C. Fiocchi, Ulcerative colitis. The New England journal of medicine 365, 1713-1725 (2011).
3. G. Roda et al., Crohn's disease. Nat Rev Dis Primers 6, 22 (2020).
4. G. Pineton de Chambrun, L. Peyrin-Biroulet, M. Lemann, J. F. Colombel, Clinical implications of mucosal healing for the management of IBD. Nat Rev Gastroenterol Hepatol 7, 15-29 (2010).
5. E. J. Villablanca, K. Selin, C. R. H. Hedin, Mechanisms of mucosal healing: treating inflammatory bowel disease without immunosuppression?Nat Rev Gastroenterol Hepatol 19, 493-507 (2022).
6. S. V. Kotenko et al., IFN-lambdas mediate antiviral protection through a distinct class II cytokine receptor complex. Nat Immunol 4, 69-77 (2003).
7. P. Sheppard et al., IL-28, IL-29 and their class II cytokine receptor IL-28R. Nat Immunol 4, 63-68 (2003).
8. L. Prokunina-Olsson et al., A variant upstream of IFNL3 (IL28B) creating a new interferon gene IFNL4 is associated with impaired clearance of hepatitis C virus. Nat Genet 45, 164-171 (2013).
9. A. Broggi, Y. Tan, F. Granucci, I. Zanoni, IFN-lambda suppresses intestinal inflammation by non-translational regulation of neutrophil function. Nat Immunol 18, 1084-1093 (2017).
10. A. Broggi, F. Granucci, I. Zanoni, Type III interferons: Balancing tissue tolerance and resistance to pathogen invasion. J Exp Med 217, (2020).
11. I. Rauch et al., Noncanonical Effects of IRF9 in Intestinal Inflammation: More than Type I and Type III Interferons. Mol Cell Biol 35, 2332-2343 (2015).
12. C. McElrath et al., Critical role of interferons in gastrointestinal injury repair. Nat Commun 12, 2624 (2021).
13. M. T. Chiriac et al., Activation of Epithelial Signal Transducer and Activator of Transcription 1 by Interleukin 28 Controls Mucosal Healing in Mice With Colitis and Is Increased in Mucosa of Patients With Inflammatory Bowel Disease. Gastroenterology 153, 123-138 e128 (2017).
14. A. S. Henden et al., IFN-lambda therapy prevents severe gastrointestinal graft-versus-host disease. Blood 138, 722-737 (2021).
15. C. Gunther et al., Interferon Lambda Promotes Paneth Cell Death Via STAT1 Signaling in Mice and Is Increased in Inflamed Ileal Tissues of Patients With Crohn's Disease. Gastroenterology 157, 1310-1322 e1313 (2019).
16. T. Sato et al., Paneth cells constitute the niche for Lgr5 stem cells in intestinal crypts. Nature 469, 415-418 (2011).
17. M. Schmitt et al., Paneth Cells Respond to Inflammation and Contribute to Tissue Regeneration by Acquiring Stem-like Features through SCF/c-Kit Signaling. Cell reports 24, 2312-2328 e2317 (2018).
18. A. Broggi et al., Type III interferons disrupt the lung epithelial barrier upon viral recognition. Science 369, 706-712 (2020).
19. J. Major et al., Type I and III interferons disrupt lung epithelial repair during recovery from viral infection. Science 369, 712-717 (2020).
20. B. Sposito et al., The interferon landscape along the respiratory tract impacts the severity of COVID-19. Cell 184, 4953-4968 e4916 (2021).
21. T. Mahlakoiv, P. Hernandez, K. Gronke, A. Diefenbach, P. Staeheli, Leukocyte-derived IFN-alpha/beta and epithelial IFN-lambda constitute a compartmentalized mucosal defense system that restricts enteric virus infections. PLoS Pathog 11, e1004782 (2015).
22. C. K. Kim, V. W. Yang, A. B. Bialkowska, The Role of Intestinal Stem Cells in Epithelial Regeneration Following Radiation-Induced Gut Injury. Curr Stem Cell Rep 3, 320-332 (2017).
23. C. Blanpain, E. Fuchs, Stem cell plasticity. Plasticity of epithelial stem cells in tissue regeneration. Science 344, 1242281 (2014).
24. M. Quir6s, A. Nusrat, Contribution of Wound-Associated Cells and Mediators in Orchestrating Gastrointestinal Mucosal Wound Repair. Annu. Rev. Physiol. 81, 1-21 (2018).
25. S. Yui et al., YAP/TAZ-Dependent Reprogramming of Colonic Epithelium Links ECM Remodeling to Tissue Regeneration. Cell Stem Cell 22, 35-49 e37 (2018).
26. C. Metcalfe, N. M. Kljavin, R. Ybarra, F. J. de Sauvage, Lgr5⁺ stem cells are indispensable for radiation-induced intestinal regeneration. Cell Stem Cell 14, 149-159 (2014).
27. K. L. VanDussen, N. M. Sonnek, T. S. Stappenbeck, L-WRN conditioned medium for gastrointestinal epithelial stem cell culture shows replicable batch-to-batch activity levels across multiple research teams. Stem Cell Res 37, 101430 (2019).
28. A. M. Newman et al., Determining cell type abundance and expression from bulk tissues with digital cytometry. Nat Biotechnol 37, 773-782 (2019).
29. A. L. Haber et al., A single-cell survey of the small intestinal epithelium. Nature 551, 333-339 (2017).
30. Y. Wang et al., Long-Term Culture Captures Injury-Repair Cycles of Colonic Stem Cells. Cell 179, 1144-1159 e1115 (2019).
31. T. E. Ohara, M. Colonna, T. S. Stappenbeck, Adaptive differentiation promotes intestinal villus recovery. Dev Cell 57, 166-179 e166 (2022).
32. A. Ayyaz et al., Single-cell transcriptomes of the regenerating intestine reveal a revival stem cell. Nature 569, 121-125 (2019).
33. P. W. Tetteh et al., Replacement of Lost Lgr5-Positive Stem Cells through Plasticity of Their Enterocyte-Lineage Daughters. Cell Stem Cell 18, 203-213 (2016).
34. T. Kuriakose, T. D. Kanneganti, ZBP1: Innate Sensor Regulating Cell Death and Inflammation. Trends Immunol 39, 123-134 (2018).
35. S. B. Kovacs, E. A. Miao, Gasdermins: Effectors of Pyroptosis. Trends in cell biology 27, 673-684 (2017).
36. P. Broz, P. Pelegrin, F. Shao, The gasdermins, a protein family executing cell death and inflammation. Nat Rev Immunol 20, 143-157 (2020).
37. J. Hou et al., PD-L1-mediated gasdermin C expression switches apoptosis to pyroptosis in cancer cells and facilitates tumour necrosis. Nature cell biology 22, 1264-1275 (2020).
38. J. Y. Zhang et al., The metabolite alpha-KG induces GSDMC-dependent pyroptosis through death receptor 6-activated caspase-8. Cell Res 31, 980-997 (2021).
39. R. Xi et al., Up-regulation of gasdermin C in mouse small intestine is associated with lytic cell death in enterocytes in worm-induced type 2 immunity. Proc Natl Acad Sci USA 118, (2021).
40. M. Zhao et al., Epithelial STAT6 O-GlcNAcylation drives a concerted anti-helminth alarmin response dependent on tuft cell hyperplasia and Gasdermin C. Immunity 55, 1327 (2022).
41. O. Julien, J. A. Wells, Caspases and their substrates. Cell Death Differ 24, 1380-1389 (2017).
42. Y. Haberman et al., Ulcerative colitis mucosal transcriptomes reveal mitochondriopathy and personalized mechanisms underlying disease severity and treatment response. Nat Commun 10, 38 (2019).
43. Y. Haberman et al., Pediatric Crohn disease patients exhibit specific ileal transcriptome and microbiome signature. J Clin Invest 124, 3617-3633 (2014).
44. V. Millet et al., Harnessing the Vnnl pantetheinase pathway boosts short chain fatty acids production and mucosal protection in colitis. Gut, (2022).
45. D. E. Place, S. Lee, T. D. Kanneganti, PANoptosis in microbial infection. Curr Opin Microbiol 59, 42-49 (2021).
46. W. Chen, J. M. Gullett, R. E. Tweedell, T. D. Kanneganti, Innate immune inflammatory cell death: PANoptosis and PANoptosomes in host defense and disease. Eur J Immunol, e2250235 (2023).
47. H. I. Muendlein et al., ZBP1 promotes LPS-induced cell death and IL-1beta release via RHIM-mediated interactions with RIPK1. Nat Commun 12, 86 (2021).
48. T. Kuriakose, M. Zheng, G. Neale, T. D. Kanneganti, IRF1 Is a Transcriptional Regulator of ZBP1 Promoting NLRP3 Inflammasome Activation and Cell Death during Influenza Virus Infection. J Immunol 200, 1489-1495 (2018).
49. B. Szczesny et al., Mitochondrial DNA damage and subsequent activation of Z-DNA binding protein 1 links oxidative stress to inflammation in epithelial cells. Scientific reports 8, 914 (2018).
50. T. Zhang et al., ADAR1 masks the cancer immunotherapeutic promise of ZBP1-driven necroptosis. Nature 606, 594-602 (2022).
51. J. A. Van Winkle et al., Homeostatic interferon-lambda response to bacterial microbiota stimulates preemptive antiviral defense within discrete pockets of intestinal epithelium. Elife 11, (2022).
52. F. C. Walker, P. R. Sridhar, M. T. Baldridge, Differential roles of interferons in innate responses to mucosal viral infections. Trends Immunol 42, 1009-1023 (2021).
53. A. Liberzon et al., The Molecular Signatures Database (MSigDB) hallmark gene set collection. Cell Syst 1, 417-425 (2015).
54. B. Boyle et al., Histologic Correlates of Clinical and Endoscopic Severity in Children Newly Diagnosed With Ulcerative Colitis. Am J Surg Pathol 41, 1491-1498 (2017).
55. L. Sborgi et al., GSDMD membrane pore formation constitutes the mechanism of pyroptotic cell death. EMBO J 35, 1766-1778 (2016).
56. H. Miyoshi, T. S. Stappenbeck, In vitro expansion and genetic modification of gastrointestinal stem cells in spheroid culture. Nat Protoc 8, 2471-2482 (2013).
57. T. Sato et al., Long-term expansion of epithelial organoids from human colon, adenoma, adenocarcinoma, and Barrett's epithelium. Gastroenterology 141, 1762-1772 (2011).
58. J. G. Doench et al., Optimized sgRNA design to maximize activity and minimize off-target effects of CRISPR-Cas9. Nat Biotechnol 34, 184-191 (2016).
59. J. F. Van Lidth de Jeude, J. L. Vermeulen, P. S. Montenegro-Miranda, G. R. Van den Brink, J. Heijmans, A protocol for lentiviral transduction and downstream analysis of intestinal organoids. J Vis Exp, (2015).

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method for treating an inflammatory bowel disease (IBD) in a subject, the method comprising administering to the subject an effective amount of an agent that is sufficient to inhibit interferon lambda (IFNL) signaling in intestinal epithelial cells of the subject.

2. The method of claim 1, wherein the agent comprises an antibody, a small molecule, a nucleic acid, or a gene editing agent.

3. The method of claim 2, wherein the antibody binds to IFNL or interferon lambda receptor (IFNLR).

4. The method of claim 3, wherein the antibody specifically binds to IFNL or IFNLR.

5. The method of claim 3 or 4, wherein the antibody binds to IFNL or IFNLR on the cell surface of intestinal epithelial cells of the subject.

6. The method of any one of claims 3-5, wherein the antibody inhibits the activity of IFNL or IFNLR in intestinal epithelial cells of the subject.

7. The method of claim 6, wherein the antibody inhibits the activity of IFNL or IFNLR in intestinal epithelial cells of the subject by at least 10%, at least 20%, at least 30%, at least 40%, at least 50%, at least 60%, at least 70%, at least 80%, at least 90%, at least 95%, or at least 99%.

8. The method of claim 2, wherein the small molecule binds to interferon lambda receptor (IFNLR).

9. The method of claim 8, wherein the small molecule binds to IFNLR on the cell surface of intestinal epithelial cells of the subject.

10. The method of claim 8 or 9, wherein the small molecule inhibits the activity of IFNLR in intestinal epithelial cells of the subject.

11. The method of claim 10, wherein the small molecule inhibits the activity of IFNLR in intestinal epithelial cells of the subject by at least 10%, at least 20%, at least 30%, at least 40%, at least 50%, at least 60%, at least 70%, at least 80%, at least 90%, at least 95%, or at least 99%.

12. The method of claim 2, wherein the small molecule binds to a Janus kinase (JAK) protein that is bound to interferon lambda receptor (IFNLR) on the cell surface of intestinal epithelial cells of the subject.

13. The method of claim 12, wherein the JAK protein is Janus kinase 2 (JAK2).

14. The method of claim 12 or 13, wherein the small molecule does not bind to a JAK protein that is bound to interferon alpha receptor (IFNAR) on the cell surface of intestinal epithelial cells of the subject.

15. The method of any one of claims 12-14, wherein the small molecule inhibits the activity of the JAK protein in intestinal epithelial cells of the subject.

16. The method of claim 15, wherein the small molecule inhibits the activity of the JAK protein in intestinal epithelial cells of the subject by at least 10%, at least 20%, at least 30%, at least 40%, at least 50%, at least 60%, at least 70%, at least 80%, at least 90%, at least 95%, or at least 99%.

17. The method of claim 2, wherein the small molecule binds to a factor of PANoptosis signaling in intestinal epithelial cells of the subject, or Viperin (RSAD2).

18. The method of claim 17, wherein the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein.

19. The method of claim 18, wherein the caspase protein is caspase-8, caspase-3, caspase-7, or caspase-1.

20. The method of claim 18, wherein the gasdermin protein is Gasdermin C or Gasdermin D.

21. The method of claim 18, wherein the factor of PANoptosis signaling is RIPK1 and the small molecule is necrostatin.

22. The method of any one of claims 17-20, wherein the small molecule inhibits the activity of the factor of PANoptosis signaling in intestinal epithelial cells of the subject.

23. The method of claim 22, wherein the small molecule is selected from Z-VAD-FMK or Z-IETD-FMK.

24. The method of claim 23, wherein the small molecule inhibits the activity of the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

25. The method of claim 2, wherein the nucleic acid is a small interfering RNA (siRNA) or a short hairpin RNA (shRNA).

26. The method of claim 25, wherein the nucleic acid inhibits the expression of interferon lambda receptor (IFNLR), a Janus kinase (JAK) protein, Viperin (RSAD2), or a factor of PANoptosis signaling in intestinal epithelial cells of the subject.

27. The method of claim 26, wherein the JAK protein is Janus kinase 2 (JAK2).

28. The method of claim 26, wherein the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein.

29. The method of claim 28, wherein the caspase protein is caspase-8, caspase-3, caspase-7, or caspase 1.

30. The method of claim 28, wherein the gasdermin protein is Gasdermin C or Gasdermin D.

31. The method of any one of claims 25-30, wherein the nucleic acid inhibits the expression of IFNLR, the JAK protein, or the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

32. The method of claim 2, wherein the gene editing agent comprises a zinc finger nuclease (ZFN), a transcription activator-like effector nuclease (TALEN), or a clustered regularly interspaced short palindromic repeat (CRISPR)-Cas-associated nuclease (CRISPR/CAS).

33. The method of claim 32, wherein the gene editing agent comprises CRISPR/CAS and further comprises a short guide RNA (sgRNA) that is complementary to a gene encoding interferon lambda receptor (IFNLR), a Janus kinase (JAK) protein, or a factor of PANoptosis signaling in intestinal epithelial cells of the subject.

34. The method of claim 32 or 33, wherein the gene editing agent binds to and modifies a gene encoding interferon lambda receptor (IFNLR), a Janus kinase (JAK) protein, Viperin (RSAD2), or a factor of PANoptosis signaling in intestinal epithelial cells of the subject.

35. The method of claim 33 or 34, wherein the JAK protein is Janus kinase 2 (JAK2).

36. The method of claim 33 or 34, wherein the factor of PANoptosis signaling is Z-DNA-binding protein 1 (ZBP1), receptor-interacting serine/threonine-protein kinase 1 (RIPK1), receptor-interacting serine/threonine-protein kinase 3 (RIPK3), Fas associated via death domain (FADD), mixed lineage kinase domain-like pseudokinase (MLKL), dynamin-related protein 1 (Drp1), PGAM family member 5 (PGAM-5), a caspase protein, or a gasdermin protein.

37. The method of claim 36, wherein the caspase protein is caspase-8, caspase-3, caspase-7, or caspase-1.

38. The method of claim 36, wherein the gasdermin protein is Gasdermin C or Gasdermin D.

39. The method of any one of claims 34-38, wherein the gene editing agent reduces the expression of IFNLR, the JAK protein, or the factor of PANoptosis signaling in intestinal epithelial cells of the subject by at least 10%, at least 20%, at least 30%, at least 40%, at least 50%, at least 60%, at least 70%, at least 80%, at least 90%, at least 95%, or at least 99%.

40. The method of any one of claims 2-39, wherein the agent further comprises a delivery agent.

41. The method of claim 40, wherein the delivery agent is a peptide, an antibody, a liposome, or a viral particle.

42. The method of claim 41, wherein the delivery agent binds specifically to the cell surface of intestinal epithelial cells of the subject.

43. The method of any one of claims 40-42, wherein the agent and the delivery agent are covalently linked.

44. The method of claim 43, wherein the agent and the delivery agent are linked by a cleavable linker.

45. The method of claim 44, wherein the cleavable linker is a protease-sensitive linker, a pH-sensitive linker, or a glutathione-sensitive linker.

46. The method of claim 45, wherein the agent and the delivery agent are linked by a non-cleavable linker.

47. The method of any one of claims 39-46, wherein the delivery agent enhances delivery of the agent to intestinal epithelial cells of the subject, as compared to delivery of the agent to intestinal epithelial cells of the subject in the absence of the delivery agent.

48. The method of any one of claims 39-47, wherein the delivery agent enhances internalization of the agent by intestinal epithelial cells of the subject, as compared to internalization of the agent by intestinal epithelial cells of the subject in the absence of the delivery agent.

49. The method of any one of claims 1-48, wherein the method results in a reduction of inflammation in intestinal epithelial cells of the subject.

50. The method of any one of claims 1-49, wherein the method results in a reduction of cell death in intestinal epithelial cells of the subject.

51. The method of claim 50, wherein the cell death is cell death as a result of apoptosis, pyroptosis, and/or necroptosis.

52. The method of any one of claims 1-51, wherein the method results in increased proliferation of intestinal epithelial cells of the subject.

53. The method of any one of claims 1-52, wherein the method results in an enhancement of tissue repair in intestinal epithelial cells of the subject.

54. The method of any one of claims 1-53, wherein the IBD is Crohn's disease (CD).

55. The method of any one of claims 1-53, wherein the IBD is ulcerative colitis (UC).

56. The method of any one of claims 1-55, wherein the subject is a mammal.

57. The method of claim 56, wherein the subject is a human patient.

58. The method of any one of claims 1-57, wherein the administration is oral or via injection.

59. The method of any one of claims 1-58, wherein the administration is systemic.

60. The method of any one of claims 1-58, wherein the administration is local.

61. The method of claim 60, wherein the administration is rectal.

62. The method of claim 60 or 61, wherein the agent is administered to intestinal epithelial cells of the subject.

63. The method of any one of claims 1-62, wherein the agent is administered to the subject more than once.

64. The method of claim 2, wherein the small molecule blocks the z-nucleic acid binding site of Z-DNA-binding protein 1 (ZBP1).

65. The method of claim 2, wherein the nucleic acid blocks the z-nucleic acid binding site of Z-DNA-binding protein 1 (ZBP1).


SEQUENCE LISTING (4 sequences)

SEQ ID NO: 1 - DNA, 29 bp, Mus musculus
  source 1..29: mol_type = genomic DNA
agtattcaatacctatcccaaagggttcg

SEQ ID NO: 2 - DNA, 29 bp, Mus musculus
  source 1..29: mol_type = genomic DNA
agttgtgttgtccagtttcctgtccatgc

SEQ ID NO: 3 - DNA, 19 bp, synthetic construct
  source 1..19: mol_type = other DNA
ggaagagatctgaggcctg

SEQ ID NO: 4 - DNA, 20 bp, synthetic construct
  source 1..20: mol_type = other DNA
cacttcaggctctggaacag